SEQUENCE LISTING (10 sequences)

SEQ ID NO: 1 - DNA, 23 bp, Homo sapiens
  source 1..23: mol_type = other DNA
ctactgccaaccacggatgaatg

SEQ ID NO: 2 - DNA, 22 bp, Homo sapiens
  source 1..22: mol_type = other DNA
caagggctgttgccatatttcc

SEQ ID NO: 3 - DNA, 23 bp, Homo sapiens
  source 1..23: mol_type = other DNA
tcgaaaaataattgtggggtggc

SEQ ID NO: 4 - DNA, 23 bp, Homo sapiens
  source 1..23: mol_type = other DNA
gctgagttaggaaatttctgggc

SEQ ID NO: 5 - DNA, 22 bp, Homo sapiens
  source 1..22: mol_type = other DNA
aaaaataattgtggggtggcgg

SEQ ID NO: 6 - DNA, 23 bp, Homo sapiens
  source 1..23: mol_type = other DNA
cattccttcctcctgcttattgc

SEQ ID NO: 7 - DNA, 23 bp, Homo sapiens
  source 1..23: mol_type = other DNA
cgtccaaacattagtgtgagtgc

SEQ ID NO: 8 - DNA, 23 bp, Homo sapiens
  source 1..23: mol_type = other DNA
cccttgctccatcatcttacctt

SEQ ID NO: 9 - DNA, 23 bp, Homo sapiens
  source 1..23: mol_type = other DNA
aaggtaagatgatggagcaaggg

SEQ ID NO: 10 - DNA, 23 bp, Homo sapiens
  source 1..23: mol_type = other DNA
ctatatttcccagaagctggggc

(19) United States
(12) Patent Application Publication
(10) Pub. No.: US 2025/0339549 A1
(43) Pub. Date: Nov. 6, 2025

(54) METHODS FOR THE DETECTION AND TREATMENT OF NON-SMALL-CELL LUNG CANCER

(71) Applicant: Board of Regents, The University of Texas System, Austin, TX (US)

(72) Inventors: Samir HANASH, Houston, TX (US); Hiroyuki KATAYAMA, Houston, TX (US); Hanwen XU, Houston, TX (US)

(21) Appl. No.: 19/197,238

(22) Filed: May 2, 2025

Related U.S. Application Data
(63) Continuation of application No. PCT/US2023/078690, filed on Nov. 3, 2023.
(60) Provisional application No. 63/382,234, filed on Nov. 3, 2022.

(51) Int. Cl. A61K 47/68; C07K 16/30; C07K 16/40; G01N 33/573; G01N 33/574

(52) U.S. Cl. CPC A61K 47/68031; A61K 47/6803; A61K 47/6849; C07K 16/30; C07K 16/40; C12Y 301/03001; G01N 33/573; G01N 33/57492; G01N 2333/916

(57) ABSTRACT

Provided are methods of treating a cancer that co-expresses one or more placental alkaline phosphatase (ALPP) proteins and a cancer target antigen in a patient in need thereof, comprising administering to the patient a treatment regimen wherein the treatment regimen comprises administration of one or more standard-of-care inhibitor or anti-proliferative agents that increase cell surface expression of the one or more ALPP proteins and one or more ALPP protein-targeting agents. Also provided are methods of selecting a patient having a cancer that co-expresses one or more placental alkaline phosphatase (ALPP) proteins and a cancer target antigen, comprising: assaying for baseline cell surface expression levels of the one or more ALPP proteins in a biological sample obtained from the patient; and assaying for co-expression of one or more cancer cell surface oncogenic drivers.

### CROSS REFERENCE TO RELATED APPLICATIONS

This application is a bypass continuation of International Application No. PCT/US2023/078690, filed Nov. 3, 2023, which claims the benefit of priority of U.S. Provisional Application No. 63/382,234, filed Nov. 3, 2022, the entirety of which is incorporated herein by reference.

### INCORPORATION OF SEQUENCE LISTING

The sequence listing that is contained in the file named “MDA0076-201BC1-US,” which is 9,844 bytes as measured in Microsoft Windows operating system and was created on May 1, 2025, is filed electronically herewith and incorporated herein by reference.

Alkaline phosphatase placental type (ALPP) and ALPPL2 are closely related and regulated GPI anchored proteins that are expressed on the cell surface in some cancers, while normal tissue expression of ALPP and ALPP2 is largely limited to the placenta. ALPP and ALPPL2 are currently being explored as a cancer therapy target, including immunotherapy trials investigating CAR-T cells targeting ALPP in ovarian cancer (Phase I) and endometrial cancer (Phase 2) in China. In addition, the preclinical efficacy of antibody-drug conjugates targeting ALPP/ALPPL2 has been demonstrated in non-human primates and will serve as the basis for a first-in-human Phase I clinical study.

While antibody-drug conjugates represent a potentially effective treatment option for certain types of cancers, this technology can be limited for cancer types lacking high expression of ALPP/ALPP2. There is a need in the art for methods that more effectively treat cancers expressing ALPP/ALPP2.

### SUMMARY

Provided herein is evidence of enhanced surface expression of ALPP/ALPPL2 in ALPP-expressing cancer cells following treatment with therapeutics that target an oncogenic driver or that inhibit cell proliferation. Enhanced expression increases the susceptibility of cancer cells to therapies directed against ALPP/ALPPL2, resulting in improved anti-cancer efficacy.

Accordingly, provided is a method of treating a cancer that co-expresses one or more placental alkaline phosphatase (ALPP) proteins, e.g., ALPP and/or ALPP2, and a cancer target antigen in a patient in need thereof, comprising administering to the patient a treatment regimen wherein the treatment regimen comprises administration of one or more standard-of-care inhibitor or anti-proliferative agents that increase cell surface expression of the one or more ALPP proteins and one or more ALPP protein-targeting agents.

Also provided is a method of treating a cancer co-expressing alkaline phosphatase (ALPP) and/or ALPP2 proteins and harboring an EGFR activating mutation in a patient in need thereof, comprising administering to the patient an antibody-drug conjugate targeting ALPP and/or ALPP2 in a cell in combination with an EGFR inhibitor.

Also provided is a method for treating a cancer co-expressing alkaline phosphatase (ALPP) and/or ALPP2 proteins and harboring an EGFR activating mutation in a patient in need thereof comprising: identifying the EGFR activating mutation in a cell from a biological sample obtained from the patient; detecting and/or quantifying placental alkaline phosphatase (ALPP) and/or ALPP2 cell surface expression in the cell; administering an antibody-drug conjugate therapy targeting ALPP and/or ALPP2 in the cancer cell in combination with an EGFR inhibitor.

Also provided is a method for treating drug-tolerant or drug-resistant cancer cells co-expressing alkaline phosphatase (ALPP) and/or ALPP2 proteins and in a patient in need thereof comprising: identifying an EGFR activating mutation in a cell from a biological sample obtained from the patient; detecting and/or quantifying ALPP and/or ALPP2 cell surface expression in the cell; administering an antibody-drug conjugate therapy targeting ALPP and/or ALPP2 in the cancer cell in combination with an EGFR inhibitor.

Also provided is a method of preventing emergence of resistance of a cancer cell co-expressing alkaline phosphatase (ALPP) and/or ALPP2 proteins and to an EGFR inhibitor comprising administering an antibody-drug conjugate therapy targeting ALPP and/or ALPP2 in the cancer cell in combination with the EGFR inhibitor.

In some embodiments, prior to administration of the treatment regimen, the method further comprises assaying a biological sample obtained from the patient for baseline cell surface expression levels of the one or more ALPP proteins and for co-expression of one or more cancer cell surface oncogenic drivers.

Also provided is a method of selecting a patient having a cancer that co-expresses one or more placental alkaline phosphatase (ALPP) proteins and a cancer target antigen, comprising: assaying for baseline cell surface expression levels of the one or more ALPP proteins in a biological sample obtained from the patient; and assaying for co-expression of one or more cancer cell surface oncogenic drivers.

These and other aspects of the invention will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF THE DRAWINGS

FIG. 1A-FIG. 1C—(FIG. 1A) Shows a tissue microarray, anti-ALPP IHC staining of normal tissues and lung tumor, in which positivity for ALPP staining was limited to normal testis and lung tumor. (FIG. 1B) Shows a large tissue section, anti-ALPP IHC staining of placenta, mammary gland, and TNBC, in which normal placenta and TNBC showed strong positivity for ALPP staining. (FIG. 1C) Shows cell surface co-expression of EGFR and ALPP/ALPPL2.

FIG. 2A-FIG. 2E—(FIG. 2A) Shows surface co-expression of target proteins and ALPP/ALPPL2 on lung adenocarcinoma cells, H1650 (EGFR mut). (FIG. 2B) Shows changes in cell surface ALPP level associated with tyrosine kinase inhibitor (TKI) treatment on H1650 cells. (FIG. 2C) Shows surface co-expression of target proteins and ALPP/ALPPL2 on breast cancer cells, SKBR3 cells, and MCF7 cells. (FIG. 2D) Shows Gefitinib treatment on ALPP-expressing SKBR3 cells and MCF7 cells lacking ALPP expression. (FIG. 2E) Shows anti-HER-2 antibody treatment to ALPP-expressing SKBR3 cells.

FIG. 3A-FIG. 3D—(FIG. 3A) Shows treatment for proteomic analyses of biotinylated surface proteins in EGFR mut H1650 and PC9 Lung adenocarcinoma cells. (FIG. 3B) Shows surface ALPP expression (spectral abundance) in EGFR mut H1650 and PC9 cells following 24- and 48-hour Gefitinib treatment. (FIG. 3C) Shows immunofluorescence for ALPP expression in EGFR mut H1650 cells following Gefitinib treatment. (FIG. 3D) Shows immunoblots for ALPP following 24- and 48-hour treatment of EGFR mut H1650 and KRAS mut H2291 and H1838 cells with either Gefitinib or epidermal growth factor (EGF).

FIG. 4A-FIG. 4E—(FIG. 4A) Shows anti-ALPP antibody internalization in H1650 cells. (FIG. 4B) Shows that combination treatment of Gefitinib and ALPP-ADC showed the best efficacy in H1650 cells. (FIG. 4C) Shows the synergistic effect of Gefitinib and ALPP-ADC in H1650, H1651, H2291, and H1944 cells. (FIG. 4D) Shows the scheme of a lung orthotopic xenograft in vivo study. (FIG. 4E) Shows that the best efficacy was obtained from Gefitinib (50 mg/kg) and ALPP-ADC (5 mg/kg) combination treatment in the in vivo study.

FIG. 5A-FIG. 5D—(FIG. 5A) Shows surface expression (spectral abundance) of ALPP and EGFR in breast cancer cell lines. (FIG. 5B) Shows immunoblots for ALPP, EGFR, and phosphoEGFR in BT20 and HCC1937 TNBC cells following treatment with Gefitinib (1 μM). (FIG. 5C) Shows representative light field images of BT20 and HCC1937 TNBC cells following treatment with either Gefitinib and ALPP-MMAF-ADC alone or in combination. (FIG. 5D) Shows tumor volume curves for TNBC tumor-bearing mice following treatment with Gefitinib (50 mg/kg) and ALPP-MAFF-ADC (5 mg/kg) alone or in combination. *p-value<0.05; ****p-value<0.0001.

FIG. 6A-FIG. 6E—ALPP and ALPG expression in lung cancer. (FIG. 6A) Spectral abundance (MS/MS events) of ALPP and ALPPL2 in LUAD and SCLC cell lines. (FIG. 6B) Gene expression of ALPP and ALPG (ALPPL2) in lung adenocarcinoma (LUAD), lung squamous cell carcinoma (LUSC) and small cell lung cancer (SCLC) cell lines from the Cancer Cell Line Encyclopedia (CCLE). (FIG. 6C) Gene expression levels of ALPP and ALPG in LUAD and LUSC in The Cancer Genome Atlas (TCGA) datasets. (FIG. 6D) IHC staining of ALPP in NSCLC tumor tissues. (FIG. 6E) Immunofluorescence staining of ALPP in HCC827 and PC9 cell lines.

FIG. 7A-FIG. 7J—EGFR TKI upregulates ALPP expression in LUAD cell. (FIG. 7A) ALPP level in TCGA LUAD datasets with WT or driver mutation in EGFR, KRAS, NF1, and BRAF. (FIG. 7B-FIG. 7C) Immunoblots for ALPP, pAKT-S473, total AKT, PERK-T202/Y204, total ERK, pSTAT1-Y701, pSTAT1-S727, total STAT1, and β-actin in LUAD cancer cell H2291 and H1650 following 24- and 48-hour treatment with or without EGF (10 or 20 ng/mL) or gefitinib (0.5 or 1 μM). (FIG. 7D) Immunoblots for ALPP, EGFR, phosphorylated EGFR (pEGFR Tyr1068) and β-Actin. (FIG. 7E) Flow cytometry analysis of ALPP expression. (FIG. 7F) Immunofluorescence staining of ALPP (Green) and nuclei (Red). (FIG. 7G-FIG. 7H) ALPP expression in gefitinib or osimertinib treated LUAD cell lines. Gene expression data were obtained from Gene Expression Omnibus (GSE75602, GSE193258). (FIG. 7I) Immunoblots of ALPP and β-actin in LUAD cell lines HCC827 and H1650 treated with vehicle control or EGFR inhibitor. (FIG. 7J) Immunoblots of ALPP and β-actin in LUAD cell lines H1395, H1944, HCC2279, H2291, H1650, H1838, and H1651 treated with vehicle control or gefitinib (1 μM) for 48 hours.

FIG. 8A-FIG. 8L—EGFR TKI dephosphorylates FoxO3a to induce ALPP expression. (FIG. 8A) GESA analysis of cell cycle with TCGA LUAD datasets. (FIG. 8B) KEGG pathway analysis using TCGA LUAD datasets. (FIG. 8C) Flow cytometry analysis of cell cycle of HCC827 and PC9 cells treated with vehicle control, gefitnib (30 nM), or osimertinib (30 nM). (FIG. 8D) Immunoblots for ALPP and β-actin in HCC827 and PC9 cell treated with PI3K inhibitor NVP-BKM120 (100 nM), MEK inhibitor AZD8330 (100 nM), and ERK inhibitor SCH772984 (100 nM) for 48 hours. (FIG. 8E) Ingenuity Pathway Analysis of upstream regulators of cells treated with vehicle control or osimertinib. RNAseq data was obtained from Gene Expression Omnibus (GSE193258). (FIG. 8F-FIG. 8G) Immunoblots for ALPP and β-actin in HCC827 and PC9 cell treated with FOXO inhibitor AS1842856 with vehicle or gefitinib for 48 hours. (FIG. 8H) Immunoblots for ALPP and FoxO3a in FoxO3a-overexpressing HCC827 and H1650 LUAD cells treated with vehicle or gefitinib (30 nM) for 48 hours. (FIG. 8I) Immunoblots for ALPP, FoxO3a and phosphorylated FoxO3a (Ser294, Ser425) in HCC827 and H1650 cells treated with 30 nM gefitinib for 6 hours. (FIG. 8J) Immunoblots for FoxO3a in the cytosol and nucleus compartment from gefitinib-treated HC827 and H1650 cell lines. (FIG. 8K) Immunoblots for ALPP, EGFR, pEGFR (Tyr1068), FoxO3a, pFoxO3a (Ser294) and β-Actin in HCC827 and PC9 cells treated with gefitinib (30 nM) or osimertinib (30 nM). (FIG. 8L) ChIP-qPCR assay for the promoter region of ALPP gene in HCC827 and H1650 LUAD cells treated with either vehicle or gefitinib (1 μM) for 6 hours.

FIG. 9A-FIG. 9N—EGFR TKI is required to sustain the transient and reversible upregulation of ALPP. (FIG. 9A) Immunoblots for ALPP, EGFR, pEGFR (Tyr1068), and β-Actin in HCC827 and PC9 cell lines treated with or without (DO) gefitinib or osimertinib for 1 (D1), 2 (D2), and 4 (D4) days. (FIG. 9B) Immunofluorescent staining for ALPP in HCC827 and PC9 cell lines treated with gefitinib or osimertinib for 0 (DO), 1 (D1), 2 (D2), and 4 (D4) days. (FIG. 9C) Flow cytometry analysis of surface ALPP expression in HCC827 and PC9 cells treated with gefitnib (30 nM) and osimertinib (30 nM) for 1, 2, and 4 days. (FIG. 9D) Single cell analysis of NSCLC PC9 cell treated with osimertinib for 0, 3, 7, and 14 days. Data was obtained from Gene Expression Omnibus (GSE150949). (FIG. 9E-FIG. 9F) ALPP expression level (FIG. 9E) and percentage of ALPP+ cells (FIG. 9F) in (FIG. 9D). (FIG. 9G) mRNA level of ALPP in HCC827 and PC9 osimertinib-tolerant cells. Data was obtained from Gene Expression Omnibus (GSE193258). (FIG. 9H) Immunoblots for ALPP, EGFR, pEGFR (Tyr1068), and β-Actin in HCC827 and PC9 parental and DTPC. (FIG. 9I) Immunofluorescent staining for ALPP in HCC827 and PC9 parental and DTPC. (FIG. 9J) Flow cytometry analysis of surface ALPP expression in HCC827 and PC9 parental and DTPCs. (FIG. 9K) Immunoblots for ALPP, EGFR, pEGFR (Tyr1068), and β-Actin in HCC827 and PC9 treated with gefitinib or osimertinib for 48 hours, followed by washout of drugs and resting in TKI-free medium for additional 48 hours. (FIG. 9L) Immunoblots for ALPP EGFR, pEGFR (Tyr1068), and β-Actin. HCC827 and PC9 cells were treated and rested as indicated in (FIG. 9K), followed by re-treatment with gefitinib or osimertinib for 48 hours. (FIG. 9M) Immunoblots for ALPP, EGFR, pEGFR (Tyr1068), and β-Actin in HCC827 and PC9 DPTCs treated with gefitinib for 48 hours, followed by drug withdrawal for additional 48 hours. (FIG. 9N) Immunoblots for ALPP, EGFR, pEGFR (Tyr1068), and β-Actin in HCC827 and PC9 DPTCs rested in TKI-free medium for 48 hours after gefitinib treatment and re-treated with gefitinib for 48 hours.

FIG. 10A-FIG. 10H—EGFR TKI induces ALPP expression in the resistant cancer cells. (FIG. 10A) Immunoblots for ALPP, EGFR, pEGFR (Tyr1068), and β-Actin in HCC827 and PC9 parental (P) and gefitinib-resistant cells (GR) treated with vehicle control or gefitinib (100 nM) for 48 hours. (FIG. 10B) Immunofluorescent staining for ALPP in HCC827 and PC9 parental (P) and gefitinib-resistant cells (GR) treated with vehicle control or gefitinib (100 nM) for 48 hours. (FIG. 10C) Flow cytometry analysis of surface ALPP expression in HCC827 and PC9 parental and gefitinib-resistant cells treated with vehicle control or gefitinib (100 nM). (FIG. 10D) ALPP mRNA expression level in parental and erlotinib-resistant LUAD cell lines HCC827 and HCC4006. Gene expression data were obtained from Gene Expression Omnibus (GSE121634). (FIG. 10E) ALPP mRNA expression level in parental and gefitinib-resistant LUAD cell line PC9. Gene expression data were obtained from Gene Expression Omnibus (GSE75602). (FIG. 10F) ALPP mRNA expression level in PC9 xenograft tumors. Gene expression data were obtained from Gene Expression Omnibus (GSE161584). (FIG. 10G) ALPP expression in EGFR TKI-naïve and -resistant patients. Data were obtained from OncoSG. (FIG. 10H) ALPP expression in NSCLC patients before and post-refractory to osimertinib.

FIG. 11A-FIG. 11E—Combination of EGFR TKI and ALPP-ADC treatment enhances cancer cell-killing in vitro. (FIG. 11A) Cell viability of HCC827 and PC9 treated with gefitinib (50 nM) or osimertinib (50 nM), and αALPP-MMAF (1 μg/ml) or IgG-MMAF (1 μg/ml). (FIG. 11B) Cell viability of HCC827 and PC9 treated with or without gefitinib (50 nM) and osimertinib (50 nM), IgG-MMAF or α-ALPP-MMAF (1 μg/ml). (FIG. 11C) Cell viability of HCC827 and PC9 pretreated with vehicle or gefitinib (50 nM) and osimertinib (50 nM) for 4 days, followed by adding of IgG-MMAF or α-ALPP-MMAF (1 μg/ml). (FIG. 11D) Cell viability (right panel) and crystal violet staining (left panel) of gefitinib-resistant cells treated with IgG-MMAF or α-ALPP-MMAF (5 μg/ml). (FIG. 11E) Cell viability of HCC827 and PC9 cells pretreated with EGFR TKI (100 nM) for 2 days, and then treated with EGFR TKI (100 nM) in combination with IgG-MMAF or α-ALPP-MMAF (5 μg/ml) for 2 days, followed by extended culturing in the presence of EGFR TKI (100 nM) for 20 days.

FIG. 12A-FIG. 12H—Gefitinib treatment potentiates tumoral ALPP expression and enhances anti-cancer efficacy of ALPP-MMAF-ADC in vivo. (FIG. 12A) Confocal imaging of H1650 cells treated with or without Phrodo Red labelled anti-ALPP antibody. (FIG. 12B) Cell viability of H1650 cells treated with serial gefitinib and/or ALPP-MMAF-ADC. (FIG. 12C) Morphology of LUAD cell lines H1650 treated with gefitinib and/or ALPP-MMAF-ADC. (FIG. 12D) H&E staining of tumor tissue and IHC staining of ALPP in tumor tissues from subcutaneous xenograft LUAD mouse model of H1650 cell line treated with or without gefitinib (50 mg/kg). (FIG. 12E) Schematic of treating of orthotopic xenograft LUAD mouse model of H1650 cell line with gefitinib (50 mg/kg) and/or ALPP-MMAF-ADC (5 mg/kg). (FIG. 12F) IVIS imaging of tumors before drug treatment at Day 17 and post-treatment at Day 29. (FIG. 12G) Statistical analysis of tumor burden from (FIG. 12F). *P<0.05, **P<0.01. (FIG. 12H) Hematoxylin and eosin staining of representing tumor sections.

FIG. 13—Proposed Schematic of ALPP surface upregulation in cancer cells by EGFR inhibition.

FIG. 14A-FIG. 14C—ALPP expression in normal tissues. (FIG. 14A) Representative immunohistochemistry (IHC) image for ALPP staining in placental tissues. (FIG. 14B) Immunoblots for ALPP in protein lysates from placenta, lung, breast, and heart tissues. (FIG. 14C) Representative IHC sections for ALPP in various normal tissues.

FIG. 15A-FIG. 15C—EGFR inhibitor upregulate ALPP expression. (FIG. 15A) ALPP level in an east Asian LUAD cohort with WT or driver mutation in EGFR. (FIG. 15B) qPCR for ALPP mRNA levels in HCC827 and PC9 cells treated with vehicle control or gefitinib (30 nM) for 48 hours. (FIG. 15C) ALPP expression in gefitinib or osimertinib treated LUAD cell lines. Gene expression data were obtained from Gene Expression Omnibus (GSE80802, GSE75602, GSE193258).

FIG. 16A-FIG. 16C—FoxO3a is a transcriptional regulator of ALPP. (FIG. 16A) mRNA levels of FOXO1 and FOXO3A in HCC827 and H650 cell lines. Data were obtained from CCLE. (FIG. 16B) mRNA levels of FOXO3A in LUAD cells treated with or without gefitinib. (FIG. 16C) IPA upstream analysis of osimertinib-treated LUAD cell lines. RNAseq data was obtained from Gene Expression Omnibus (GSE193258).

FIG. 17A-FIG. 17B—FoxO3a is a transcriptional regulator of ALPP. (FIG. 17A) potential FoxO3a binding sites (yellow) in the ALPP promoter region (2 kb upstream of ALPP gene). (FIG. 17B) ChIP-qPCR assay for the promoter region of ALPP gene in HCC827 and H1650 LUAD cells treated with either vehicle or gefitinib (1 μM) for 6 hours.

FIG. 18A-FIG. 18D—EGFR TKI is required to sustain the transient and reversible upregulation of ALPP. (FIG. 18A) mRNA level of ALPP in HCC2935 and H1975 osimertinib-tolerant cells. Data was obtained from Gene Expression Omnibus (GSE193258). (FIG. 18B) IC50 of gefitinib and osimertinib in HCC827 and PC9 cells. (FIG. 18C) IC₅₀ of gefitinib and osimertinib in gefitinib- and osimertinib-tolerant cell, respectively. (FIG. 18D) IC₅₀ of gefitinib in gefitinib-resistant HCC827 and PC9 cells.

FIG. 19A-FIG. 19B—EGFR TKI plus ALPP-ADC modality enhances cancer cell-killing efficacy in vitro. (FIG. 19A) Bright field pictures and crystal violet staining of cells from FIG. 11A. (FIG. 19B) Pictures of bright filed and crystal violet staining of HCC827 and PC9 cells treated with EGFR inhibitors (100 nM) in combination with IgG-MMAF or α-ALPP-MMAF (5 μg/ml) for 2 days, followed by extended culturing in the presence of EGFR TKI (100 nM) for 18 days.

FIG. 20—IHC analyses for ALPP in normal tissues of tumor bearing mice following gefitinib treatment.

### DETAILED DESCRIPTION

Provided are methods of treating a cancer that co-expresses one or more placental alkaline phosphatase (ALPP) proteins and a cancer target antigen in a patient in need thereof, comprising administering to the patient a treatment regimen wherein the treatment regimen comprises administration of one or more standard-of-care inhibitor or anti-proliferative agents that increase cell surface expression of the one or more ALPP proteins and one or more ALPP protein-targeting agents. Also provided are methods of selecting a patient having a cancer that co-expresses one or more placental alkaline phosphatase (ALPP) proteins and a cancer target antigen, comprising: assaying for baseline cell surface expression levels of the one or more

ALPP proteins in a biological sample obtained from the patient; and assaying for co-expression of one or more cancer cell surface oncogenic drivers.

Also provided is a method of treating cancer harboring an EGFR activating mutation in a patient in need thereof, comprising administering to the patient an antibody-drug conjugate targeting ALPP and/or ALPP2 in a cell in combination with an EGFR inhibitor. Also provided is a method for treating cancer harboring an EGFR activating mutation in a patient in need thereof comprising: identifying the EGFR activating mutation in a cell from a biological sample obtained from the patient; detecting and/or quantifying placental alkaline phosphatase (ALPP) and/or ALPP2 cell surface expression in the cell; administering an antibody-drug conjugate therapy targeting ALPP and/or ALPP2 in the cancer cell in combination with an EGFR inhibitor. Also provided is a method for treating drug-tolerant or drug-resistant cancer cells in a patient in need thereof comprising: identifying an EGFR activating mutation in a cell from a biological sample obtained from the patient; detecting and/or quantifying ALPP and/or ALPP2 cell surface expression in the cell; administering an antibody-drug conjugate therapy targeting ALPP and/or ALPP2 in the cancer cell in combination with an EGFR inhibitor. Also provided is a method of preventing emergence of resistance of a cancer cell to an EGFR inhibitor comprising administering an antibody-drug conjugate therapy targeting ALPP and/or ALPP2 in the cancer cell in combination with the EGFR inhibitor. Also provided is a method for treating cancer harboring an EGFR activating mutation in a patient in need thereof comprising: administering an antibody-drug conjugate targeting ALPP and/or ALPP2 on the surface of the cancer cells; wherein the cancer cells exhibit increased expression of ALPP and/or ALPP2 relative to a healthy lung cell; wherein the antibody-drug conjugate targeting ALPP and/or ALPP2 comprises an antibody targeting ALPP and/or ALPP2 conjugated to MMAF; wherein the cancer cells comprise an activating mutation in the EGFR gene resulting in resistance to EGFR tyrosine kinase inhibitors.

In some embodiments, the cells comprise increased cell surface expression of ALPP and/or ALPP2 relative to healthy cells.

In some embodiments, the EGFR mutation comprises an exon 19 deletion, a T790M point mutation, and/or an L858R point mutation.

In some embodiments, the cancer having an EGFR mutation is resistant to an inhibitor targeting EGFR.

In some embodiments, the cancer cell is a drug-tolerant persister cell (DTPC) or a drug-resistant cell (DRC).

In some embodiments, the administration of the antibody-drug conjugate therapy targeting ALPP and/or ALPP2 in the cancer cell, and the EGFR inhibitor prevents the drug-tolerant persister cell (DTPC) from developing into a drug-resistant cell (DRC).

In some embodiments, the method treats drug-tolerant persister cells (DTPCs) and drug-resistant cells (DRCs) to prevent the emergence of resistance to EGFR inhibitors.

In some embodiments, the cancer co-expressing alkaline phosphatase (ALPP) and/or ALPP2 proteins is ovarian cancer, breast cancer, cervical cancer, endometrial cancer, pancreatic cancer, gastric cancer, colorectal cancer, lung cancer, urothelial cancer, brain cancer, testicular cancer, seminoma, and mesothelioma.

In some embodiments, the cancer co-expressing alkaline phosphatase (ALPP) and/or ALPP2 proteins is testicular germ cell tumors, uterine corpus endometrial carcinoma, pancreatic adenocarcinoma, pancreatic ductal adenocarcinoma, bladder urothelial carcinoma, triple-negative breast cancer, stomach adenocarcinoma, esophageal carcinoma, uterine carcinosarcoma, rectum adenocarcinoma, head and neck squamous cell carcinoma, lung adenocarcinoma, lung aquamous cell carcinoma, small cell lung cancer, non-small cell lung cancer, clone adenocarcinoma, mesothelioma, and acute myeloid leukemia.

In some embodiments, the lung cancer is non-small cell lung cancer.

In some embodiments, detecting and/or quantifying placental alkaline phosphatase (ALPP) and/or ALPP2 cell surface expression in the cancer cell comprises histological analysis, immunohistochemical (IHC) staining for ALPP protein, a blood-based test, a tissue-based test, or imaging techniques.

In some embodiments, the tissue-based test comprises a tissue biopsy, flow cytometry, immunohistochemistry (IHC), western blot (WB), polymerase chain reaction (PCR), or immunofluorescence (IF).

In some embodiments, the tissue-based test comprises a Mammaprint+Blueprint® test or an Oncotype DX® test.

In some embodiments, the blood-based test comprises Galleri®, circulating tumor cell (CTC) test, a complete blood count (CBC), or a test or assay for measuring circulating proteins, autoantibodies, cell-free circulating DNA, or extracellular vesicle-derived proteins.

In some embodiments, the tissue biopsy is analyzed by hematoxylin and eosin (H&E) staining and/or microscopy.

In some embodiments, the antibody-drug conjugate comprises an antibody targeting ALPP conjugated to a chemotherapeutic drug.

In some embodiments, the EGFR inhibitor comprises a tyrosine kinase inhibitor (TKI).

In some embodiments, the antibody-drug conjugate therapy targeting ALPP and/or ALPP2 is administered in combination with the EGFR inhibitor.

In some embodiments, the EGFR inhibitor is selected from gefitinib, osimertinib, mobocertinib, amivantamab, CLN081, and/or DZD9008.

In some embodiments, the antibody-drug conjugate comprises SGN-ALPV, Adcetris®, Kadcyla®, Besponsa®, Mylotarg®, Polivy®, Padcev®, Enhertu®, Trodelvy®, Blenrep®, Zynlonta™, Akalux®, Aidixi®, and Tivdak®.

In some embodiments, the antibody-drug conjugate therapy targeting ALPP and/or ALPP2 in the cancer cell, and the EGFR inhibitor are administered simultaneously.

In some embodiments, the antibody-drug conjugate therapy targeting ALPP and/or ALPP2 in the cancer cell, and the EGFR inhibitor are administered sequentially.

Alkaline phosphatase, placental type (ALPP) is a membrane-bound glycosylated dimeric enzyme that was first detected in the serum during pregnancy and shown to be originated from the placenta. There are four different isotypes of alkaline phosphatase, placental-type (ALPP), placental-type 2 (ALPP2)), intestinal ALPP (ALPI), and tissue-nonspecific ALPP (ALPL). Of these four isotypes, ALPP and ALPP2 are found to be associated with a large number of human cancers, such as testicular seminoma, ovarian cancer, and endometrial cancer, among others. Other than placental trophoblasts, ALPP/ALPP2 expression on normal tissues is virtually absent, providing an excellent opportunity for developing therapies that require a high degree of tumor specificity. Antibodies directed against ALPP and/or ALPP2 can be conjugated with other classes of drugs such as DNA crosslinking agents or radionuclides (e.g., alpha particles).

A number of characteristics make ALPP and/or ALPP2 an attractive candidate for antigen-targeting immunotherapy: (1) ALPP is a membrane-bound protein and therefore is an accessible cell surface target for specific binding molecules, such as antibodies, (2) Limited expression of ALPP in healthy tissues, but increased expression in malignant tumors suggests that it might serve as a tumor-specific antigen with low off-tumor expression. (3) Alkaline phosphatase activity is reported to induce tumor progression in different cancers, such as prostate cancer, head and neck squamous cell carcinoma, and ovarian cancer. Thus, targeting ALPP as a cancer therapy target may also enhance tumor control by reducing tumor-derived alkaline phosphatase activity.

As described herein, the increased or elevated cell surface expression of ALPP and/or ALPP2 in many cancer types provides a novel opportunity for treatment of these cancers. For those cancers in which the expression of ALPP and/or ALPP2 is elevated, not only can these proteins serve as a cancer therapy target themselves, but additionally, the expression of ALPP and/or ALPP2 can occur in combination with the expression of an activating mutation, e.g., an EGFR-activating mutation, or an oncogenic driver mutation, i.e., a gene implicated in initiating or maintaining cancer. Such a gene may be EGFR or another gene disclosed herein. Increased or enhanced expression of ALPP and/or ALPP2 may also occur in combination with a gene implicated in initiating or maintaining cancer, e.g., an oncogenic driver mutation. Expression of ALPP and/or ALPP2 can be evaluated by detecting and/or quantifying the cell surface expression levels of ALPP and/or ALPP2. Baseline expression of ALPP and/or ALPP2 may provide useful information relating to disease severity and prognosis for treatment of the cancer in the individual. Evaluating baseline cell surface expression levels of ALPP and/or ALPP2 determines whether elevation of ALPP/ALPP2 is present in the cancer, which can serve as a useful first step in determining treatment for the particular cancer. In some embodiments, increased cell surface expression of ALPP and/or ALPP2 increases the susceptibility of cancer cells to therapies directed against ALPP and/or ALPP2. As described herein, the methods of the present disclosure enable prevention of the emergence of resistance of a cancer cell, such as a NSCLC cell, to a drug therapy.

Thus, in some embodiments, a method described herein for treating a cancer expressing elevated levels of ALPP/ALPP2 may initially utilize a step wherein the baseline levels of ALPP and/or ALPP2 are determined before initiating treatment of the cancer. For cancers found to express elevated levels of ALPP and/or ALPP2, a standard-of-care inhibitor treatment or an anti-proliferative agent targeting ALPP and/or ALPP2 may be administered for treatment of the cancer.

In some embodiments, a standard-of-care inhibitor or anti-proliferative agent may be administered to an individual having cancer in order to enhance expression of ALPP and/or ALPP2 on the surface of the cancer cells. In such cases, administration of the standard-of-care inhibitor or anti-proliferative agent serves the purpose of increasing the expression of ALPP and/or ALPP2 on the cell surface in advance of administering an ALPP/ALPP2 protein-targeting agent for treatment of the cancer. This “two-hit” approach increases the expression of the target itself in order to increase the susceptibility of the cancer cells to therapies directed against ALPP and/or ALPP2.

Thus, in some embodiments, measurement of the baseline levels of ALPP and/or ALPP2 protein on the cell surface of the cancer cells may enable determination of an appropriate treatment plan for the cancer. Methods of assaying for baseline cell surface expression levels are well-known in the art, and can include, but are not limited to, histological analysis, immunohistochemical (IHC) staining for ALPP protein, electron microscopy, mass spectrometry analysis, immunofluorescence, a blood-based test, a tissue-based test, or imaging techniques. In some embodiments, detecting and/or quantifying placental alkaline phosphatase (ALPP) and/or ALPP2 cell surface expression, i.e., a baseline value, in the cancer cell comprises histological analysis, immunohistochemical (IHC) staining for ALPP protein, a blood-based test, a tissue-based test, or imaging techniques. Any method capable of determining ALPP and/or ALPP2 levels in a biological sample from an individual can be employed and are intended to be encompassed within the scope of the present disclosure.

A blood-based test may be any blood-based test known or available in the art, such as a Galleri® test, a circulating tumor cell (CTC) test, a complete blood count (CBC), or a test or assay for measuring circulating proteins, autoantibodies, cell-free circulating DNA, or extracellular vesicle-derived proteins. In some embodiments, a blood-based assay or test to detect expression of or levels of ALPP and/or ALPP2 may include the use of a labeled ligand or antibody.

A tissue-based test described herein may be any tissue-based test known or available in the art, such as a tissue biopsy, flow cytometry, immunohistochemistry (IHC), western blot (WB), polymerase chain reaction (PCR), or immunofluorescence (IF). Such tests can utilize specific protein markers or reagents (e.g., staining) and can detect and quantify the amount of ALPP and/or ALPP2 protein present in a biological sample using, e.g., antibodies or any other specific method for determining cell surface expression of ALPP and/or ALPP2. For example, specific tissue-based tests known in the art include, but are not limited to, Mammaprint+Blueprint® test, a Signatara™ test, an Altera Tumor Genomic Profile Test, or an Oncotype DX® test. In some embodiments, the tissue-based test comprises a Mammaprint+Blueprint® test or an Oncotype DX® test.

A biological sample appropriate for the methods described herein can be any biological sample, for example, a blood sample, or a tissue biopsy, or a cell culture sample. Depending on the cancer type, certain biological samples may be more advantageous, e.g., a tissue biopsy for a solid cancer, or a blood-based sample for a hematological cancer, however any biological sample may be useful with the methods described herein.

In some embodiments, the tissue biopsy is analyzed by hematoxylin and eosin (H&E) staining and/or microscopy.

Oncogenic drivers and activating mutations, e.g., an EGFR activating mutation, are well known in the art and can include any gene that is responsible for initiating or maintaining cancer. As described herein, a cell-surface oncogenic driver is a useful cancer target antigen. Targeting of an oncogenic driver as a cancer target antigen may be in combination with targeting of ALPP and/or ALPP2, or may be separate. In some embodiments, ALPP and/or ALPP2 may be targeted for treatment sequentially with an activating mutation or oncogenic driver mutation, e.g., an EGFR-activating mutation, or may be simultaneously targeted in a treatment of the present disclosure, meaning that an antibody-drug conjugate therapy targeting ALPP and/or ALPP2 described herein and an EGFR-activating mutation described herein are administered together or at the same time. In some embodiments, an activating mutation or oncogenic driver mutation, e.g., an EGFR-activating mutation and/or a cell surface oncogenic driver is directly targeted by an inhibitor molecule or by a chemotherapeutic treatment described herein. In some embodiments, an activating mutation, e.g., an EGFR-activating mutation, or an oncogenic driver mutation may be targeted, either alone or in combination with ALPP and/or ALPP2, using any of the cancer therapies described herein. In some embodiments, ALPP and/or ALPP2 is targeted using an antibody-drug conjugate described herein, and the EGFR-activating mutation is targeted using an EGFR inhibitor therapeutic. These therapeutics may be administered simultaneously, or may be administered sequentially. An oncogenic driver useful in treatment of a cancer may be a gene and/or its encoded protein that is implicated in the specific type of cancer, e.g., BRCA1/2 in breast cancer. Some oncogenic driver genes are found mutated in a number of different cancers, while others are specific for one or a few cancer types.

Some common oncogenic drivers that may be useful as described herein, i.e., that may have a mutation causing or contributing to the initiation or maintenance of cancer as described herein, include, but are not limited to, ALK, ARIDIA, BRAF, BRCA1/2, CD22, CD40, CD46, CD74, CDKN2A, DDR1, EFNB2, EGFR, EML4, ENG, EPHA2, EPHB2, EPHB4, ERBB1, ERBB2, ERBB3, FAS, FGFR1, FGFR2, HER2, HRAS, ICAM1, IGF1R, INSR, ITGA4, ITGAV, ITGB1, ITGB3, JAK, KRAS, MAPK, MAP2K1, MEK, MET, MICB, MSTIR, MYC, NF1, NGFR, NRAS, NRP1, NTRK1, PIK3CA, PTEN, PTK7, RAF, RAS, RET, ROR1, ROSI, SEMA4D, STAT, TNFRSF10A, TNFRSF10B, TSC1/2, TP53, UMD, TYRO3, YAP1, and/or YAP2. TP53 is associated with more than 25 different cancer types, while a number of other oncogenic driver genes, e.g., PIK3CA, KRAS, PTEN, ARIDIA, are associated with 15 or more. In some embodiments, one or more of the above oncogenic driver genes may have a mutation causing or contributing to the initiation or maintenance of cancer as described herein.

In some embodiments, the cancer target antigen is a cell surface oncogenic driver.

In some embodiments, the cancer target antigen comprises one or more of CD22, CD40, CD46, CD74, DDR1, EFNB2, ENG, EPHA2, EPHB2, EPHB4, ERBB3, FAS, FGFR2, ICAM1, IGF1R, INSR, ITGA4, ITGAV, ITGB1, ITGB3, MET, MICB, MST1R, NGFR, NRP1, PTK7, RET, ROR1, SEMA4D, TNFRSF10A, TNFRSF10B, TYRO3, YAP1, and/or YAP2.

In some embodiments, the cell surface oncogenic driver is directly targeted by an inhibitor molecule.

The specific oncogenic driver being targeted can be selected by the practitioner or clinician for individualized treatment of a specific cancer type. The present disclosure is not intended to be limited to the list of oncogenic driver genes/proteins described herein, but rather to exemplify the methods of treatment for cancers expressing ALPP and/or ALPP2 described herein.

The most common way to classify cancer is to divide it into 4 categories based on whether it can be removed with surgery and where it has spread: resectable, borderline resectable, locally advanced, or metastatic. Resectable cancer can be surgically removed. The cancerous tumor may be located only in a specific area or organ, or extends beyond it, but it has not grown into important arteries or veins in the area. There is no evidence that the tumor has spread to areas outside of the area. Borderline resectable describes a tumor that may be difficult, or not possible, to remove surgically when it is first diagnosed, but if chemotherapy and/or radiation therapy is able to shrink the tumor first, it may be able to be removed later with negative margins. A negative margin means that no visible cancer cells are left behind. Locally advanced cancer is still located only in the immediate surrounding area around the tumor, but it cannot be surgically removed because it has grown into nearby arteries or veins or to nearby organs. However, there are no signs that it has spread to any distant parts of the body. Metastatic means the cancer has spread beyond the area of the tumor and to other organs, such as the liver or distant areas of the abdomen.

Options for treatment of cancer are established in the literature and can include surgery for partial or complete surgical removal of cancerous tissue, or can involve administering one or more cancer therapies described herein (e.g., chemotherapeutic drugs, immunotherapy drugs, antibody-drug conjugates, therapeutic radiation, etc.).

Chemotherapeutic drugs approved for treatment of cancer are numerous and well known in the art. A number of these are described herein, however it is noted that the present disclosure is not limited to the cancer types or drugs described herein. One of skill in the art would understand that the present disclosure can extend to any appropriate cancer type and any chemotherapeutic drugs or treatments. For example, any cancer type that expresses ALPP and/or ALPP2, or expresses increased cell surface levels of ALPP and/or ALPP2 is within the scope of the present methods.

For example, techniques useful for determination of cell surface expression of ALPP and/or ALPP2, such as detecting and/or quantifying ALPP and/or ALPP2 cell surface expression, can include staining techniques, immunohistochemistry, cell viability assays, microscopy, phosphatase assays, flow cytometry, cell surface biotinylation, proton or chymotrypsin sensitivity assay, antibody labeling assays, ELISA, radioligand binding, cAMP enzyme immunoassays, bioluminescence resonance energy transfer (BRET), CREB phosphorylation assay, live cell staining, imaging, or any others known or available in the art. Staining techniques are known in the art and can involve any stain appropriate for measuring protein, e.g., a fluorescein dye, such as fluorescein-5-isothiocyanate (5-FITC) or fluorescein-5-isothiocyanate (6-FITC), Alexa Fluor 488, Alexa Fluor 647, carboxyfluorescein diacetate (CFSE), R-phycoerythrin (PE), PE-Texas Red, propidium iodide (PI), PE-Cy5, PerCP, PerCP-Cy5.5, PE-Cy7, allophycocyanin (APC), green fluorescent protein (GFP), hematoxylin and eosin (H&E), or any other cell staining techniques or dyes known or available in the art.

In some embodiments, any cancer type that exhibits expression of ALPP and/or ALPP2, or exhibits increased expression of ALPP and/or ALPP2 may be treated according to the methods described herein. In some embodiments, ALPP and/or ALPP2 expression may be increased to a statistically significant degree or level, or may be increased to varying magnitudes, depending on the cancer type. Non-limiting examples of cancer types suitable for treatment as described herein include ovarian, breast, endometrial, pancreatic, gastric, colorectal, lung, urothelial, brain, testicular, seminoma, and mesothelioma. Some specific types of cancers may include, but are not limited to, testicular germ cell tumors, uterine corpus endometrial carcinoma, pancreatic adenocarcinoma, pancreatic ductal adenocarcinoma, bladder urothelial carcinoma, triple-negative breast cancer, stomach adenocarcinoma, esophageal carcinoma, uterine carcinosarcoma, rectum adenocarcinoma, head and neck squamous cell carcinoma, non-small cell lung cancer, clone adenocarcinoma, mesothelioma, and acute myeloid leukemia.

In some embodiments, the cancer that co-expresses one or more placental alkaline phosphatase (ALPP) proteins and a cancer target antigen comprises testicular, ovarian, breast, endometrial, pancreatic, gastric, colorectal, lung, urothelial, brain, testicular, seminoma, or mesothelioma.

In some embodiments, the lung cancer is non-small cell lung cancer.

Provided herein are methods of treating a cancer that expresses ALPP and/or ALPP2, typically at elevated levels. Also provided herein is a method of treating a cancer that co-expresses one or more placental alkaline phosphatase (ALPP) proteins and a cancer target antigen in a patient in need thereof, comprising administering to the patient a treatment regimen, wherein the treatment regimen comprises administration of one or more standard-of-care inhibitor or anti-proliferative agents that increase cell surface expression of the one or more ALPP proteins and one or more ALPP protein-targeting agents. In some embodiments, prior to administration of the treatment regimen, the method further comprises assaying a biological sample obtained from the patient for baseline cell surface expression levels of one or more ALPP proteins and for co-expression of one or more cancer cell surface oncogenic drivers. In some embodiments, the method comprises administration of a treatment regimen, e.g., one or more standard-of-care inhibitor or anti-proliferative agents in order to increase cell surface expression of ALPP and/or ALPP2 in the cancer cells, in order to render the cancer cells more susceptible to one or more ALPP protein-targeting agents.

In some embodiments, the disclosure also provides a method of selecting a patient having a cancer that co-expresses one or more placental alkaline phosphatase (ALPP) proteins and a cancer target antigen, comprising: assaying for baseline cell surface expression levels of the one or more ALPP proteins in a biological sample obtained from the patient; and assaying for co-expression of one or more cancer cell surface oncogenic drivers.

Chemotherapy is widely used as a standard-of-care treatment for cancers and acts to destroy cancer cells and keep them from growing, dividing, and producing more cancer cells. Some chemotherapeutic drugs act by damaging DNA and preventing cell replication, resulting in the death of the cancer cells. As cancer cells typically grow and divide faster than normal, non-cancerous cells, these chemotherapeutic drugs have more of an effect on those actively dividing cancer cells.

As described herein, a treatment regimen that may be used to increase cell-surface expression of ALPP and/or ALPP2 may be any standard-of-care inhibitor or anti-proliferative agent, and includes any chemotherapeutic drug known in the art for treatment of cancer, such as including, but not limited to, Evista (Raloxifene Hydrochloride), Bleomycin, Ifosfamide (Ifex®), Raloxifene Hydrochloride, Soltamox (Tamoxifen Citrate), Tamoxifen Citrate, Abemaciclib, Albumin bound Paclitaxel (Abraxane), (Paclitaxel Albumin-stabilized Nanoparticle Formulation), Ado-Trastuzumab Emtansine, Afinitor (Everolimus), Afinitor Disperz (Everolimus), Alkeran (Melphan), Alpelisib, Altretamine (Hexalen®), Anastrozole, Aredia (Pamidronate Disodium), Arimidex (Anastrozole), Aromasin (Exemestane), Atezolizumab, Avastin (Bevacizumab), Bevacizumab (Avastin), Capecitabine (Xeloda®), Carboplatin, Cisplatin, Cyclophosphamide (Cytoxan®), Docetaxel (Taxotere®), Doxorubicin Hydrochloride, Doxil (Doxorubicin Hydrochloride Liposome), Ellence (Epirubicin Hydrochloride), Enhertu® (Fam-Trastuzumab Deruxtecan-nxki), Epirubicin Hydrochloride, Eribulin Mesylate, Etoposide (VP-16, Vepesid), Everolimus, Exemestane, 5-FU (Fluorouracil Injection), Fam-Trastuzumab Deruxtecan-nxki, Fareston (Toremifene), Faslodex (Fulvestrant), Femara, (Letrozole), Fluorouracil Injection, Fulvestrant, Gemcitabine Hydrochloride, Gemzar® (Gemcitabine Hydrochloride), Goserelin Acetate, Halaven (Eribulin Mesylate), Herceptin Hylecta (Trastuzumab and Hyaluronidase-oysk), Herceptin (Trastuzumab), Hycamtin (Topotecan Hydrochloride), Ibrance (Palbociclib), Infugem (Gemcitabine Hydrochloride), Irinotecan (CPT-11, Camptosar®), Ixabepilone, Ixempra (Ixabepilone), Kadcyla® (Ado-Trastuzumab Emtansine), Keytruda (Pembrolizumab), Kisqali (Ribociclib), Lapatinib Ditosylate, Letrozole, liposomal Doxorubicin (Doxil®), Lynparza (Olaparib), Margenza (Margetuximab-cmkb), Margetuximab-cmkb, Megestrol Acetate, Melphalan, Methotrexate Sodium, mitomycin, Neratinib Maleate, Nerlynx (Neratinib Maleate), Niraparib Tosylate Monohydrate, Olaparib, oxorubicin (Adriamycin®), Oxaliplatin, Paclitaxel (Taxol®), Paclitaxel Albumin-stabilized Nanoparticle Formulation, Palbociclib, Pamidronate Disodium, Pembrolizumab, Pemetrexed (Alimta®), Perjeta (Pertuzumab), Pertuzumab, Pertuzumab, Rubraca (Rucaparib Camsylate), Trastuzumab, and Hyaluronidase-zzxf, Phesgo (Pertuzumab, Trastuzumab, and Hyaluronidase-zzxf), Piqray (Alpelisib), Ribociclib, Sacituzumab Govitecan-hziy, Soltamox (Tamoxifen Citrate), Talazoparib Tosylate, Talzenna (Talazoparib Tosylate), Tamoxifen Citrate, Taxol, Taxotere (Docetaxel), Tecentriq (Atezolizumab), Tepadina (Thiotepa), Thiotepa, Topotecan Hydrochloride, Toremifene, Trastuzumab (Herceptin®), Trastuzumab and Hyaluronidase-oysk, Trexall (Methotrexate Sodium), Trodelvy® (Sacituzumab Govitecan-hziy), Tucatinib, Tukysa (Tucatinib), Tykerb (Lapatinib Ditosylate), Venclexta (venetoclax), Verzenio (Abemaciclib), Vinblastine Sulfate, Xeloda (Capecitabine), Vinorelbine (Navelbine®), Zejula (Niraparib Tosylate Monohydrate), Zoladex (Goserelin Acetate). As would be understood by one of skill in the art, certain cancers benefit from certain chemotherapeutic drugs, or combinations thereof, and therefore the individual treatment plan for a specific cancer may be altered as deemed appropriate by a clinician or practitioner.

In some embodiments, one or more standard-of-care inhibitors or anti-proliferative agents that increase cell surface expression of ALPP and/or ALPP2 are combined together in a combination treatment. Combination treatments may include any combination of treatments for cancer as deemed appropriate by a clinician or physician. For example, cancer treatment options useful for the present methods include one or more of chemotherapeutic drugs, radiation therapies, immunotherapy drugs, DNA cross-linking agents, antibody-drug conjugates, hormone therapies, targeted drug therapies, radionuclides, cryoablation therapies, surgical procedures, or the like.

In some embodiments, the one or more standard-of-care inhibitor or anti-proliferative agents are chosen from Trastuzumab (Herceptin®), Cisplatin, Etoposide (VP-16), Bleomycin, Ifosfamide (Ifex®), Paclitaxel (Taxol®), Carboplatin, Vinblastine, oxorubicin (Adriamycin®), liposomal Doxorubicin (Doxil®), Docetaxel (Taxotere®), Albumin bound Paclitaxel (nab-Paclitaxel, Abraxane®), Altretamine (Hexalen®), Capecitabine (Xeloda®), Cyclophosphamide (Cytoxan®), Gemcitabine (Gemzar®), Irinotecan (CPT-11, Camptosar®), Melphalan, Pemetrexed (Alimta®), Topotecan, Vinorelbine (Navelbine®), 5-fluorouracil (5-FU), irinotecan (Camptosar), Bevacizumab (Avastin), mitomycin, Epirubicin, and Oxaliplatin, and combinations thereof.

Combination treatments known in the art include, but are not limited to, one or more standard-of-care inhibitor or anti-proliferative agents comprises a combination of Bleomycin, Etoposide, and Cisplatin; or a combination of Etoposide and Cisplatin; a combination of VP-16 (Etoposide) or Vinblastine plus Ifosfamide and Cisplatin; a combination of Carboplatin and Paclitaxel; a combination of Cisplatin and Doxorubicin; a combination of Carboplatin and Docetaxel; a combination of Cisplatin and Paclitaxel and Doxorubicin; a combination of Ifosfamide (Ifex®) and Cisplatin; a combination of Ifosfamide (Ifex®) and Paclitaxel; a combination of Trifluridine and Tipiracil (Lonsurf); a combination of Oxaliplatin and 5-FU/leucovorin (FOLFOX); a combination of Oxaliplatin and capecitabine (CAPOX); a combination of 5-FU/leucovorin, Oxaliplatin, and Docetaxel; a combination of Docetaxel or Paclitaxel and either 5-FU or capecitabine; a combination of Cisplatin and either 5-FU or capecitabine; or a combination of Paclitaxel and Carboplatin.

In some embodiments, an antibody-drug conjugate may be used to treat a cancer that overexpresses ALPP and/or ALPP2 as described herein. In some embodiments, the antibody-drug conjugate specifically targets ALPP and/or ALPP2. Antibody-drug conjugates consist of three main components: an antibody drug specific for a particular cancer protein, a cytotoxic chemotherapy drug, and a linker protein connecting the two. In some embodiments, an antibody-drug conjugate useful in accordance with the present disclosure comprises an antibody targeting ALPP and/or ALPP2 conjugated to a chemotherapeutic drug. Any chemotherapeutic drug disclosed herein may be useful for use in an antibody-drug conjugate. Typically administered intravenously, the antibody portion targets the specific cancer protein and is taken up by the cancer cell, where the cytotoxic cancer drug is released and kills the cancer cell. A number of antibody-drug conjugates are known in the art. One example is SGN-ALPV, a novel investigational vedotin antibody-drug conjugate composed of a humanized anti-ALPP/ALPPL2 monoclonal antibody, a protease-cleavable linker, and the microtubule disrupting agent monomethylauristatin E (MMAE). SGN-ALPV is undergoing phase I clinical trials in patients with solid tumors, such as ovarian neoplasms, endometrial neoplasms, non-small cell lung carcinoma, uterine cervical neoplasms, and testicular neoplasms. Other useful antibody-drug conjugates include Tivdak®, which is used to treat cervical cancer, Brentuximab vedotin (Adcetris®)), used for treatment of relapsed Hodgkin and systemic anaplastic large cell lymphomas, both of which exhibit high expression of CD30, Gemtuzumab ozogamicin (Mylotarg®), which targets the CD33 receptor found on certain types of myeloid cells and is approved for relapsed acute myeloid leukemia, Inotuzumab ozogamicin (Besponsa®), which targets the CD22 receptor and is approved for relapsed B-cell precursor acute lymphoblastic leukemia, Polatuzumab vedotin-piiq (Polivy®), which targets the CD79b receptor and is approved in combination with certain chemotherapy regimens for relapsed diffuse large B-cell lymphoma, and Ado-Trastuzumab emtansine (Kadcyla®), which targets the ERBB2 protein on the surface of certain breast cancer cells and is approved to treat advanced breast cancer that expresses this protein. Other antibody-drug conjugates include, but are not limited to, Mylotarg®, Adcetris®, Kadcyla®, Besponsa®, Lumoxiti®, Polivy®, Padcev®, Enhertu®, Trodelvy®, Blenrep®, Zynlonta™, Akalux®, Aidixi®), and Tivdak®.

In some embodiments, the antibody-drug conjugate comprises SGN-ALPV, Adcetris®, Kadcyla®, Besponsa®, Mylotarg®, Polivy®, Padcev®, Enhertu®, Trodelvy®, Blenrep®, Zynlonta™, Akalux®, Aidixi®, and Tivdak®. One of skill in the art would understand that any appropriate antibody-drug conjugate would be useful in accordance with the present disclosure and thus, other antibody-drug conjugates known or available in the art may be useful as described herein.

As described herein, the methods of the present disclosure are useful to treat non-small-cell lung cancer (NSCLC) in a patient wherein the cancer cells exhibit an EGFR-activating mutation. Treatment of NSCLC is known in the art and numerous drug regimens are available. In the present disclosure, administration of an antibody-drug conjugate targeting ALPP and/or ALPP2 may be used in combination with an EGFR inhibitor to treat NSCLC. As described herein in the Examples, such combination treatment may result in an increased or enhanced, i.e., favorable, response of the cancer cells to the treatment when compared to administration of either the antibody-drug conjugate or the EGFR inhibitor alone.

Identification of an EGFR-activating mutation can be accomplished via any techniques known and available in the art, and can include, but are not limited to, DNA sequencing, proteomics analysis, staining techniques, immunohistochemistry, or the like.

Epidermal growth factor receptor (EGFR) tyrosine kinase inhibitor (TKI) therapy is considered the standard-of-care for lung cancers harboring EGFR activating mutations, but with limited performance. EGFR inhibitors are known and available in the art and can include, but are not limited to, gefitinib, osimertinib, mobocertinib, amivantamab, CLN081, and/or DZD9008. Other EGFR inhibitors can be used as deemed appropriate by a clinician or physician. In some embodiments, an EGFR inhibitor may be a tyrosine kinase inhibitor (TKI).

In some embodiments, certain drugs described herein and known in the art, or combinations of drugs or compounds, may be used to treat specific types of cancers that overexpress ALPP and/or ALPP2, either alone, or in combination with a cancer target antigen. For example, in some embodiments, testicular cancer may be treated with Cisplatin, Etoposide (VP-16), Bleomycin, Ifosfamide (Ifex®), Paclitaxel (Taxol®), and/or Vinblastine. In some embodiments, combinations of Bleomycin, Etoposide, and Cisplatin may also be used to treat testicular cancer, along with combinations of Etoposide and Cisplatin, or combinations of VP-16 (Etoposide) or Vinblastine and Ifosfamide and Cisplatin.

In some embodiments, endometrial cancers may be treated with Paclitaxel (Taxol®), Carboplatin, Doxorubicin (Adriamycin®) or liposomal Doxorubicin (Docil®), Cisplatin, or Docetaxel (Taxotere®). Common combination therapies for endometrial cancer include, but are not limited to, Carboplatin and Paclitaxel, Cisplatin and Doxorubicin, Carboplatin and Docetaxel, and Cisplatin, Paclitaxel, and Doxorubicin may be useful.

For carcinoma, treatment with Ifosfamide (Ifex®) is often used, either alone or in combination with either Cisplatin or Paclitaxel. For carcinomas that are HER2-positive, Trastuzumab (Herceptin®) may be added.

For ovarian cancer, combinations of a platinum drug, typically Cisplatin or Carboplatin, and a taxane, e.g., Paclitaxel (Taxol®) or Docetaxel (Taxotere®), Other drugs useful for treatment of ovarian cancer include, but are not limited to, Albumin bound Paclitaxel (nab-Paclitaxel, Abraxane®), Altretamine (Hexalen®), Capecitabine (Xeloda®), Cyclophosphamide (Cytoxan®), Etoposide (VP-16), Gemcitabine (Gemzar®), Ifosfamide (Ifex®), Irinotecan (CPT-11, Camptosar®), Liposomal Doxorubicin (Doxil®), Melphalan, Pemetrexed (Alimta®), Topotecan, and Vinorelbine (Navelbine®

Cervical cancer may be treated with combinations of a chemotherapeutic drug and radiation, referred to herein as concurrent chemoradiation. In some embodiments, Cisplatin or Carboplatin may be given weekly during radiation treatment, or Cisplatin may be combined with 5-fluorouracil (5-FU) and given every 3 weeks during radiation. For recurrent cervical cancer, combinations of Cisplatin, Carboplatin, Paclitaxel (Taxol®), Topotexan, Docetaxel (Taxotere), Ifosfamide (Ifex), 5-fluorouracil (5-FU), irinotecan (Camptosar), gemcitabine (Gemzar), and mitomycin may be used. In some embodiments, a targeted drug therapy, such as Bevacizumab (Avastin) may be added in addition to chemotherapy treatment.

For gastric or stomach cancer may be treated with a number of chemotherapeutic drugs, such as 5-FU (fluorouracil), often given along with leucovorin (folinic acid), Capecitabine, Carboplatin, Cisplatin, Docetaxel, Epirubicin, Irinotecan, Oxaliplatin, Paclitaxel, or Trifluridine and Tipiracil (Lonsurf), which is a combination drug in pill form. For earlier-stage cancers, some common drug combinations that may be used before and/or after surgery include, but are not limited to, Oxaliplatin and 5-FU/leucovorin (FOLFOX), Oxaliplatin plus capecitabine (CAPOX), 5-FU/leucovorin, Oxaliplatin, and Docetaxel, Docetaxel or Paclitaxel combined with either 5-FU or capecitabine, Cisplatin in combination with either 5-FU or capecitabine, or Paclitaxel and Carboplatin. For cases when chemo is given with radiation after surgery, a single drug such as 5-FU or capecitabine may be used. In some embodiments, for advanced stomach or gastric cancer, many of the same combinations of drugs can be used, although often combinations of two drugs, rather than three, to try to reduce side effects may be useful. Some of the most commonly used combinations include, but are not limited to, Oxaliplatin and 5-FU/leucovorin (FOLFOX), Oxaliplatin plus capecitabine (CAPOX), Cisplatin combined with either 5-FU or capecitabine, Irinotecan combined with 5-FU/leucovorin (FOLFIRI), Paclitaxel combined with either Cisplatin or Carboplatin, Docetaxel combined with Cisplatin, Epirubicin, either Cisplatin or Oxaliplatin, and either 5-FU or capecitabine, and combinations of Docetaxel, 5-FU, and either Cisplatin, Carboplatin, or Oxaliplatin.

Radionuclide therapy uses radioactive substances called radiopharmaceuticals to treat cancer. Radionuclides are introduced into the body by various means and localize to specific locations, organs, or tissues depending on their properties and administration routes. Radionuclides can be provided in a variety of particle types, including alpha, beta, gamma, or combinations of these.

In some embodiments, a radionuclide is an alpha particle, such as Polonium-210, Bismuth-213, or Uranium-238. In some embodiments, the radionuclide comprises an alpha particle selected from Polonium-210 and Uranium-238.

In some embodiments, a radionuclide is a beta particle, such as Strontium-90, Thallium-201, Carbon-14, and Tritium. In some embodiments, a radionuclide is a gamma particle, such as Barium-133, Cadmium-109, Cobalt-57, Cobalt-60, Europium-152, Manganese-54, Sodium-22, Zinc-65, and Technetium-99m. In some embodiments, a radionuclide may be a combination of these particles, such as Cesium-137 and Americum-241. Any radionuclides known or available in the art may be used in accordance with the methods described herein.

In some embodiments, combinations of radionuclides can be used, wherein each radionuclide in a combination radionuclide emits radiation at a different wavelength such that each individual radionuclide is separately distinguishable.

In some embodiments, a drug treatment described herein may be administered in a clinical setting or may be administered in an alternate setting as deemed appropriate by a clinician or practitioner. In some embodiments, the one or more standard-of-care inhibitor or anti-proliferative agents that increase cell surface expression of the one or more ALPP proteins and the one or more ALPP protein-targeting agents are administered simultaneously or sequentially.

In some embodiments, the one or more ALPP protein-targeting agents comprises an antibody-drug conjugate, an immunotherapy, a DNA cross-linking agent, or a radionuclide, or a combination thereof.

Any of the treatments described herein for treatment of a cancer that expressed ALPP and/or ALPP2, either alone or in combination with a cancer target antigen, e.g., an oncogenic driver described herein, can be administered to a patient in need thereof (e.g., a patient that has cancer) alone or in combination with (i.e., by co-administration or sequential administration) a second drug regimen or other therapeutic treatments or drugs for treating cancer (e.g., chemotherapeutic or immunotherapy drugs or treatments). In one embodiment, the additional therapeutic treatments or drugs are included in a pharmaceutical composition as described herein. In other embodiments, the additional therapeutic treatments or drugs are co-administered, administered concurrently, or administered sequentially in separate or distinct compositions.

As would be understood by one of skill in the art, a treatment described herein is administered in any form necessary or useful to the subject for treatment of cancer, for example, a liquid (e.g., injectable and infusible solutions), a semi-solid, a solid, an aqueous solution, a suspension, an emulsion, a gel, a magma, a mixture, a tincture, a powder, a capsule, a dispersion, a tablet, a pellet, a pill, a powder, a liposome, a lozenge, a troche, a liniment, an ointment, a lotion, a paste, a suppository, a spray, an inhalant, or the like. In some embodiments, a drug as described herein for treatment of cancer may be administered in a liquid or aqueous form for injection into a patient, or in a pill or tablet form for oral administration. The dosage form of a drug described herein can depend on the intended mode of administration and therapeutic application. Typically, dosage forms for the drug treatments described herein are in the form of injectable or infusible solutions, or in the form of a pill for oral administration.

In some embodiments, a drug as described herein for treatment of cancer in a patient may be administered by any route or mode of administration, such as intraperitoneal, intravenous, oral, sublingual, rectal, vaginal, ocular, otic, nasal, cutaneous, enteral, epidural, intra-arterial, intravascular, nasal, respiratory, subcutaneous, topical, transdermal, intramuscular, or the like. In other embodiments, a second drug regimen, such as a chemotherapeutic drug regimen, may be in the form of an aqueous solution for intravenous administration.

Unless otherwise specified herein, the methods described herein can be performed in accordance with the procedures exemplified herein or routinely practiced methods well known in the art. The following sections provide additional guidance for practicing the methods of the present disclosure.

In some embodiments, a cancer therapy to be administered to an individual for treatment of cancer may be provided as a composition. In some embodiments, the methods described herein may involve compositions to be administered as a single composition. In some embodiments, each drug may be administered separately (while still being administered concurrently), i.e., in separate solutions or drug forms as described herein. Pharmaceutical formulation is well established and known in the art.

In some embodiments, a drug or composition for use in the methods described herein may be formulated with excipient materials, such as sodium citrate, sodium dibasic phosphate heptahydrate, sodium monobasic phosphate, Tween-80, and/or a stabilizer. The drug or composition for use in the methods described herein can be provided, for example, in a buffered solution at a suitable concentration and can be stored at an appropriate temperature to maintain the efficacy of the drug(s), for example a temperature of 2-8° C. In some other embodiments, the pH of the composition is between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

A pharmaceutical composition for the described methods can also include agents that reduce aggregation of the drug when formulated. Examples of aggregation reducing agents include one or more amino acids selected from the group consisting of methionine, arginine, lysine, aspartic acid, glycine, and glutamic acid. The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

As described above for a cancer treatment of the present disclosure, compositions for use with the described methods can be administered by a parenteral mode (e.g., intravenous, subcutaneous, intraperitoneal, or intramuscular injection). The phrases “parenteral administration” and “administered parenterally” as used herein mean modes of administration, usually by injection, and include, without limitation, intravenous, intramuscular, intra-arterial, intrathecal, intracapsular, intraocular, intracardiac, intradermal, intraperitoneal, transtracheal, subcutaneous, subcuticular, intraarticular, subcapsular, subarachnoid, intraspinal, epidural and intrasternal injection, and infusion.

A composition for use with the described methods can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, compositions may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In some embodiments, a composition is formulated in sterile distilled water or phosphate buffered saline. The pH of the pharmaceutical formulation may be between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

A cancer treatment for treating a cancer that expressed ALPP and/or ALPP2, either alone or in combination with a cancer target antigen, e.g., an oncogenic driver described herein, can be provided in a kit. In one embodiment, the kit includes (a) a container that contains the individual cancer therapies as described herein, and optionally (b) informational material. The informational material can be descriptive, instructional, marketing, or other material that relates to the methods described herein and/or the use of the agents for therapeutic benefit.

In some embodiments, the kit also includes a second agent (e.g., a chemotherapeutic or immunotherapy drug described herein) for treating cancer described herein. For example, the kit includes a first container that contains a standard-of-care inhibitor or anti-proliferative agent, and a second container that includes a second drug regimen, e.g., a chemotherapeutic or immunotherapy drug or combination of drugs.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the cancer treatment, as well as the chemotherapeutic or immunotherapy drug, e.g., in a suitable dose, dosage form, or mode of administration (e.g., a dose, dosage form, or mode of administration described herein), to treat a subject who has cancer. The information can be provided in a variety of formats, include printed text, computer readable material, video recording, or audio recording, or information that provides a link or address to substantive material, e.g., on the internet.

In addition to the cancer treatments or agents, the kit can include materials or reagents necessary for determining the baseline level of ALPP and/or ALPP2, along with other ingredients, such as a solvent or buffer, a stabilizer, or a preservative. The cancer treatments can be provided in any form described herein, e.g., liquid, dried or lyophilized form, substantially pure and/or sterile. In some embodiments, when the agents are provided in a liquid solution, the liquid solution is an aqueous solution. When the agents are provided as a lyophilized product, the lyophilized powder is generally reconstituted by the addition of a suitable solvent. The solvent, e.g., sterile water or buffer (e.g., PBS), can optionally be provided in the kit.

The kit can include one or more containers for the drugs or compositions. In some embodiments, the kit contains separate containers, dividers, or compartments for the drugs and informational material. For example, the cancer treatments can be contained in a bottle, vial, or syringe, and the informational material can be contained in a plastic sleeve or packet. In other embodiments, the separate elements of the kit are contained within a single, undivided container. For example, the cancer treatments or agents may be contained in a bottle, vial, or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the cancer treatment(s) in a desired ratio. For example, the kit may include a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight.

The kit optionally includes a device suitable for administration of the cancer treatments, e.g., a syringe or other suitable delivery device. The device can be provided pre-loaded with one or both of the agents or can be empty, but suitable for loading.

### Definitions

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Any discrepancy between the disclosure of a reference discussed herein and the present disclosure shall be resolved in favor of the present disclosure. Further, the dates of publication provided may be different from the actual publication dates which may need to be independently confirmed.

Unless defined otherwise, all technical and scientific terms used herein have the meaning commonly understood by one of ordinary skill in the art to which the disclosure pertains. Some specific terminology relevant to the description of the present disclosure is defined below.

As used in this specification and the appended claims, the singular forms “a,” “an,” and “the,” along with similar references used in the context of describing a particular embodiment (especially in the context of certain of the following claims), can be construed to cover both the singular and the plural, unless specifically noted otherwise. Thus, for example, “an active agent” refers not only to a single active agent, but also to a combination of two or more different active agents, “a dosage form” refers to a combination of dosage forms, as well as to a single dosage form, and the like. In some embodiments, the term “or” as used herein, including the claims, is used to mean “and/or” unless explicitly indicated to refer to alternatives only or the alternatives are mutually exclusive.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. In some embodiments, “about” refers to a specified value+/−10%.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

As used herein, “co-administration” refers to the combined administration of one or more drugs with another. In some embodiments, both drugs are administered at the same time. Co-administration may also refer to any particular time period of administration of either drug, or both drugs. For example, as described herein, a drug may be administered hours or days before administration of another drug and still be considered to have been co-administered. In some embodiments, co-administration may refer to any time of administration of either drug such that both drugs are present in the body of a patient at the same. In some embodiments, either drug may be administered before or after the other, so long as they are both present within the patient for a sufficient amount of time that the patient received the intended clinical or pharmacological benefits.

As used herein, “anti-cancer therapy” refers to any therapy for treatment or prevention of cancer. For example, an anti-cancer therapy as described herein may refer to a chemical such as a chemotherapeutic drug or compound, or combination of drugs or compounds for cancer treatment. In some embodiments, an anti-cancer therapy may refer to a drug or compound, or combination of drugs or compounds, that leads to induced immunogenicity of the cell (e.g., a cancer cell or a cancer stem cell). In some embodiments, inducing immunogenicity of a cancer cell can include, for example, any means for inducing the cancer cell(s) to progress to a more differentiated state associated with a more limited capacity to proliferate and a finite lifespan. This can be achieved by, e.g., upregulation or enhancement of expression of self and tumor antigens that make such cells more susceptible to immune-based therapies.

As used herein, a “DNA-damaging agent” refers to an agent or compound that introduces damage to the DNA of a cancer cell, through a number of mechanisms. For example, DNA damage can result from errors in DNA replication or prevention of DNA repair machinery, among others, which can include double-strand break (DSB) repair through the homologous recombination (HR) and non-homologous end joining (NHEJ) pathways. DNA damaging agents are widely used in oncology to treat both hematological and solid cancers. Some commonly used modalities include, without limitation, ionizing radiation, platinum drugs (e.g., Cisplatin, Oxaliplatin, and Carboplatin), cyclophosphamide, chlorambucil, Doxorubicin, and temozolomide. Other commonly used drugs for treatment of cancers are described in detail herein. A “DNA-damaging agent” as used herein may refer to a chemotherapy agent or regimen, or may refer to any agent known or available in the art capable of introducing DNA damage to a cancer cell.

As used herein, “EGFR” refers to the EGFR gene, also known as the “Epidermal Growth Factor” gene and “HER1.” A number of mutations in EGFR are known to be associated with cancer, including NSCLC.

As used herein, an “EGFR-activating mutation” refers to a mutation in the EGFR gene in the DNA of a cell in a patient that results in aberrant expression of the EGFR gene, i.e., a mutation that “activates” expression of the EGFR gene. EGFR-activating mutations are most commonly found in non-small-cell lung cancer (NSCLC), glioblastoma, and colorectal cancer. EGFR-activating mutations are typically found in exons 18-21 of the EGFR gene, which is part of the gene encoding the tyrosine kinase domain of the EGFR protein. EGFR mutations in accordance with the present disclosure may be point mutations, missense mutations, substitution mutations, or the like. In NSCLC, the most common activating mutations observed are exon 19 deletions and an L858R point mutation in exon 21. Other EGFR mutations found in lung cancer include, but are not limited to, C797S mutation, and EGFR exon 20 insertions, a T790M substitution, and L858R missense mutation.

As described herein, the present methods are useful for treating NSCLC in which a patient or subject has an EGFR-activating mutation. For an EGFR-activating mutation, the genetic mutation in question renders the cancer cell resistant to an inhibitor. For example, a non-small-cell lung cancer having an EGFR mutation is resistant to an inhibitor targeting EGFR, e.g., an EGFR inhibitor. Some patients who would benefit from the present methods would be a patient having NSCLC that is resistant to an inhibitor targeting EGFR. Combination therapies comprising administering an antibody-drug conjugate targeting ALPP and/or ALPP2 and an EGFR inhibitor as described herein.

As used herein, a “drug-resistant cell” or “DRC” refers to a cell that has developed resistance to a particular drug. As described herein, a non-small-cell lung cancer cell may be a drug-resistant cell, or may develop into a drug-resistant cell.

As used herein, a “drug-tolerant persister cell” or “DTPC” refers to a cell that exhibits a reversible phenotype. DTPCs can resume proliferation and drug sensitivity after discontinuation of a drug treatment. DTPCs can eventually acquire several types of drug-resistant mechanisms under continuous treatment. As described herein, a non-small-cell lung cancer cell may be a drug-resistant cell, or may develop into a drug-resistant cell. The methods described herein prevent a drug-tolerant persister cell (DTPC) from developing into a drug-resistant cell (DRC). The methods described herein, i.e., administration of an antibody-drug conjugate targeting ALPP and/or ALPP2, in combination with an EGFR inhibitor, also treat drug-tolerant persister cells (DTPCs) and drug-resistant cells (DRCs) by preventing the emergence of resistance to an EGFR inhibitor as described herein.

A pharmaceutical composition(s) comprising one or more cancer treatments described herein may include a “therapeutically effective amount” of the cancer treatments as described herein. The term “therapeutically effective amount,” “pharmacologically effective dose,” “pharmacologically effective amount,” or simply “effective amount” may be used interchangeably and refers to that amount of an agent effective to produce the intended pharmacological, therapeutic or preventive result, e.g., a reduction of cancerous cells or lessened cancer cell burden (i.e., reduction in number of cancer cells), tumor size, tumor density, lymph node involvement, metastases, cancer recurrence or relapse, or associated symptoms in the patient. The pharmacologically effective amount results in the amelioration of one or more symptoms of a disorder (e.g., a hematological cancer), or prevents the advancement of a disorder, or causes the regression of the disorder, or prevents the disorder. Such effective amounts can be determined based on the effect of the administered agent, e.g., cancer treatment described herein, or the combinatorial effect of agents if more than one agent is used, e.g., a cancer treatment described followed by a second cancer treatment or agent described herein. A therapeutically effective amount of an agent may also vary according to factors such as the disease stage, state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects. In some examples, an “effective amount” is one that treats (including prophylaxis) one or more symptoms and/or underlying causes of cancer. In some examples, an effective amount is a therapeutically effective amount. In some examples, an effective amount is an amount that prevents one or more signs or symptoms of a particular disease or condition from developing.

As used herein, “gene expression” or “expression” refers to the process of gene transcription, translation, and post-translational modification.

As used herein, an “immunotherapy drug” refers to a drug or compound that stimulates or suppresses the immune system to help the body fight cancer, infection, and/or other diseases. Immunotherapy drugs can be antibodies, e.g., a monoclonal antibody. In some embodiments, an immunotherapy drug may be an immune checkpoint inhibitor drug, e.g., a drug that targets the programmed cell death protein (PD-1) or its ligand, PDL-1, and/or cytotoxic T lymphocyte antigen 4 (CTLA-4) receptors. For example, useful immunotherapy drugs include, but are not limited to, Pembrolizumab (Keytruda®), Ipilimumab (Yervoy®), Pembrolizumab (Keytruda®), Nivolumab (Opdivo®), Atezolizumab (Tecentriq®), Pidilizumab (CT-011), Toripalimab (JS-001), Avelumab (Bavencio®), Tislelizumab (BGB-A317), Durvalumab (Imfinzi®), and Cemiplimab (Libtayo®). Immunotherapy drugs targeting PD-1 include, but are not limited to, Pembrolizumab (Keytruda®), Nivolumab (Opdivo®), and Cemiplimab (Libtayo®). Immunotherapy drugs targeting PD-L1 include, but are not limited to, Atezolizumab (Tecentriq®), Avelumab (Bavencio®), and Durvalumab (Imfinzi®). Immunotherapy drugs targeting CTLA-4 include, but are not limited to, Ipilimumab (Yervoy®). Additional immunotherapy drugs targeting these checkpoint inhibitors are known and available in the art, and are encompassed within the scope of the present disclosure.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration or comparable foreign regulatory agencies. “Pharmacologically active” (or simply “active”) as in a “pharmacologically active” (or “active”) derivative or analog, refers to a derivative or analog having the same type of pharmacological activity as the parent compound and approximately equivalent in degree. Some pharmacologically active derivatives may have improved pharmacological activity. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt.

As used herein, “reducing” refers to a lowering or lessening, such as reducing cancer cell burden. In some embodiments, administration of a cancer treatment as described herein may result in “reduced” or lessened cancer cell burden (i.e., reduction in number of cancer cells), tumor size, tumor density, blast cell involvement, proliferation, proportion of quiescent (G₀) cells, lymph node involvement, metastases, or associated symptoms in the patient compared to a patient not having been administered such drugs. “Reducing” may also refer to a reduction in disease symptoms as a result of a treatment as described herein, either alone, or co-administered with another drug.

As used herein, the term “cancer” refers to a malignant neoplasm characterized by the abnormal proliferation of cells, the growth of which cells exceeds and is uncoordinated with that of the normal tissues around it.

As used herein, an “oncogenic driver” refers to a gene that is implicated in initiating or maintaining cancer. A mutation that contributes to cancer tends to affect three main types of genes, e.g., proto-oncogenes, tumor suppressor genes, and DNA repair genes. These changes are sometimes called “drivers” of cancer. These mutations are often found in genes that encode signaling proteins that are critical for maintaining normal cellular proliferation and survival.

As used herein, the term “subject” or “patient” refers to a mammal, preferably a human, having cancer, and for whom further treatment can be provided.

As used herein, “healthy” refers to an individual who does not have cancer.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including but not limited to laser fluorimetry.

As used herein, “subject” or “individual” or “patient” refers to any patient for whom or which therapy is desired, and generally refers to the recipient of the therapy. A “subject” or “patient” refers to any animal classified as a mammal, e.g., human and non-human mammals. Examples of non-human animals include dogs, cats, cattle, horses, sheep, pigs, goats, rabbits, etc. Unless otherwise noted, the terms “patient” or “subject” are used herein interchangeably. In some embodiments, a subject amenable for therapeutic applications may be a primate, e.g., human and non-human primates.

The terms “treating” and “treatment” or “alleviating” as used herein refer to reduction or lessening in severity and/or frequency of symptoms, elimination of symptoms and/or underlying cause, and improvement or remediation of damage. In certain aspects, the term “treating” and “treatment” as used herein refer to the prevention of the recurrence of symptoms. In other aspects, the term “treating” and “treatment” as used herein refer to the prevention of the underlying cause of symptoms associated with a disease or condition, such as hematological cancer. The phrase “administering to a patient” refers to the process of introducing a composition or drug into the patient via an art-recognized means of introduction. “Treating” or “alleviating” also includes the administration of compounds or agents to a subject to prevent or delay the onset of the symptoms, complications, or biochemical indicia of a disease (e.g., cancer), alleviating the symptoms or arresting or inhibiting further development of the disease, condition, or disorder. Subjects in need of treatment include those already suffering from the disease or condition, those previously suffering from the disease or condition and at risk of recurrence, as well as those being at risk of developing the disease or condition

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of ordinary skill in the art should, in light of the present disclosure, appreciate that many changes can be made in the specific embodiments which are disclosed and still obtain a like or similar result without departing from the spirit and scope of the disclosure.

To exemplify the presently claimed methods, treatment of EGFR-expressing lung adenocarcinoma cells with small-molecule EGFR inhibitors to amplify ALPP cell-surface expression is demonstrated in vitro and in vivo. In addition, it is demonstrated that treatment with other therapeutic agents enhances ALPP protein expression. Treatment of lung adenocarcinoma-bearing mice with the EGFR inhibitor gefitinib, together with an anti-ALPP antibody conjugated with Monomethylauristatin F (MMAF), resulted in significantly improved anti-cancer efficacy compared to either gefitinib or MMAF treatment alone. Similar combined efficacy of gefitinib plus anti-ALPP-MMAF was observed in vitro and in vivo models of triple-negative breast cancer.

As described herein below, a diagnostic test for combined therapy that includes anti-ALPP/ALPPL2 agents comprises steps of assessing (1) baseline ALPP/ALPPL2 surface expression in cancer cells as measured through histological analysis of tissue biopsies or blood-based tests for tumor disseminated evidence of ALPP/ALPPL2 expression; and (2) co-expression of cancer cell surface oncogenic drivers that are directly targetable with inhibitors.

In addition, a “two-hit” combination therapeutic strategy is demonstrated, comprising (1) treatment with standard-of-care inhibitor anti-proliferative agent(s) to enhance cancer cell surface ALPP/ALPPL2 expression; and (2) anti-ALPP/ALPPL2 antibody-drug conjugates or immunotherapies or radionuclide therapies.

### Example 1

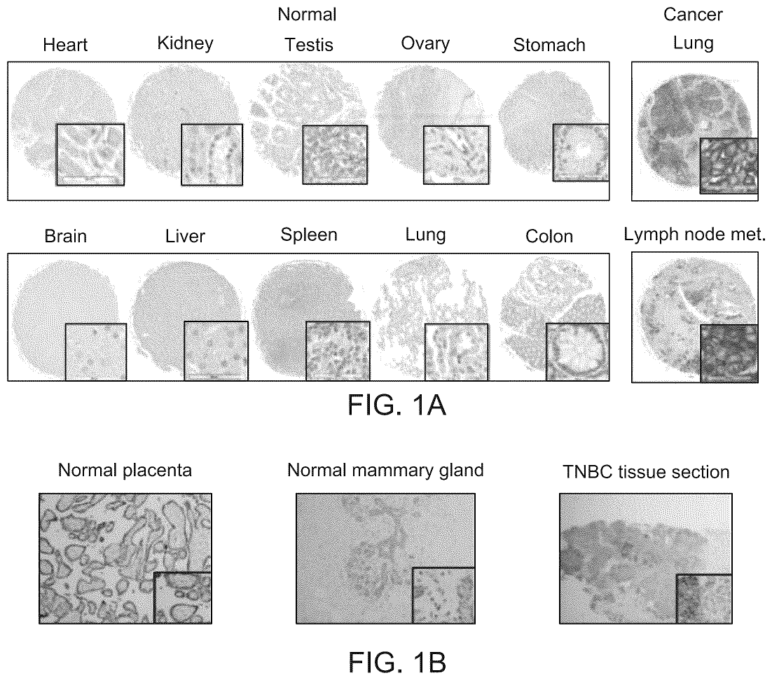

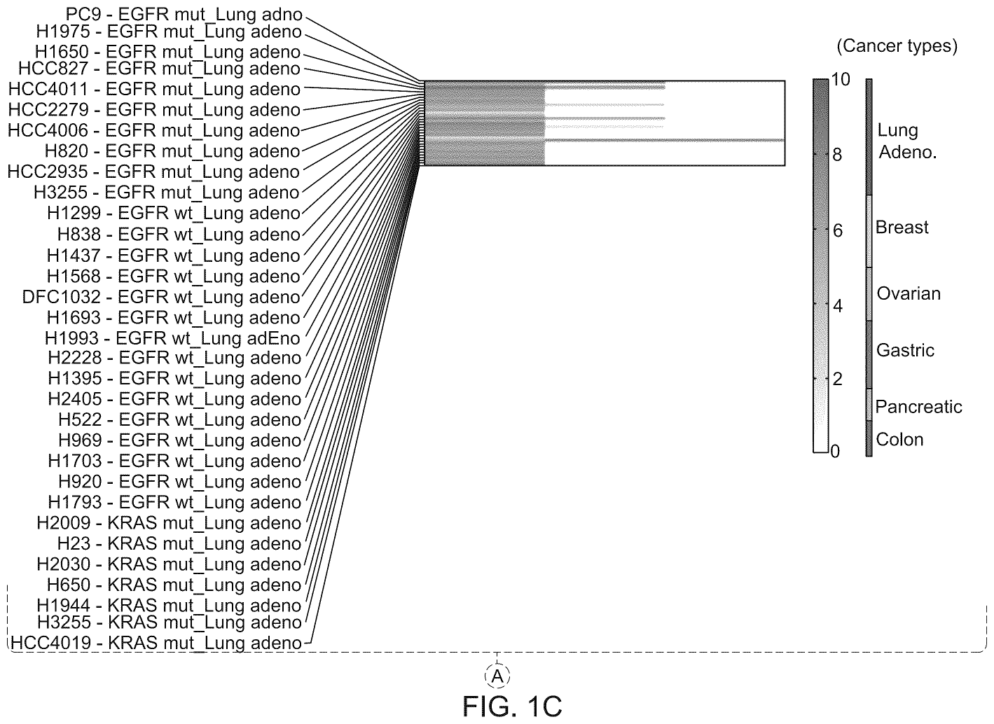

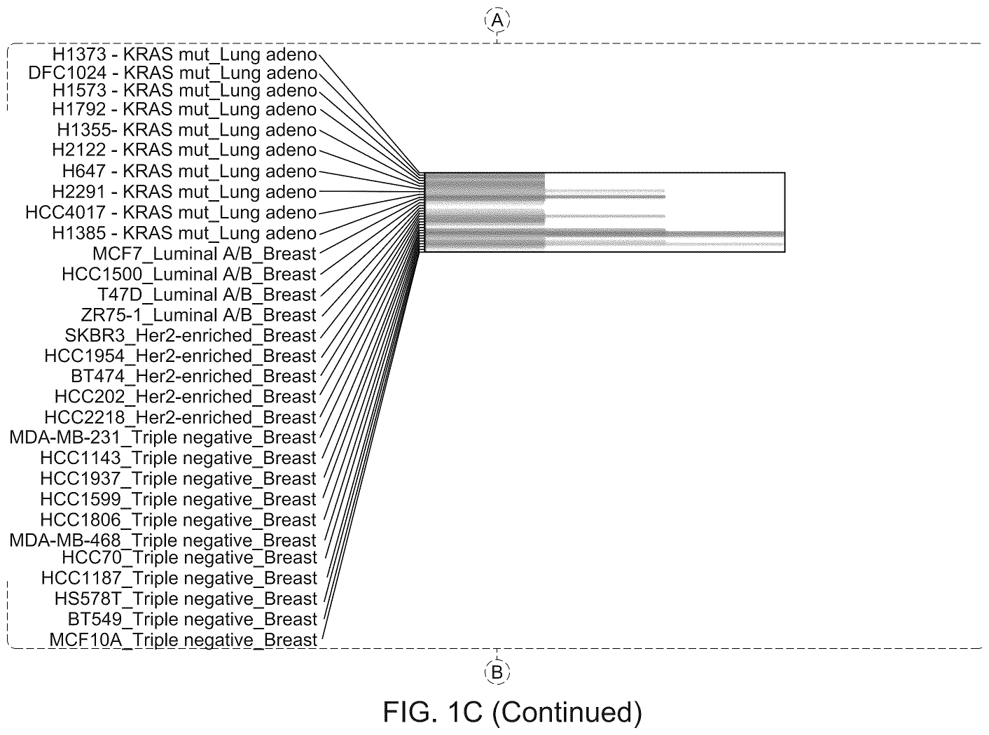

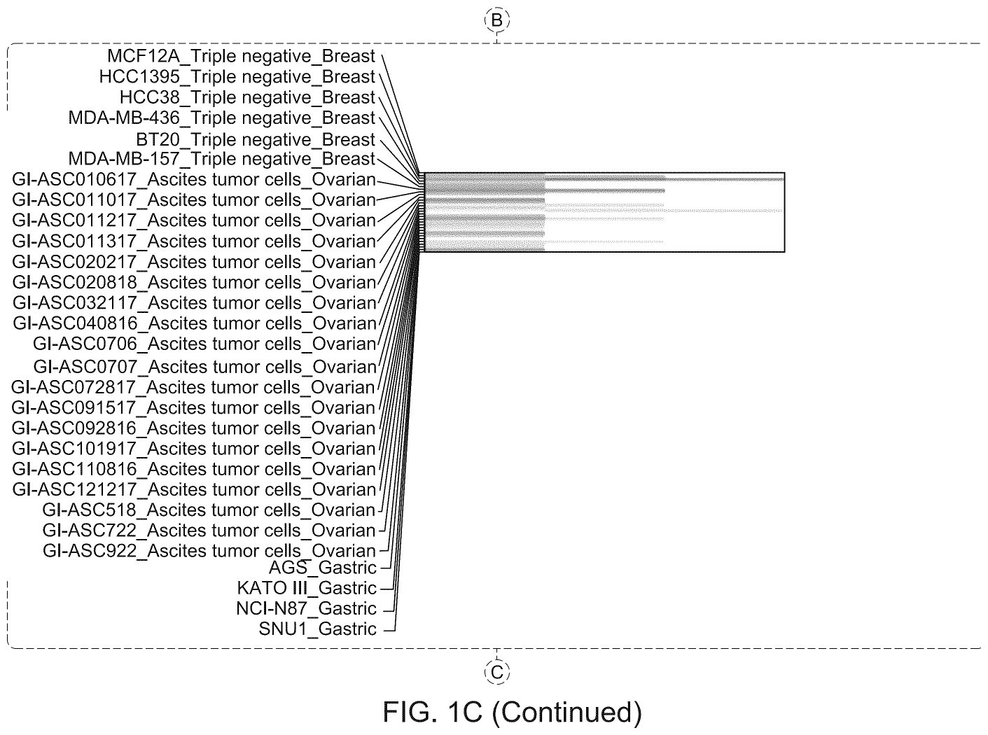

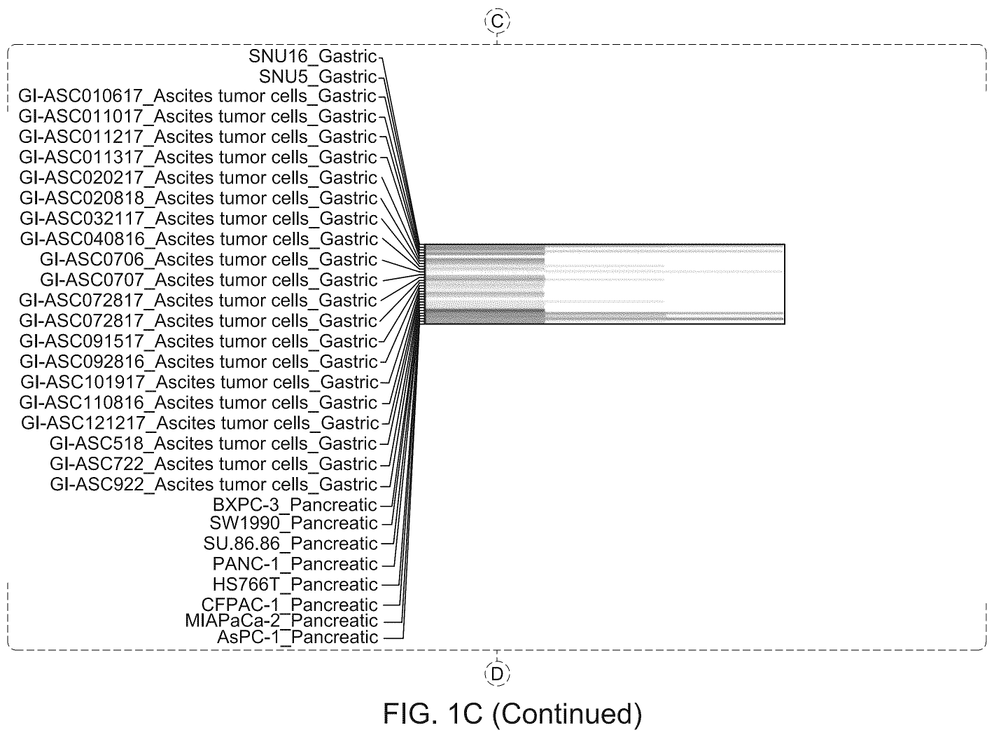

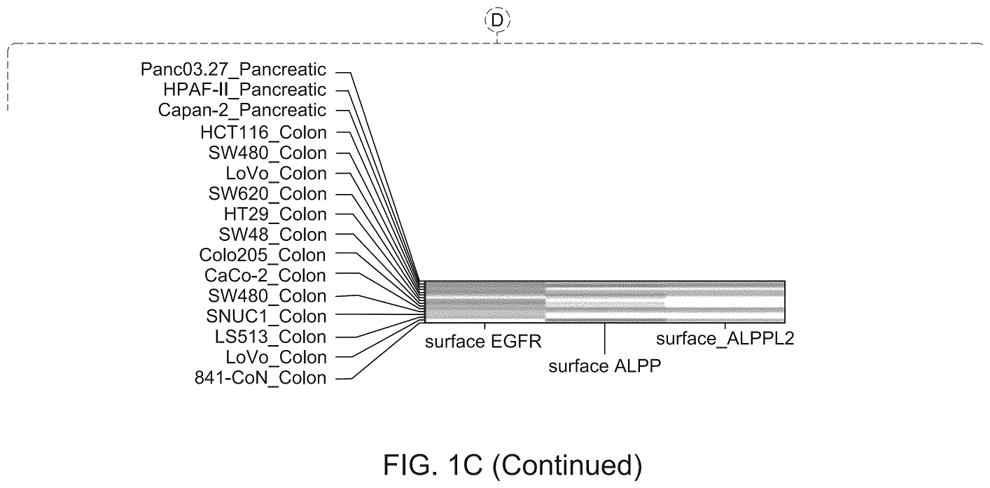

Immunohistochemical (IHC) analyses for ALPP protein in normal tissues indicates limited expression in testis and placenta, while heart, kidney, ovary, stomach, brain, liver, spleen, lung, colon, and mammary gland were negative (FIG. 1A, B). Inversely, representative IHC staining of ALPP in lung and breast tumors indicates strong positivity (FIG. 1A, B). The representative target EGFR and ALPP/ALPPL2 expression via mass spectrometry analysis of biotinylated surface proteins frequently co-expressed in 7 cancer types is shown in FIG. 1C.

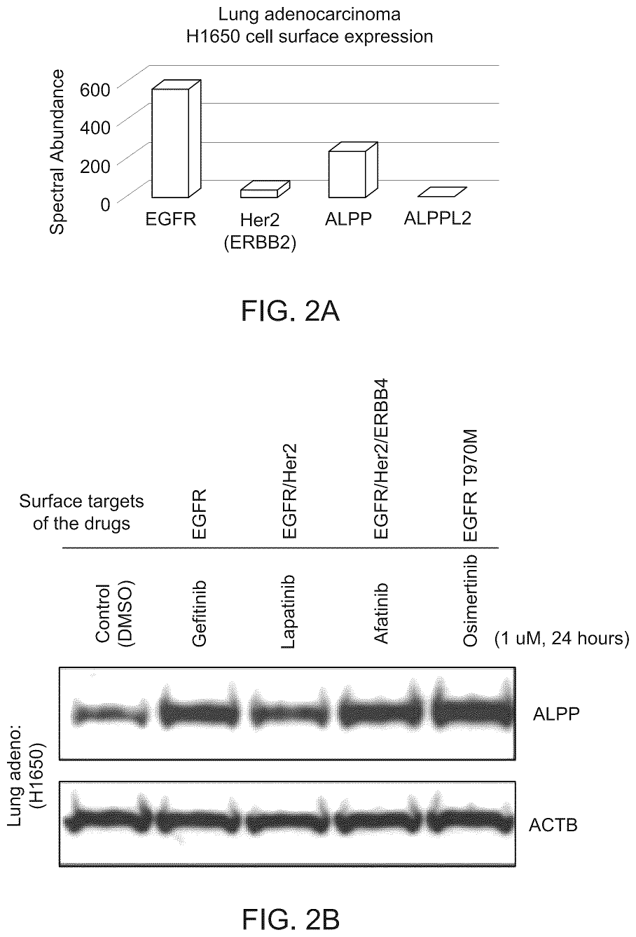

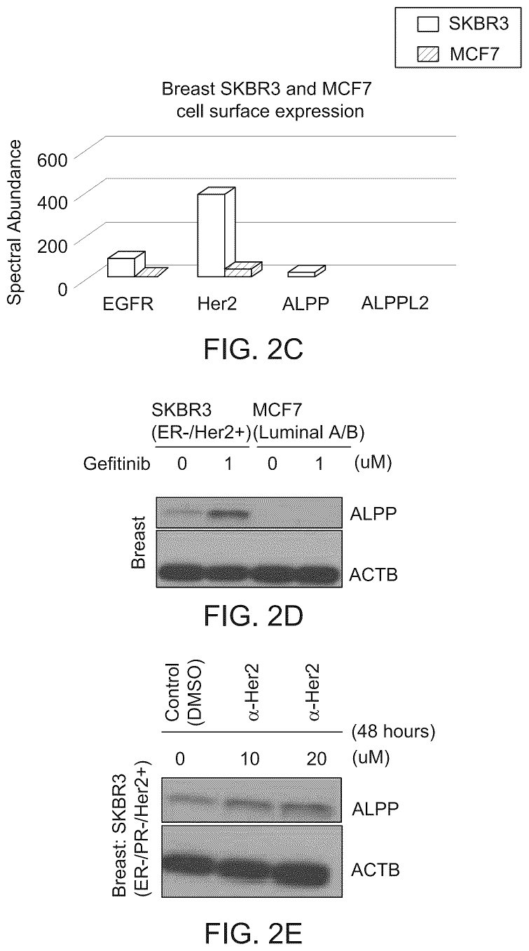

As a proof of concept, H1650 lung adenocarcinoma cells that co-express EGFR and ALPP were treated with several EGFR-targeting TKIs (FIG. 2B) and demonstrated upregulation of ALPP (FIG. 2A, B). The induction of ALPP by EGFR-TKI stimulation was also expandable to breast cancer, using SKBR3 cells (FIG. 2D). When basal ALPP expression was negative in MCF7 breast cancer cells, the receptor inhibition did not induce ALPP (FIG. 2C, D). The anti-HER-2 (ERBB2) monoclonal antibody stimulation against HER-2 expressed by SKBR3 cells similarly induced ALPP as compared to EGFR inhibition by small molecule TKIs, which confirmed that the concept of detecting and targeting cell surface receptors is broadly applicable (FIG. 2E).

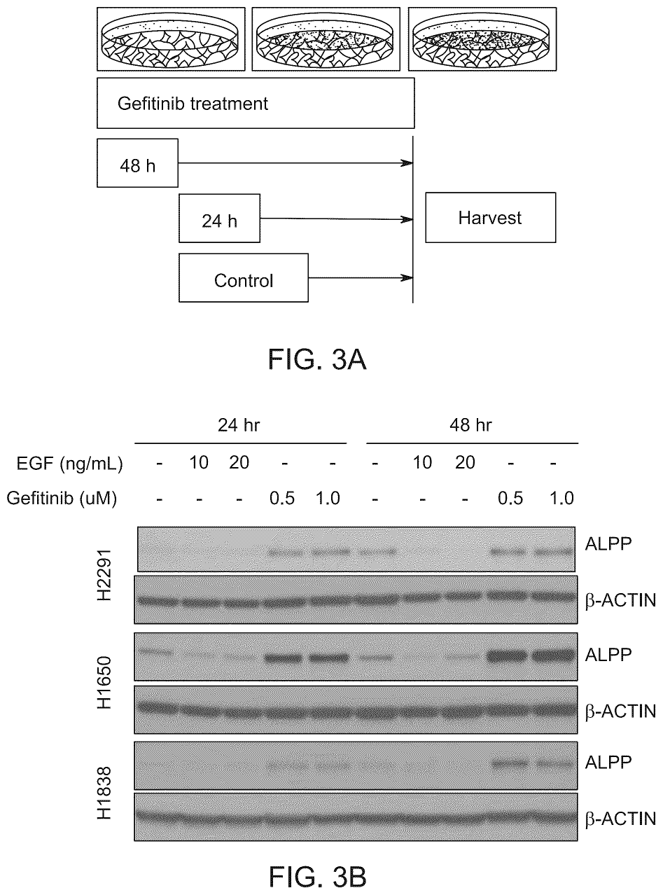

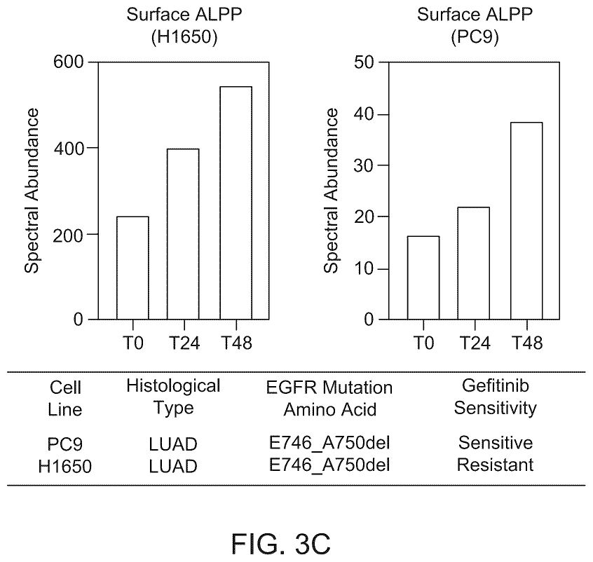

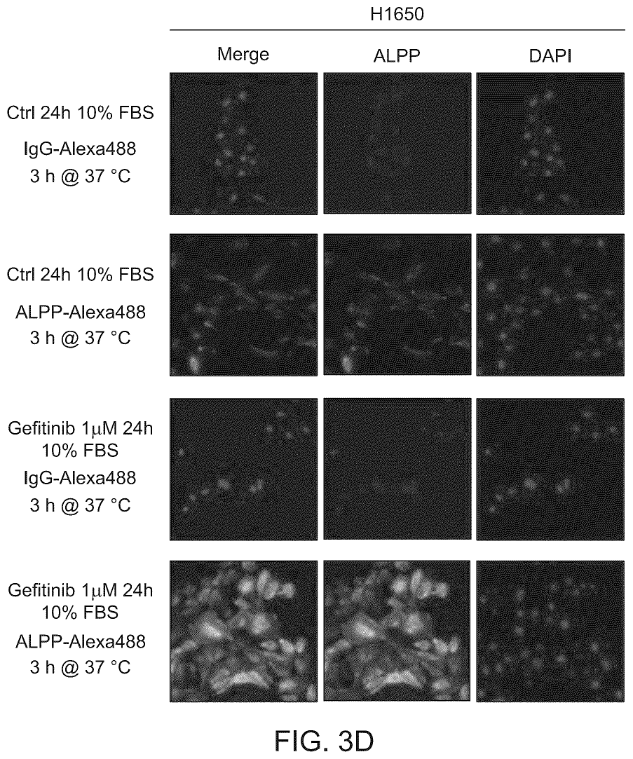

Targeting EGFR via an inhibitor, as in the case of Gefitinib in EGFR-overexpressing mutant lung adenocarcinoma (LUAD) cancer cell lines H1650 and PC9, resulted in pronounced upregulation of surface ALPP expression as confirmed by mass spectrometry and immunofluorescence (FIG. 3A-D). Immunoblots further confirmed ALPP upregulation following Gefitinib treatment, whereas stimulation of the EGFR with its natural ligand, EGF, reduced ALPP expression (FIG. 3B). Treatment of EGFR wild-type, KRAS mutant LUAD cell lines H2291 and H1838 with Gefitinib also increased ALPP expression, although to a lesser extent (FIG. 3B).

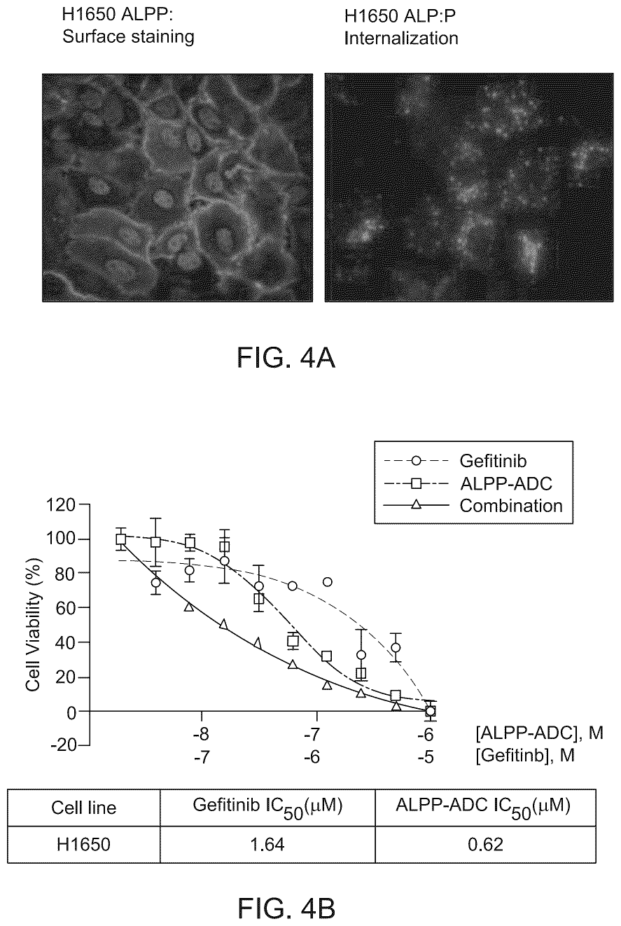

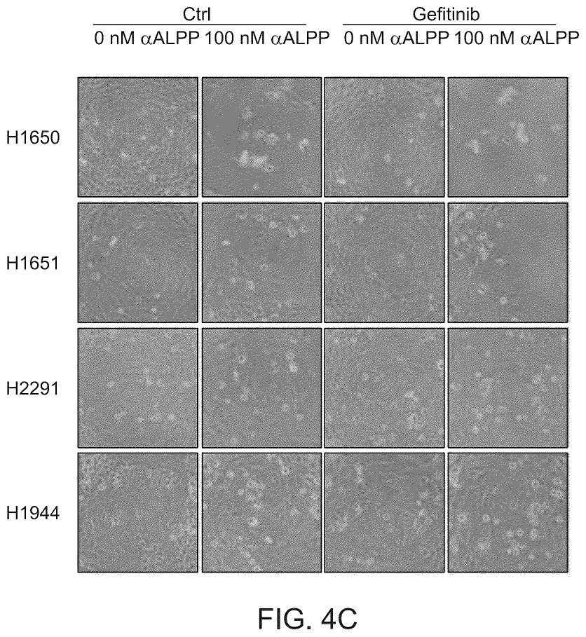

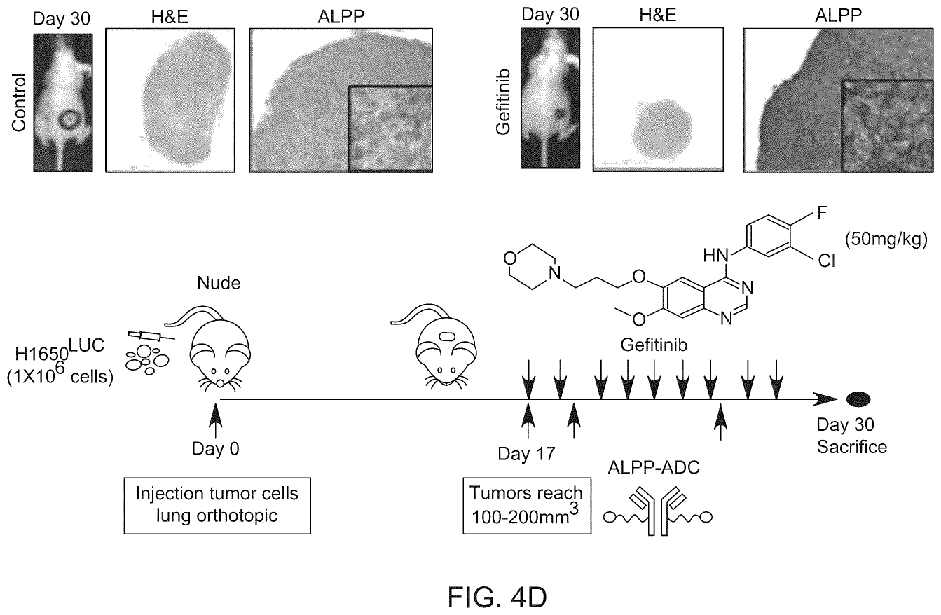

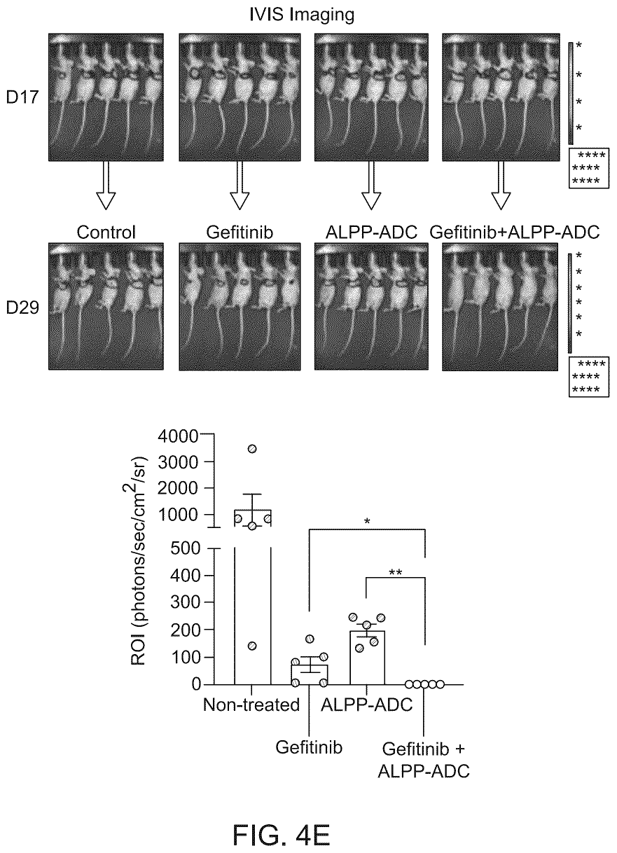

Targeting of cancer cell surface ALPP using anti-ALPP antibody showed internalization in EGFR mutant H1650 LUAD cells (FIG. 4A). Next, anti-ALPP antibodies conjugated with Monomethylauristatin F (MMAF) were synthesized. MMAF is a microtubule-disrupting agent inducing cell cycle arrest and apoptosis. Briefly, the drug-linker complex was Mc-vc-PAB-MMAF. The payload drug in this complex, MMAF, is an auristatin derivative that induces cell death by disruption of microtubule dynamics. The linker contains a valine-citrulline (Val-Cit) di-peptide module that is cleaved in the lysosome after antibody-drug conjugate (ADC) internalization, and a self-demolish PBA module to release MMAF in the native form. Mc-vc-PAB-MMAF is prepared by CBL and the purity of the compound is >95%. Mc-vc-PAB-MMAF is conjugated via the maleimide functional group to Cys residues on antibodies. General conjugation procedure: the antibody is partially reduced using either dithiothreitol (DTT), tris(2-carboxyethyl) phosphine (TCEP), or 5,5′-dithiobis (2-nitrobenzoic acid) (DTNB) to yield free inter-chain cysteine residues. Mc-vc-PAB-MMAF is conjugated to the antibody via these free cysteine residues through maleimide-thiol reaction. After conjugation, the remaining free Mc-vc-PAB-MMAF is quenched and all small-molecule species are removed by ultrafiltration to obtain the ADC.

The efficacy of Gefitinib and ALPP-MMAF-ADC were both tested alone and combination treatment for anti-cancer efficacy using H1650 LUAD cells in vitro. Combination treatment showed a synergistic effect and the highest anti-cancer efficacy (FIG. 4B-C). This strategy was additionally investigated using an orthotopic xenograft model of H1650 tumor-bearing mice, which confirmed that the combination of Gefitinib plus ALPP-MMAF-ADC yielded improved anti-cancer effects compared to control or either treatment alone (FIG. 4E).

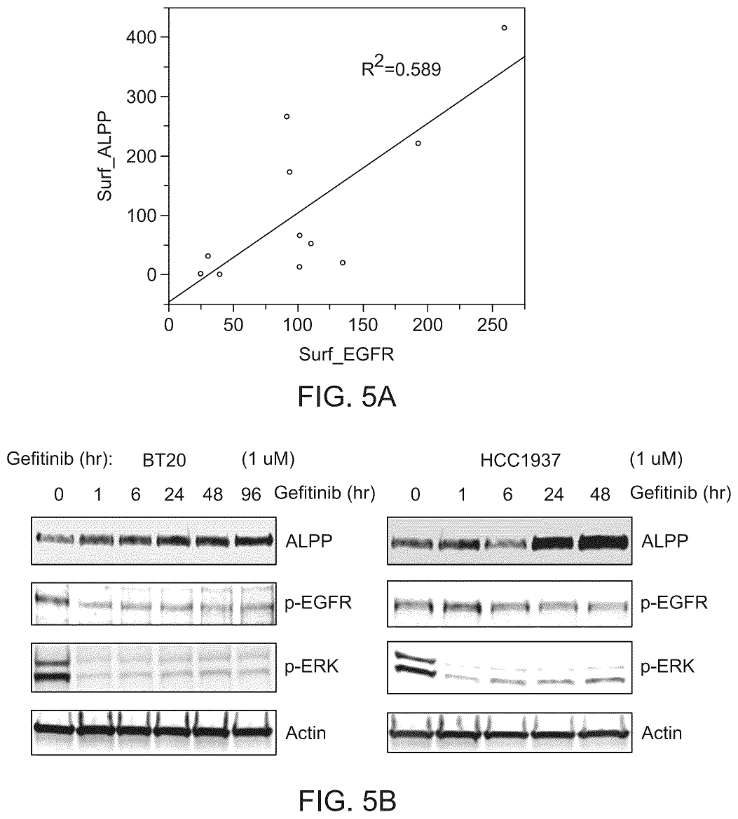

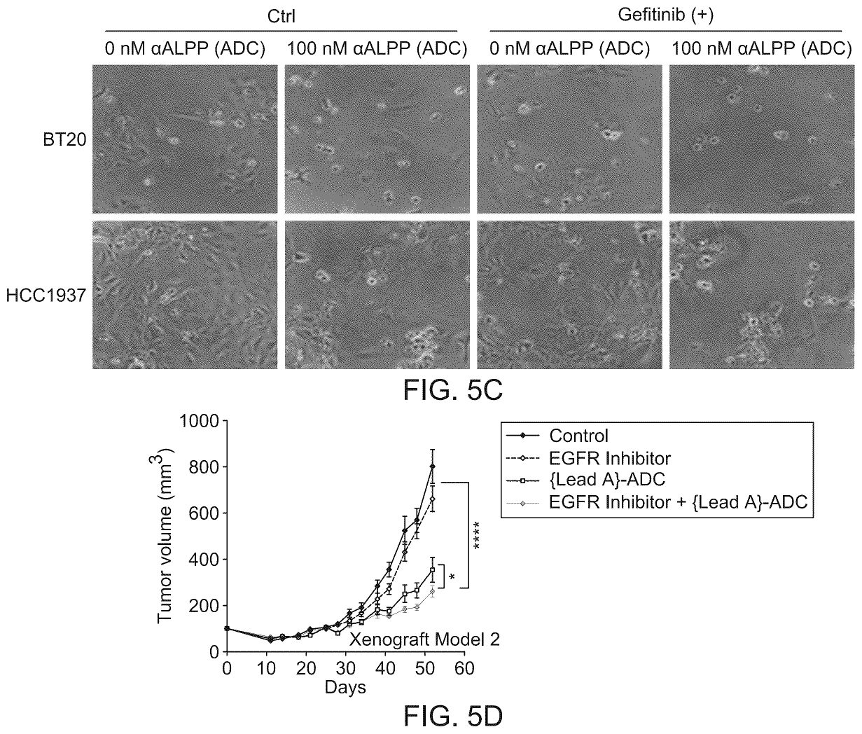

To demonstrate broader clinical relevance, additional tests were performed using triple-negative breast cancer (TNBC) cells, which similarly showed high surface ALPP expression that correlated with surface EGFR expression (FIG. 5A). Consistent with the findings in lung cancer, EGFR inhibition via Gefitinib increased ALPP expression in TNBC cell lines BT20 and HCC1937 (FIG. 5B). Combined treatment of Gefitinib plus ALPP-MMAF-ADC yielded strong anti-cancer effects in vitro and in an orthotopic xenograft model of TNBC (FIG. 5C-D).

Table 1 provides a list of cell-surface targets that are co-expressed with ALPP/ALPPL2 on lung adenocarcinoma and breast tumor that are known to be treated by drugs beyond EGFR and HER-2. Those surface target candidates had good overlap and expandable to various cancer types.

A diagnostic test that determines co-expression of cell surface targets and ALPP/ALPPL2 by means of IHC or other means would allow assessment of potential benefit from a combination of the inhibitors and ALPP targeted therapy for a broad spectrum of cancer types.

TABLE 1
Other Possible Targets Co-Expressing With ALPP/ALPP2 on Tumor Surface

| | | H1650 | PC9 | H2291 | | | SKBR3 |
|---|---|---|---|---|---|---|---|
| | | EGFR | EGFR | KRAS | HCC1937 | BT20 | ER-/ |
| Gene | Drugs | mut | mut | mut | TNBC | TNBC | Her2+ |
| CD22 | TKIs | | ID | | | ID | |
| CD40 | TKIs | ID | | ID | | ID | |
| CD46 | ADCs | ID | ID | ID | ID | ID | ID |
| CD74 | ADCs | | | | ID | | |
| DDR1 | TKIs | ID | ID | ID | ID | ID | ID |
| EFNB2 | TKIs | | ID | | ID | ID | |
| ENG | TKIs | ID | ID | ID | ID | ID | ID |
| EPHA2 | TKIs | ID | ID | ID | ID | ID | |
| EPHB2 | TKIs | ID | ID | ID | ID | ID | |
| EPHB4 | TKIs | ID | | ID | ID | ID | ID |
| ERBB3 | TKIs | | | | | ID | ID |
| FAS | monoAbs | ID | ID | ID | ID | | |
| FGFR2 | TKIs | | | | ID | | |
| ICAM1 | Other small | ID | ID | ID | ID | ID | |
| | molecule | | | | | | |
| | drugs | | | | | | |
| IGF1R | Other small | ID | ID | ID | | ID | |
| | molecule | | | | | | |
| | drugs | | | | | | |
| INSR | Other small | | ID | | ID | ID | |
| | molecule | | | | | | |
| | drugs | | | | | | |
| ITGA4 | monoAbs | ID | | ID | | | |
| ITGAV | TKIs | ID | ID | ID | ID | ID | ID |
| ITGB1 | monoAbs | ID | ID | ID | ID | ID | ID |
| ITGB3 | Fab | | | ID | | | |
| | fragment | | | | | | |
| MET | TKIs | ID | ID | ID | ID | ID | |
| MICB | monoAbs | ID | ID | ID | | | |
| MST1R | TKIs | ID | ID | ID | ID | ID | ID |
| NGFR | Other small | | | ID | ID | | |
| | molecule | | | | | | |
| | drugs | | | | | | |
| NRP1 | monoAbs | | | | | ID | |
| PTK7 | monoAbs | ID | ID | ID | ID | ID | |
| RET | TKIs | | | | | ID | |
| ROR1 | monoAbs | | ID | ID | | | |
| ROR2 | ADCs | ID | | ID | | | |
| SEMA4D | monoAbs | ID | | ID | ID | ID | |
| TNFRSF10A | monoAbs | ID | ID | | | | |
| TNFRSF10B | monoAbs | ID | ID | ID | | | |
| TYRO | TKIs | | | | | ID | |

TKI, Tyrosine kinase inhibitor;
ACD, Antibody-drug conjugate;
monoAb, Monoclonal antibody

### Example 2

Epidermal growth factor receptor (EGFR) tyrosine kinase inhibitor (TKI) therapy is considered the standard-of-care for lung cancers harboring EGFR activating mutations, but with limited performance. Placental alkaline phosphatase (ALPP) and related ALPPL2 are expressed in various solid tumors, including lung cancer. It was found that tumors with EGFR-activating mutations repressed ALPP expression, while EGFR TKI upregulated and sustained ALPP surface expression in lung adenocarcinoma (LUAD). EGFR inhibition resulted in dephosphorylation and activation of FoxO3a, a transcriptional regulator of ALPP contributing to its overexpression. In vitro evaluation of the combined modality of EGFR TKI plus ALPP antibody conjugated with monomethylauristatin F (ALPP-MMAF) resulted in significantly increased cell death in EGFR-mutant LUAD cell lines HCC827 and PC9 compared with either treatment alone. EGFR TKI also upregulated ALPP expression and enhanced targeted killing of drug-resistant LUAD cells. Gefitinib treatment in a gefitinib-resistant LUAD xenograft model upregulated expression of ALPP in tumor cells but not in normal tissues. Gefitinib plus ALPP-MMAF resulted in greater tumor shrinkage compared with gefitinib or ALPP-MMAF alone. These findings support a combination therapy involving an EGFR inhibitor together with an ALPP antibody-drug conjugate for LUAD.

Epidermal growth factor receptor (EGFR), a member of type 1 receptor tyrosine kinases, is activated by EGF and other ligands and transduces important cell growth signals. It is expressed in more than 60% of lung adenocarcinomas (LUAD) and mutated in 40%-60% of tumors in patients from South-East Asia and 10%-15% of Caucasians. EGFR TKI therapy has been used as the standard-of-care for lung cancer patients harboring EGFR-activating mutations. Despite the prolonged overall survival and high initial response to TKI treatment, the disease eventually progresses mainly due to insufficient killing of tumor cells and subsequent acquisition of drug resistance. TKI-refractory tumors also become resistant to most available drugs as well as to immune checkpoint inhibitors. There remains an unmet need for new treatment modalities for patients with EGFR activating mutations.

Placental alkaline phosphatase (ALPP, also known as PLAP) and ALPPL2 (also known as ALPG) are members of the alkaline phosphatase family that are exclusively expressed in the placenta and share 98% similarity in amino acid sequence. Several studies have documented expression of ALPP and ALPPL2 in various cancer types. To this end, assessment of ALPP protein levels in 12,381 tumors by immunohistochemistry showed strong expression of ALPP predominantly in seminoma, embryonal carcinoma of the testis, and yolk sac tumors, with reduced expression in other cancer types. In lung cancer, ALPP expression is relatively reduced and is limited to adenocarcinoma.

The restricted expression of ALPP and ALPPL2 in normal tissues and their accessibility on the cancer cell surface support their potential as therapeutic targets. To this end, a chemical library screen resulted in a selective and potent ALPP inhibitor which specifically bound to ALPP-positive tumors in vitro and targeted cervical cancer in a mouse model of the disease. A fluorescent derivative of the ALPP inhibitor functioned as a bispecific engager directing chimeric antigen receptor-T cells to fluorescein on ALPP-positive tumor cells for chimeric antigen receptor (CAR) T-cell mediated cancer killing. Targeting ALPP with a α-ALPP and α-CD3 bispecific antibody resulting in killing of ALPP-positive colorectal cancer cell lines. Expression of ALPP in colorectal cancer led to its consideration for CAR T-cell therapy. ALPP-CAR-T cells mediated potent cytotoxicity towards cancer cells, while the combination of ALPP-CAR-T cells with anti-PD-1, PD-L1, or LAG-3 checkpoint inhibitors further increased the therapeutic efficacy of CAR T-cells. Second-generation CAR T-cells with a fully humanized scFv against ALPP effectively killed ALPP-expressing HeLa cells. A clinical trial using α-ALPP CAR T cells for ovarian and endometrial cancer has been initiated and another clinical study evaluating the efficacy of ALPP/ALPPL2 antibody-drug conjugate in advanced solid tumors is ongoing.

In the current study, factors involved in regulating expression of ALPP in LUAD were investigated. ALPP expression in LUAD with EGFR activating mutations was significantly lower than in EGFR wildtype (WT) tumors and demonstrated that EGFR TKI substantially upregulated the expression of ALPP. FoxO3a was identified as an upstream transcriptional regulator of ALPP in the context of EGFR inhibition. Combination therapy of EGFR TKI plus ALPP-MMAF resulted in enhanced anti-cancer effects and prevented the formation of drug-resistant cell clones from EGFR TKI-sensitive LUAD cell lines HCC827 and PC9 in vitro. The combination therapy also resulted in enhanced anti-cancer response compared with gefitinib or ALPP-MMAF alone in a xenograft mouse model of LUAD.

### Cell Culture

Detailed information regarding the human cancer cell lines used in this study is provided in Table 4. Cells were cultured in RPMI 1640, Cat. #10-040-CV, Corning) supplemented with 10% inactivated fetal bovine serum (FBS, Cat. #16140-071, Gibco) and maintained at 37° C. in a humidified atmosphere with 5% CO₂.

### Antibodies, Chemicals, and Virus Strains

Detailed information regarding the antibodies, chemicals, and virus strains used in this study is provided in Table 5. ALPP antibody (Cat. #NB110-3638, Novus biologicals) was conjugated with monomethyl auristatin F (MMAF) by Creative Biolabs.

### Proteomic Analysis

ALPP and ALPPL2 expression analysis in whole cell extracts and in the surfaceome compartment was done by mass spectrometry as previously described¹⁹⁻²³. Each dataset was normalized to the total number of spectral counts for each compartment²⁴.

### FoxO3a Overexpression

The open reading frame of FoxO3a was inserted into pLOC-RFP vector and packed into lentivirus. Stable FoxO3a overexpression in LUAD cell lines HCC827 and H1650 was achieved by lentivirus infection and subsequent selection of blasticidin-resistant cells and overexpression of FoxO3a was verified by immunoblots.

### Immunofluorescence

The 1% paraformaldehyde fixed LUAD cells were stained with ALPP antibody (Cat. #NB110-3638, Novus biologicals) and Alexa488-conjugated secondary antibody. Images were acquired in z-series on a spinning-disk confocal system.

### Flow Cytometry

LUAD cells were stained with ALPP antibody (Cat. #NB110-3638, Novus Biologicals) and Alexa488-conjugated secondary antibody (Cat. #A11029, Invitrogen), followed by flow cytometry analysis on a LSRII flow cytometer (BD).

For flow cytometry-based cell cycle analysis, ethanol fixed cells stained in Hoechst 33342 (1 μg/ml, Cat. #62249, Invitrogen) followed by cell cycle analyses using a LSRII flow cytometer (BD).

### Immunohistochemistry (IHC) Analysis

Tissue microarrays for IHC staining of ALPP in this study comprised of 204 surgically resected lung cancer tumor specimens collected under an institutional review board protocol and archived as formalin-fixed, paraffin-embedded specimens in The University of Texas Specialized Program of Research Excellence thoracic tissue bank at The University of Texas MD Anderson Cancer Center. Patient characteristics for the analyzed cohort are provided in Table 3. A human normal tissue microarray for IHC staining of ALPP was obtained from Novus Biologicals (Cat. #NB110-3638). IHC staining was performed as previously described 25.

### Cell Viability Assay

Cell viability of LUAD cells was determined using the CellTiter 96 Aqueous One Solution Cell Proliferation Assay (MTS) kit (Cat. #G3580, Promega).

### Chromatin Immunoprecipitation (ChIP)

ChIP was performed in HCC827 and H1650 cells using Pierce Magnetic ChIP Kit (Cat. #26157, Thermo Scientific) following the manufacturer's instruction. The nuclei lysis was incubated with antibodies (Positive antibody: 10 μL; Negative antibody: 2 μL; FoxO3a antibody: 8 μL (Cat. #720128, Invitrogen)) overnight at 4° C. Subsequently, the DNA-protein complexes were enriched with Protein A/G magnetic Beads and DNA was recovered by the DNA Clean-up column. The relative abundance of genomic DNA fragments was determined using Sso Advanced Universal SYBR Green Supermix (Cat. #1725271, Bio-Rad) with the primers at forth in SEQ ID NOs: 1-10, shown in Table 2 below.

TABLE 2
Primers specific for the promoter region of
human ALPP gene

| | | SEQ ID |
|---|---|---|
| Primer | Sequence (5′-3′) | NO |
| ALPP-P1-F | CTACTGCCAACCACGGATGAATG | 1 |
| ALPP-P1-R | CAAGGGCTGTTGCCATATTTCC | 2 |
| ALPP-P2-F | TCGAAAAATAATTGTGGGGTGGC | 3 |
| ALPP-P2-R | GCTGAGTTAGGAAATTTCTGGGC | 4 |
| ALPP-P3-F | AAAAATAATTGTGGGGTGGCGG | 5 |
| ALPP-P3-R | CATTCCTTCCTCCTGCTTATTGC | 6 |
| ALPP-P4-F | CGTCCAAACATTAGTGTGAGTGC | 7 |
| ALPP-P4-R | CCCTTGCTCCATCATCTTACCTT | 8 |
| ALPP-P5-F | AAGGTAAGATGATGGAGCAAGGG | 9 |
| ALPP-P5-R | CTATATTTCCCAGAAGCTGGGGC | 10 |

Total RNA Extraction and Quantitative PCR (qPCR)

Total RNA was extracted using the RNeasy Mini Kit (Cat. #74104, QIAGEN). Reverse transcription of total RNA was performed using the High-Capacity Reverse Transcriptase Kit (Cat. #4368813, Applied Biosystems). TaqMan Universal Master Mix II (Cat. #4440040, Applied Biosystems) was used for analyzing ALPP expression. Probe for ALPP was purchased from Thermo Fisher Scientific (Hs03046558_s1).

### Immunoblotting

Total proteins were extracted in RIPA buffer (Cat. #89901, Pierce) containing complete protease inhibitor cocktails (Cat. #04693116001, Roche) and phosSTOP (Cat. #04906837001, Roche) Information for antibodies is provided in Table 4.

### Antibody Internalization Assay

Antibody internalization was assessed using the pHrodo™ iFL Red Microscale Protein Labeling Kit (Cat. #P36014, Thermo Fisher) following the manufacturer's instructions. Briefly, 100 μg of anti-ALPP antibody (Cat. #MAB59051, R&D systems) in 100 μl PBS was mixed with 10 μl of 1 M sodium bicarbonate and 5 μl of 2 mM pHrodo followed by incubation for 30 minutes at room temperature. The pHrodo-conjugated anti-ALPP antibody was subsequently purified using the gel resin. Cells were washed twice with PBS and incubated with pHrodo-conjugated anti-ALPP antibody (10 μg/ml) for 24 hours, followed by fixation in 2% paraformaldehyde, two washes with PBS, and mounting with DAPI. Images were captured using a fluorescent microscope.

### In-Vivo Studies

Animal experiment protocols were approved by The University of Texas MD Anderson Cancer Center IRB and in accordance with the Guidelines for the Care and Use of Laboratory Animals published by the NIH (Bethesda, MD). BALB/c nude mice (Cat. #194, Charles River) were housed in specific pathogen free facilities. For orthotopic xenograft models of LUAD, a total of 1×10⁶ cells H1650 cell lines expressing firefly luciferase were suspended in 50 μl 50% Matrigel Matrix (Corning)/Opti-MEM media and injected into the lung of 8- to 10-week-old nude mice. For gefitinib treatment, mice received gefitinib (50 mg/kg) via oral gavage daily for 10 days starting from Day 17 post cancer cell inoculation. For the gefitinib/ALPP-ADC combinational treatment, mice received additional ALPP antibody conjugated with MMAF (ALPP-MMAF) (5 mg/kg, i.v.) on Day 19 and Day 26. The tumor growth was monitored twice a week over a period of 5 weeks using the Xenogeny In Vivo Imaging System (IVIS, Alameda, CA). Mice were euthanized at Day 30 and the tumors were harvested and processed for routine histological and immunohistochemical analyses.

### Gene Set Enrichment Analysis (GESA)

To perform GESA, the “ReactomePA”, “clusterProfiler” packages in R and GSEA dataset (gsea-msigdb.org/gsea/index.jsp) was used to calculate the signal enrichment score with a significance threshold of FDR q value <0.05^{26,27}. The “enrichplot” package was applied for the visualization of the enrichment result.

Single-cell RNA-sequencing (scRNA-seq) scRNA-seq data was retrieved from GEO dataset (GSE150949)²⁸. The Seurat package (version 4.3.0) implemented in R statistical software (version 4.3) (https://www.r-project.org/) was used for the pre-processing, principal component analysis (PCA) based-dimension reduction, t-distributed stochastic neighbor embedding (t-SNE) to visualize the cell clusters from different treatment groups. The expression and percentage of ALPP was calculated accordingly. Furthermore, the cells were classified into S/G2M or G1 phase and cell cycle score was calculated through the function CellCycleScoring under the Seurat package²⁹.

### Statistical Analysis

For continuous variables, statistical significance was determined by 2-sided Student T-Test unless otherwise specified. For categorical variables, statistical significance was determined by Fisher's Exact Test for two class categorical comparisons or χ² test for trend for multiple categorical variables. Figures were generated in GraphPad Prism Version 9.0.0.

### Results

### ALPP and ALPPL2 Expression in LUAD

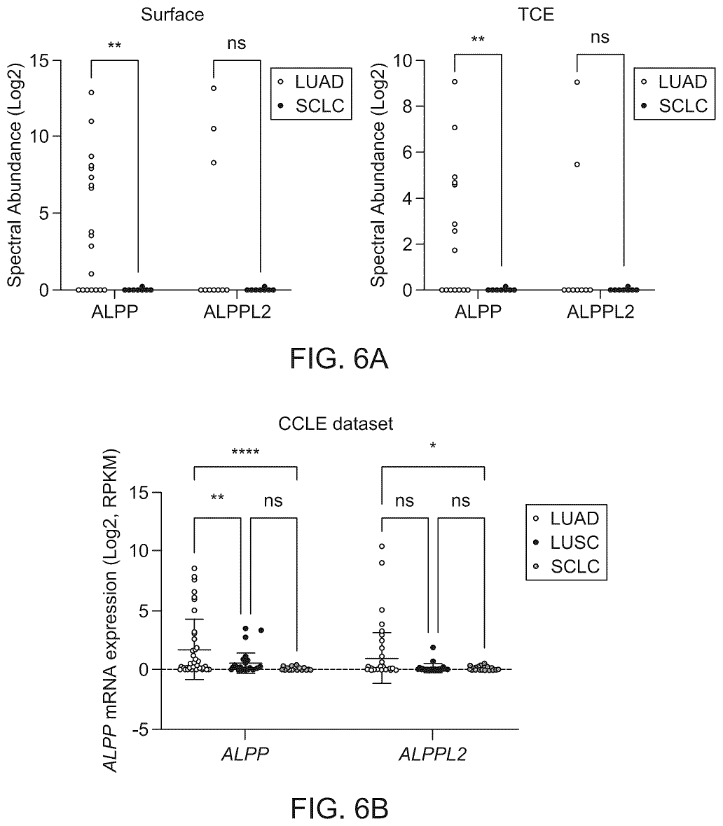

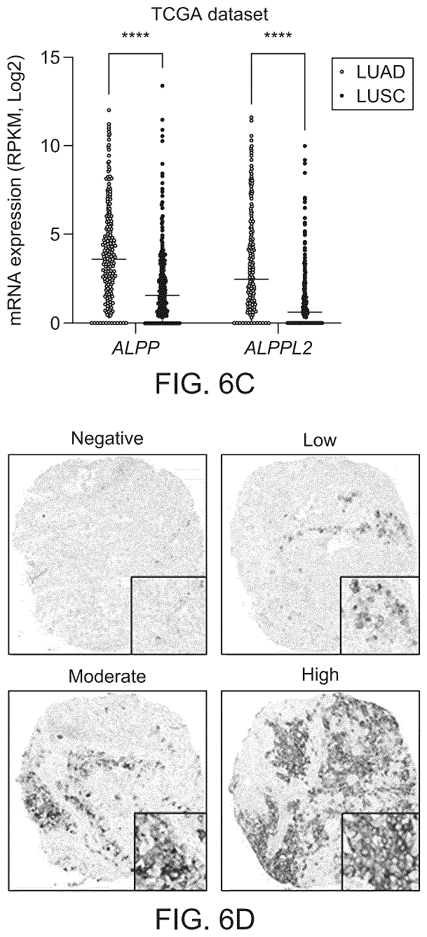

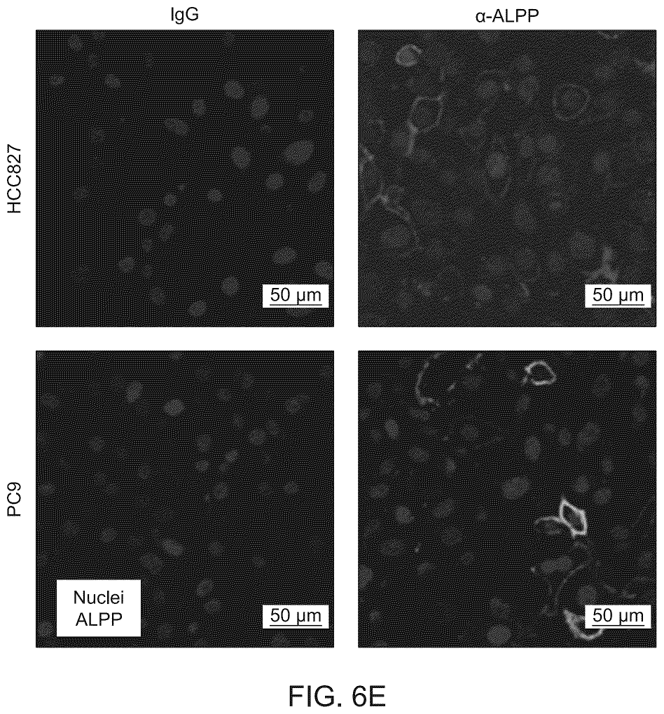

To assess the expression of ALPP and ALPPL2 in lung cancer, ALPP and ALPPL2 protein levels were evaluated in the whole cell extract (WCE) and the surface compartment of 49 lung adenocarcinoma (LUAD) and 22 small cell lung cancer (SCLC) cell lines, which revealed ALPP, and to a lesser extent ALPPL2, were predominantly expressed in LUAD compared with SCLC. (FIG. 6A and Table 4). Assessment of their mRNA expression levels in 48 LUAD, 24 lung squamous carcinoma (LUSC) and 50 SCLC cell lines from the Cancer Cell Line Encyclopedia (CCLE) similarly revealed ALPP to be expressed predominantly in LUAD, concordant with the proteomic results (FIG. 6B). Analysis of ALPP and ALPPL2 mRNA expression in LUAD and LUSC in The Cancer Genome Atlas (TCGA) also revealed higher expression in LUAD compared with LUSC (FIG. 6C).

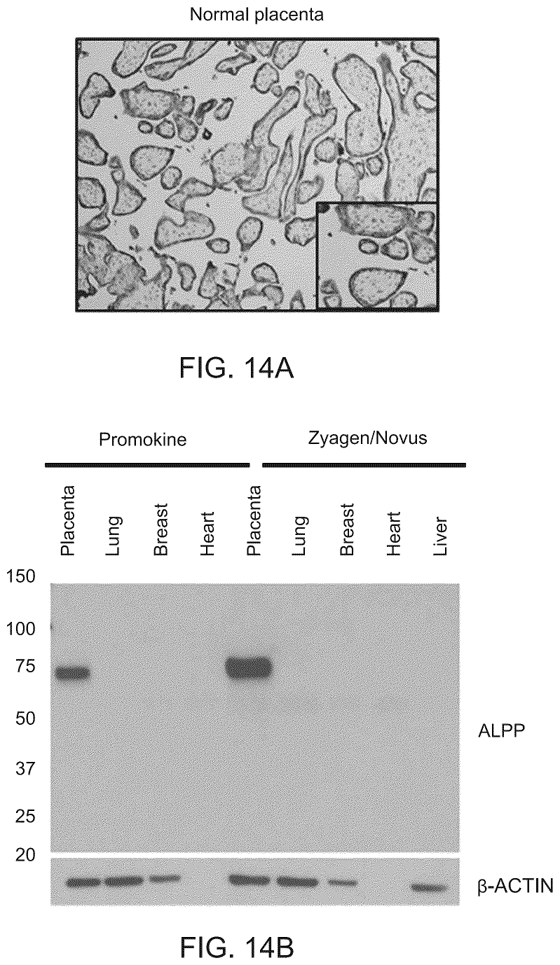

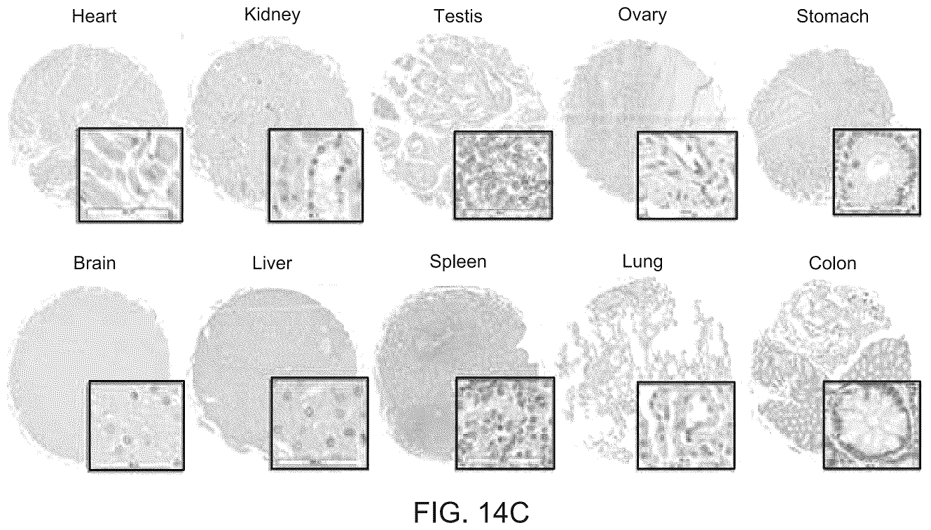

Since the expression level of ALPP is higher than ALPPL2 in LUAD (FIG. 6A-FIG. 6C), focused was placed on ALPP as a potential therapeutic target. Using a tissue microarray (TMA) consisting of 140 LUAD and 64 LUSC tumors, ALPP expression was assessed by immunohistochemistry (IHC). Of the 204 tumors, 37 (18.1%) stained positive for cytosolic ALPP and 23 (11.3%) stained positive for membranous ALPP (FIG. 6D). It was also confirmed that ALPP surface expression by immunofluorescence in EGFR-mutant HCC827 and PC9 LUAD cells (FIG. 6E). ALPP membrane staining positivity was more frequent in LUAD tumors (21 out of 140) than in LUSC tumors (2 out of 64; Fisher's exact test p-value: 0.015), consistent with prior reports. ALPP membrane staining positivity was associated with smoking status (χ² test for trend p-value: 0.0001) (Table 3). There was no statistical evidence for associations between tumoral ALPP membrane staining positivity and sex or stage (Table 3). Expression of ALPP in normal human tissues was assessed. Except for placental and testis tissues, ALPP protein expression was negative in other tissues, including the lung (FIG. 14A-FIG. 14C).

TABLE 3
ALPP Membrane Staining Positivity in Tissue Microarray
of Lung Adenocarcinoma and Squamous Cell Carcinoma

| | Membrane Staining | | |
|---|---|---|---|
| | ALPP | ALPP | |
| Variable | Positive | Negative | P-value† |
| Total | 23 | 181 | |
| Sex, N (%) | | | |
| Male | 8 (34.8) | 98 (54.1) | 0.12 |
| Female | 15 (65.2) | 83 (45.9) | |
| Smoking Status, N (%) | | | |
| Current | 14 (60.9) | 49 (27.1) | 0.0001 |
| Former | 7 (30.4) | 51 (28.2) | |
| Never | 0 (0) | 17 (9.4) | |
| Unknown | 2 (8.7) | 64 (35.4) | |
| Subtype, N (%) | | | |
| Adenocarcinoma | 21 (91.3) | 119 (65.7) | 0.015 |
| Squamous Cell Carcinoma | 2 (8.7) | 62 (34.3) | |
| Stage, N (%) | | | |
| I + II | 15 (65.2) | 125 (69.1) | 0.81 |
| III + IV | 8 (34.8) | 56 (30.9) | |
| Neoadjuvant Therapy, N (%) | | | |
| Yes | 1 (4.3) | 32 (17.7) | 0.10 |
| No | 22 (95.7) | 148 (81.8) | |

Statistical significance was determined by Fisher's Exact Test for two class categorical comparisons or χ² test for trend for multiple categorical variables. 2-sided P-values are reported.
Abbreviations: wt—wildtype.

### EGFR TKI Upregulates ALPP Expression in LUAD Cells

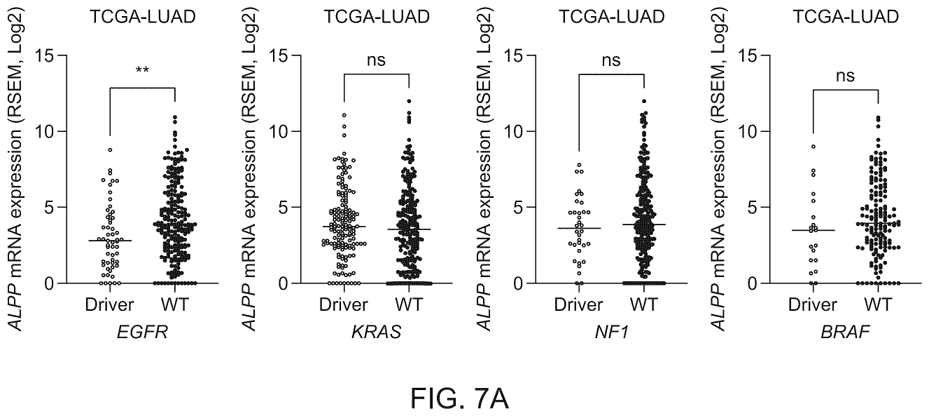

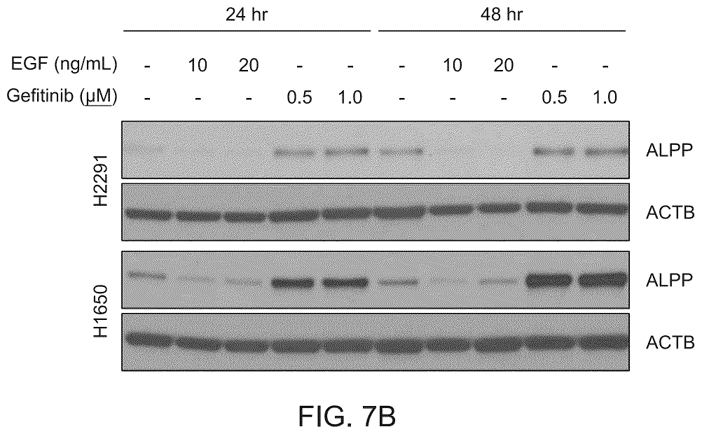

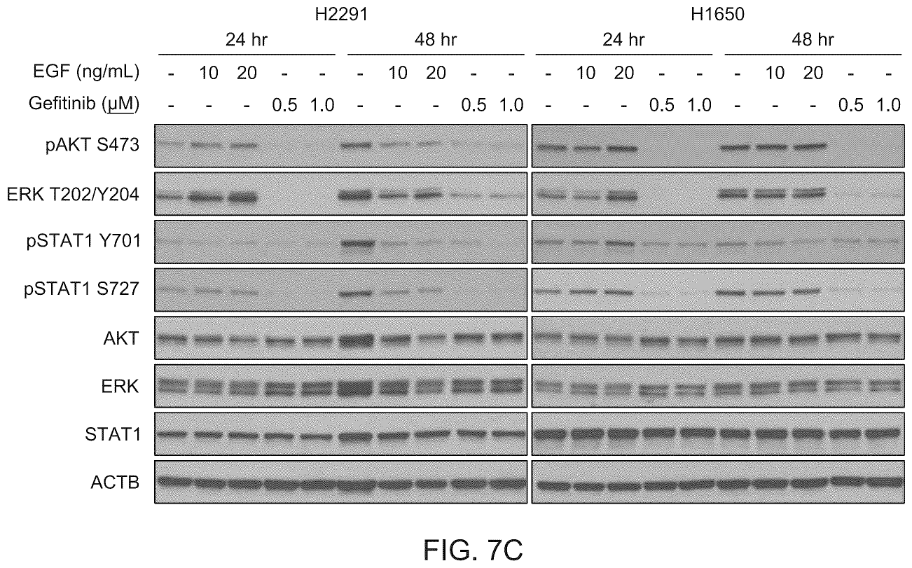

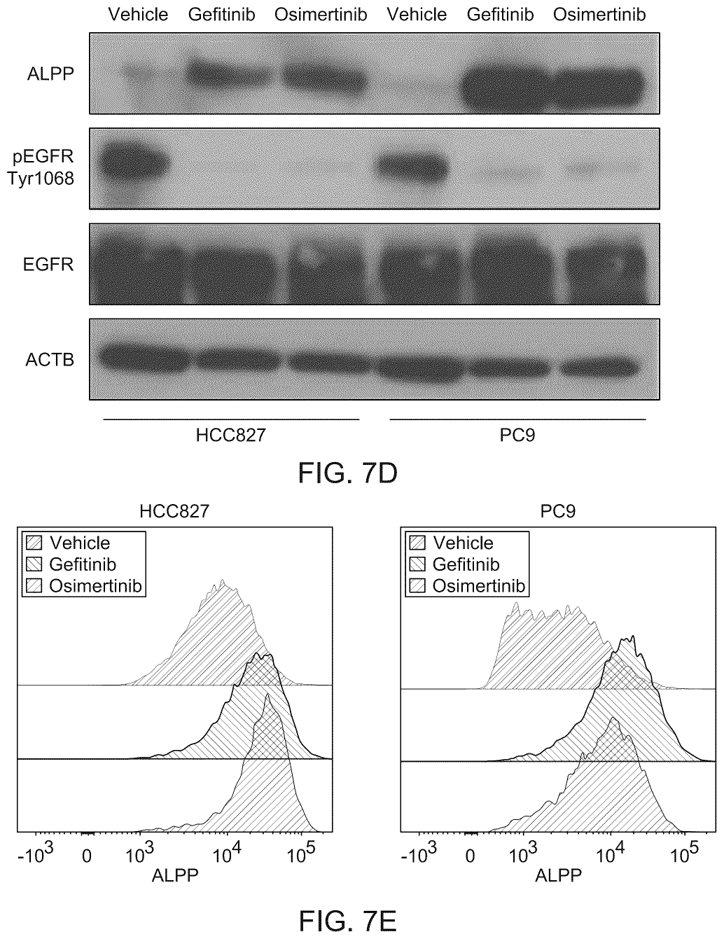

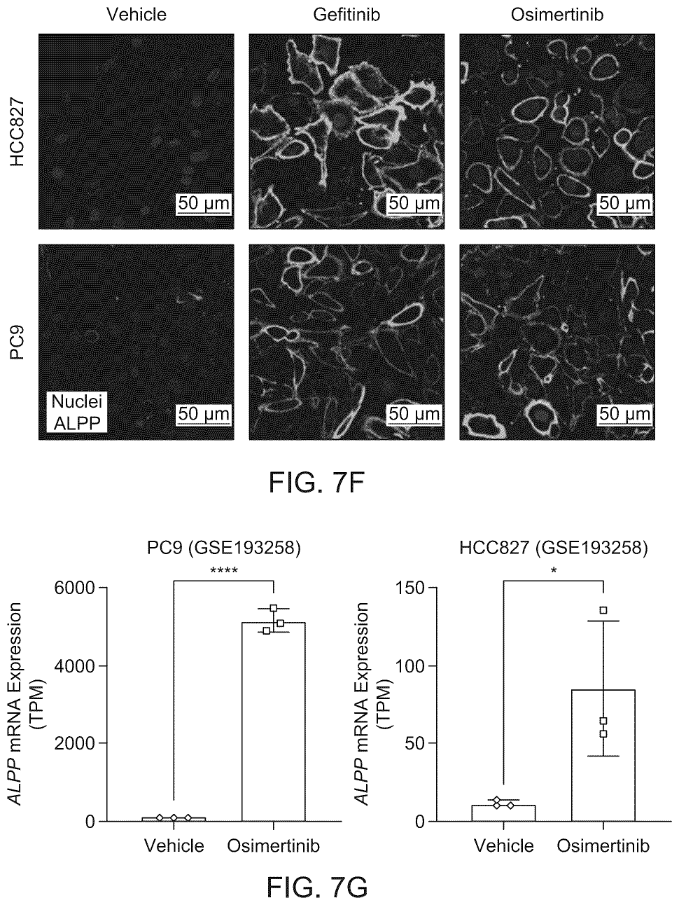

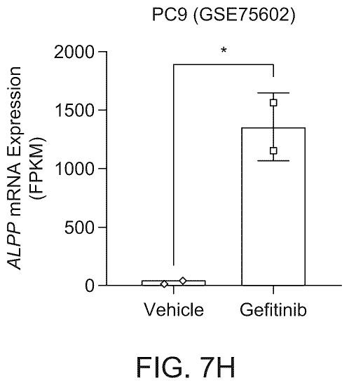

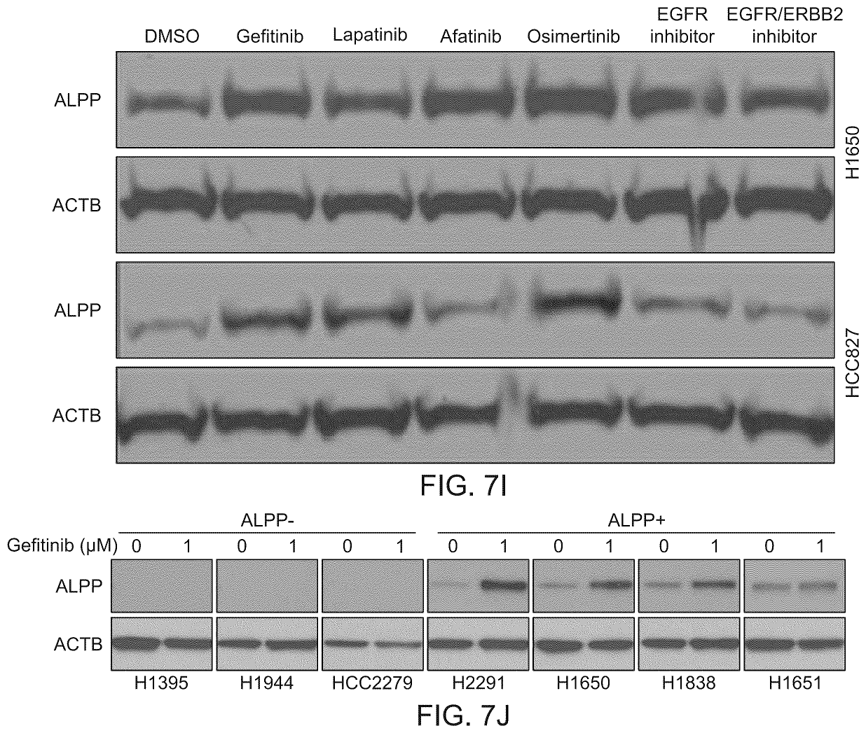

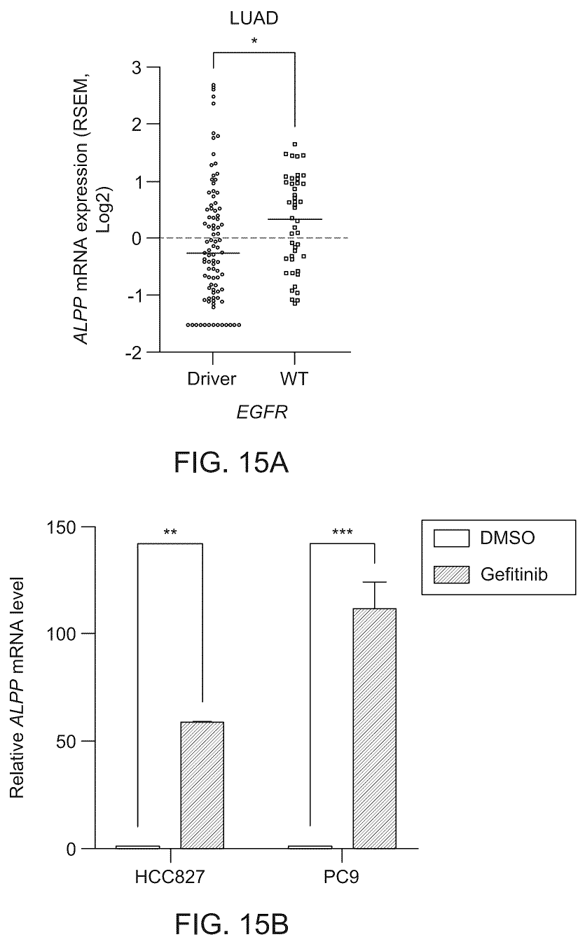

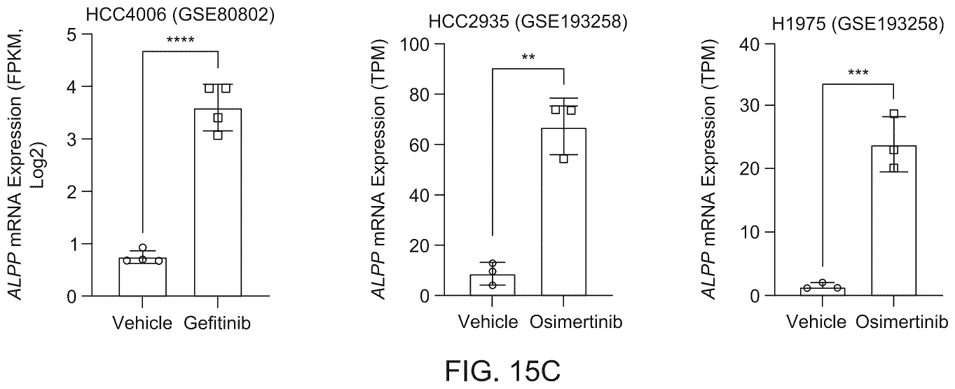

The relatively low expression of ALPP in LUAD may limit its utility as a therapeutic target. Activation of oncogenes commonly led to unchecked cell proliferation, whereas previous studies have reported an inverse association between ALPP expression and cell proliferation. To investigate whether the oncogenes contributed to repressed ALPP expression, ALPP gene expression in LUAD was compared with WT and driver mutations of EGFR, KRAS, NF1, and BRAF. LUAD with EGFR driver mutations expressed reduced ALPP compared with EGFR-WT tumors in TCGA dataset (FIG. 7A), which is consistent with findings from an independent cohort of 181 east Asians with LUAD (FIG. 15A). To test if the effect of EGFR signaling on ALPP expression depends on its activating mutation status, gefitinib-resistant EGFR-mutant H1650 and EGFR-WT H2291 LUAD cells were cultured in growth medium with or without epidermal growth factor (EGF), the endogenous ligand of EGFR. ALPP expression was drastically reduced in both cell lines cultured in EGF-containing growth medium (FIG. 7B). EGF induced AKT phosphorylation at serine 473 (Ser473), and ERK phosphorylation at threonine 202 (T202) and tyrosine 204 (Y204) residues, whereas EGFR TKI gefitinib treatment repressed phosphorylation of AKT and ERK (FIG. 7C). In addition, EGFR TKI-sensitive EGFR-mutant HCC827 and PC9 cells treated with gefitinib or osimertinib exhibited approximate 60- and 110-fold increases in ALPP expression (FIG. 7D and FIG. 15B). Immunofluorescence staining and flow cytometry analysis demonstrated enriched cell surface expression of ALPP upon EGFR inhibition (FIG. 7E and FIG. 7F). Expression levels of ALPP from independent datasets confirmed concordant elevation upon EGFR TKI treatment (FIG. 7G-FIG. 7H and FIG. 15C).

To determine whether other EGFR inhibitors induce ALPP expression, treatment of EGFR-mutant H1650 and HCC827 LUAD cells with inhibitors including lapatinib, afatinib, and osimertinib yielded similar results (FIG. 7I). Upregulation of ALPP with EGFR inhibitors was also observed in EGFR-WT LUAD cell lines H2291, H1838, and H1651 (FIG. 7J). However, ALPP non-expressing cell lines H1395, H1944, and HCC2279 did not upregulate ALPP in response to gefitinib treatment, whereas ALPP-positive LUAD cell lines H2291, H1650, H1838, and H1651 exhibited elevated ALPP levels (FIG. 7J).

### FoxO3a is a Transcriptional Regulator of ALPP

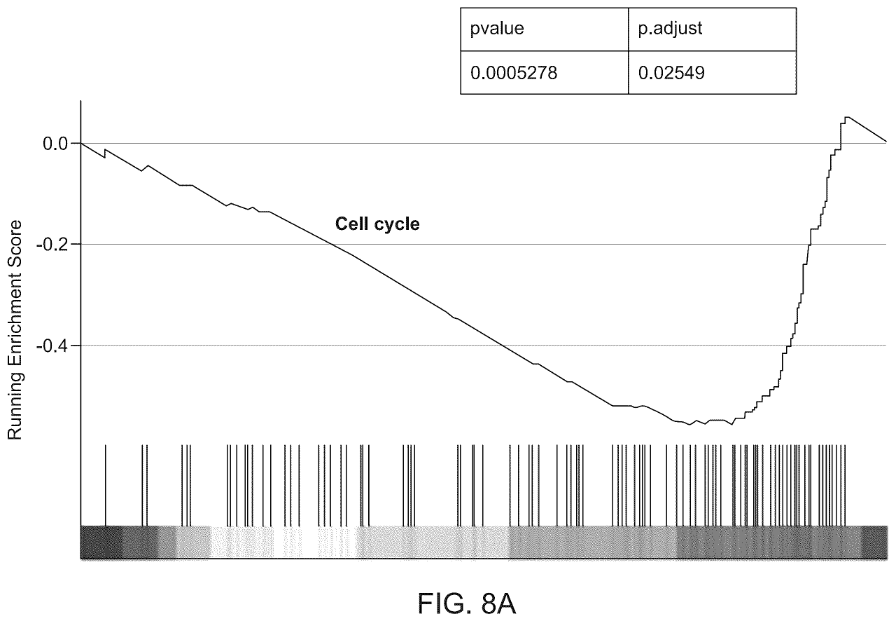

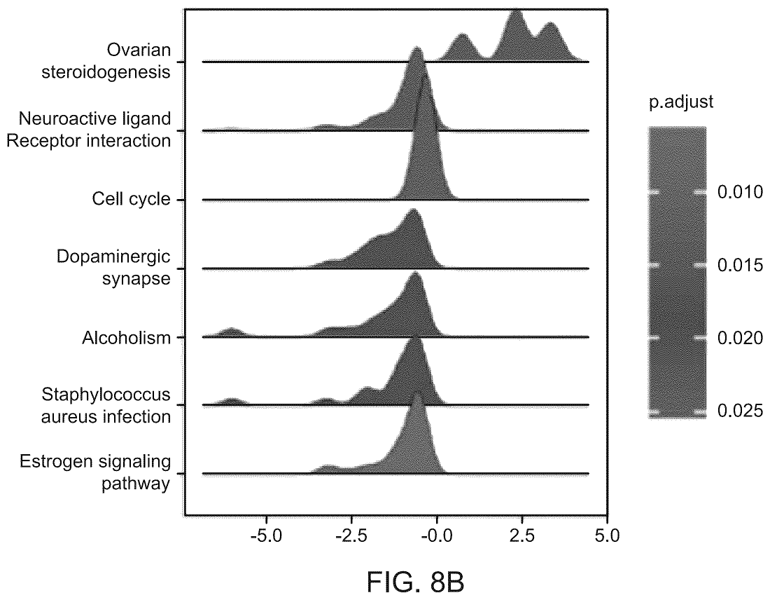

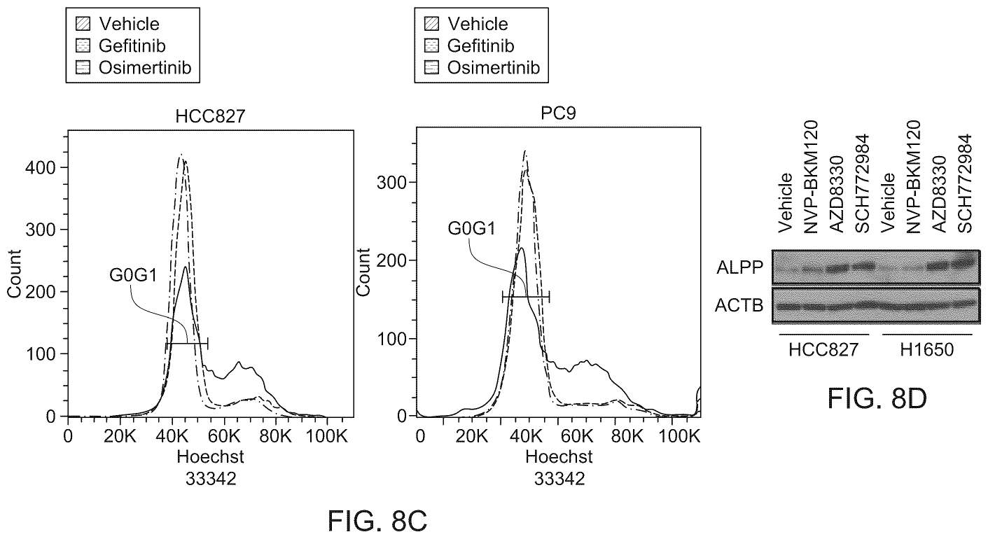

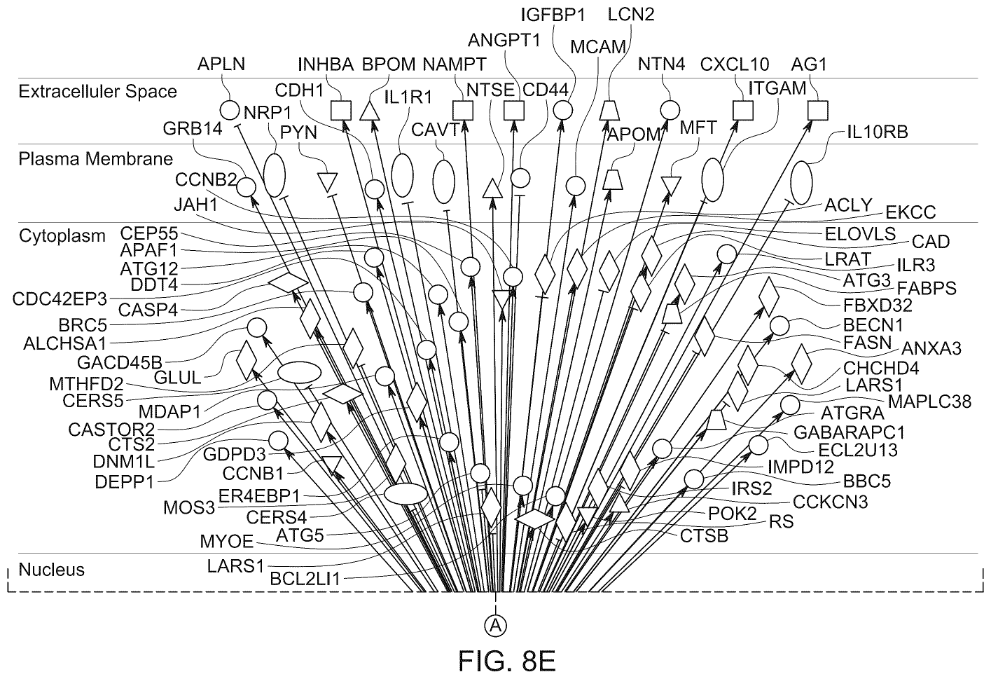

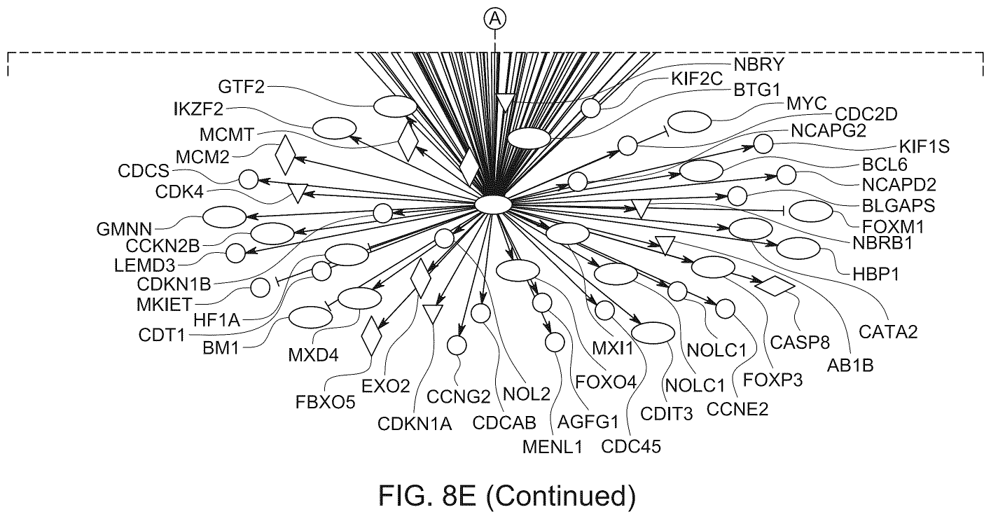

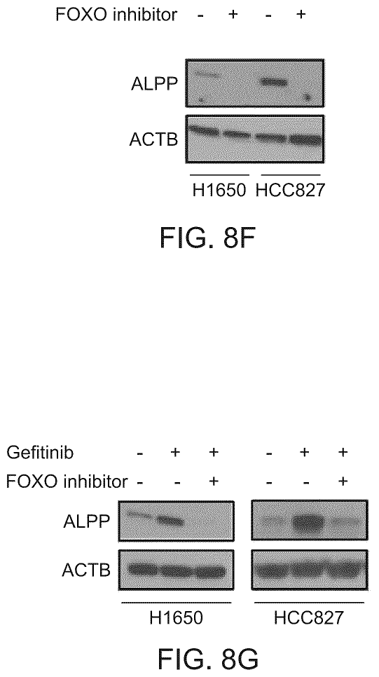

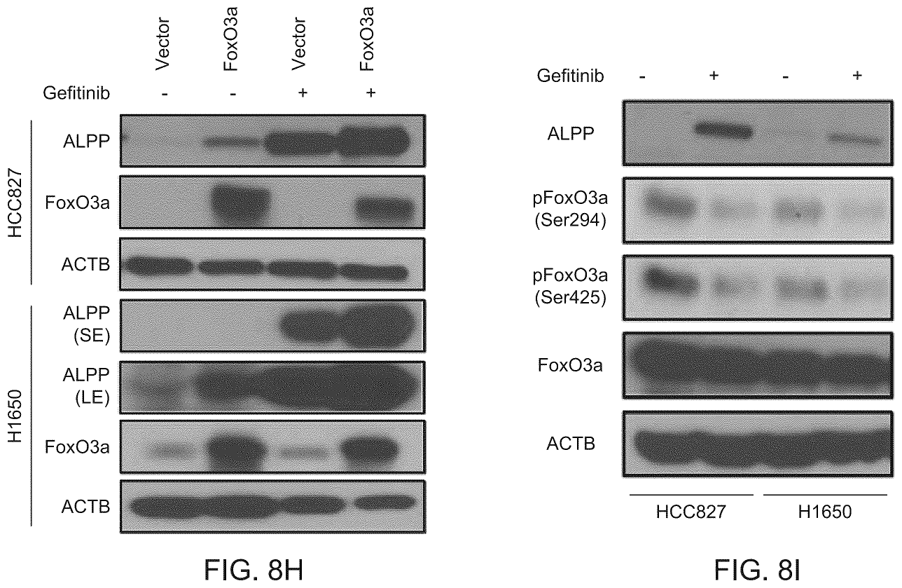

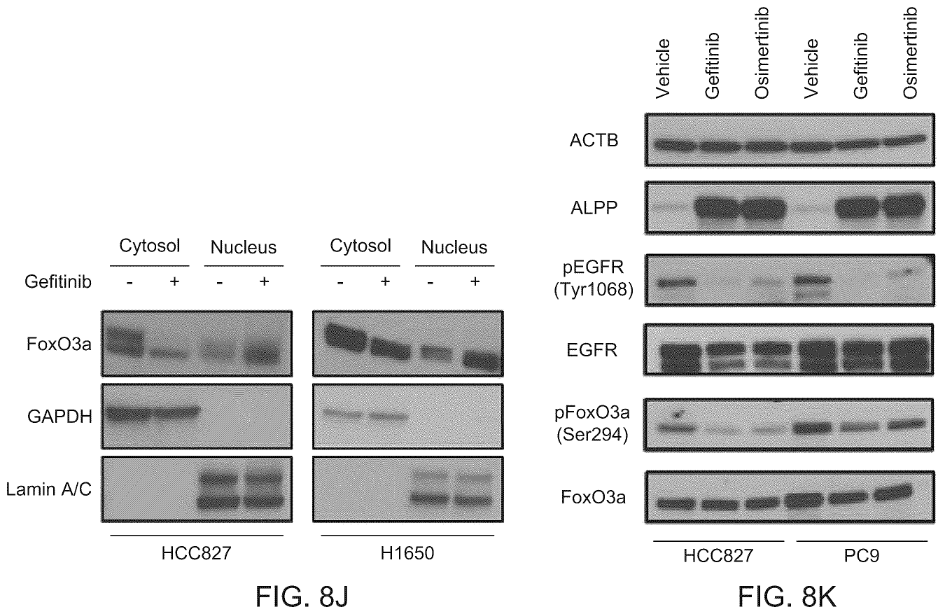

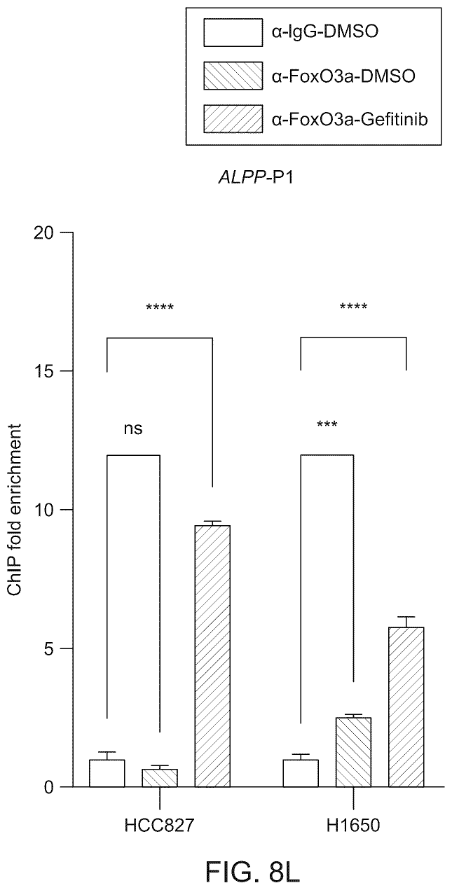

Given the inverse association between ALPP expression and cell proliferation, GESA analysis of the TCGA LUAD dataset confirmed an inverse correlation of ALPP expression with cell cycle (FIG. 8A). KEGG pathway analysis also revealed cell cycle as one of the top pathways inversely correlated with ALPP expression (FIG. 8B). Gefitinib or osimertinib treatment of HCC827 and PC9 cells revealed statistically significantly increased cell cycle arrest in G0/G1 (FIG. 8C).

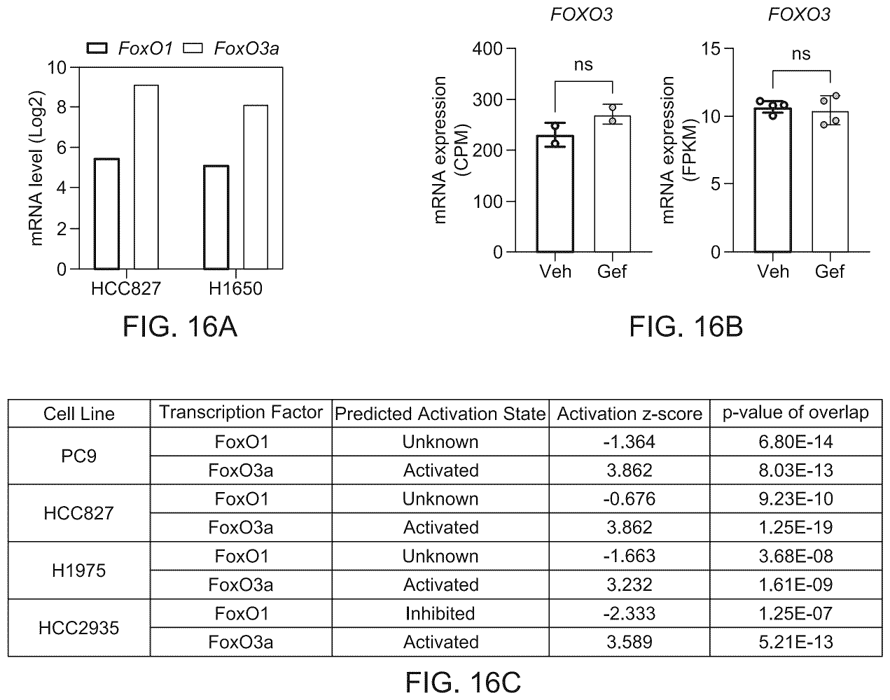

It was next tested whether signaling pathways that directly downstream of EGFR are involved in ALPP upregulation. Small molecule inhibition of PI3K/AKT (NVP-BKM120) or MEK/ERK (AZD8330, SCH772984) signaling in HCC827 and H1650 LUAD cells resulted in a drastic induction of ALPP expression (FIG. 8D). FoxO family transcription factors are involved in cell cycle regulation and are regulated by PI3K/AKT and MEK/ERK signaling. At the mRNA level, EGFR-mutant H1650 and HCC827 LUAD cells expressed low FoxO1 but appreciable FoxO3a (FIG. 16A). The transcription of FOXO3A was not affected by gefitinib treatment (FIG. 16B). Ingenuity Pathway Analysis of upstream regulators revealed concordant activation of FoxO3a, but not FoxO1, across 4 LUAD cell lines treated with osimertinib (FIG. 8E and FIG. 16C). Phosphorylation of FoxO3a at Ser294 and Ser425 leads to nuclear export of FoxO3a and cytosolic retention, resulting in loss of transcriptional activity and degradation. Inhibition of FoxO3a using the small molecule inhibitor AS1842856 diminished ALPP protein expression and blunted gefitinib-mediated ALPP upregulation in LUAD cells (FIG. 8F-FIG. 8G). Inversely, overexpression of FoxO3a in both H1650 and HCC827 cell lines resulted in upregulation of basal ALPP expression, which was further potentiated when respective cancer cells were treated with gefitinib (FIG. 8H). Treatment of EGFR-mutant H1650 and HCC827 LUAD cells with gefitinib repressed phosphorylation of FoxO3a at Ser294 and Ser425 residues, reduced cytosolic FoxO3a and promoted its nuclear translocation, suggesting transcriptional activation (FIG. 8I-FIG. 8J). Osimertinib also suppressed the phosphorylation of EGFR and FoxO3a, and increased ALPP expression (FIG. 8K).

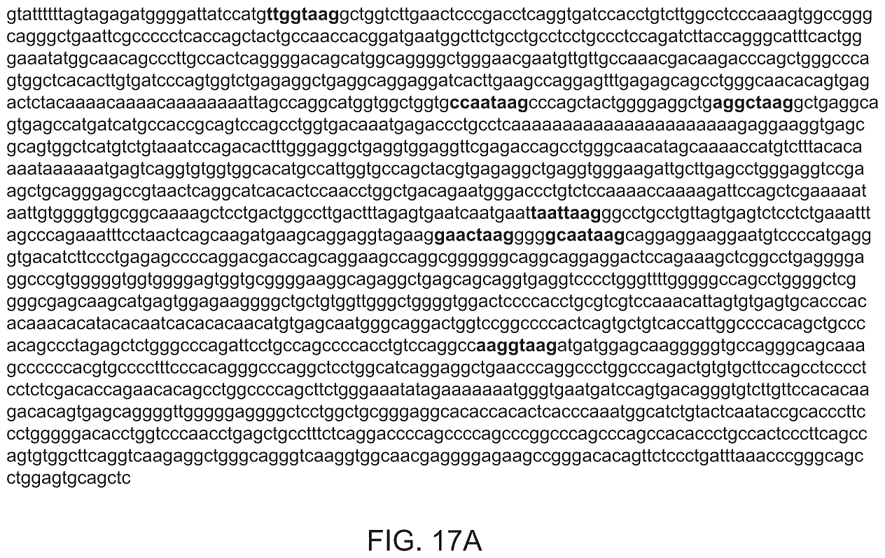

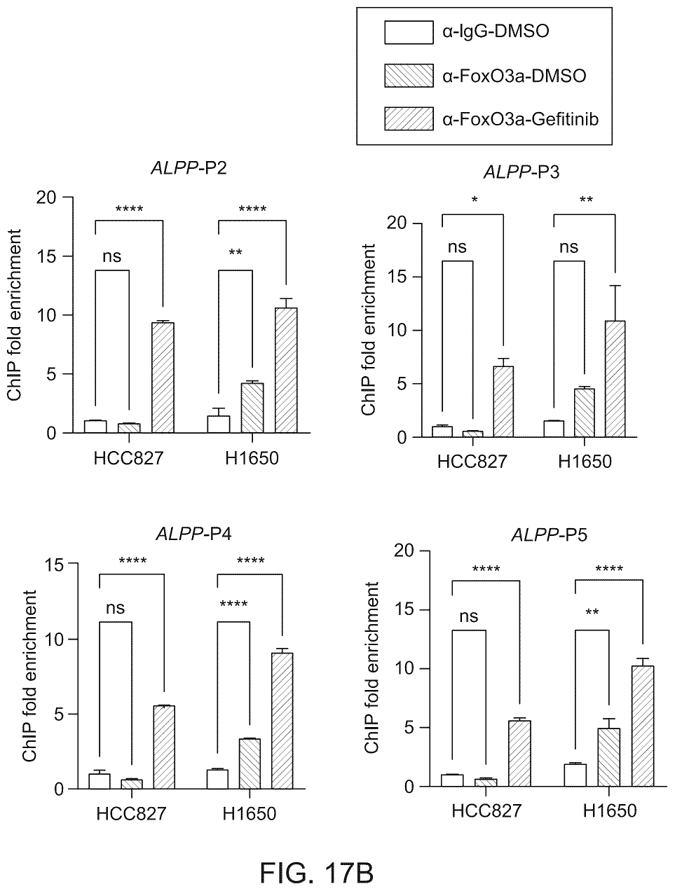

To elucidate whether FoxO3a was a transcriptional regulator of ALPP, the online tool PROMO was first used for in silico prediction of putative transcription factor(s) for ALPP. Seven potential binding sites for FoxO3a were identified within the 2 kb promoter region upstream of ALPP gene (FIG. 17A). ChIP-qPCR was then performed and confirmed that gefitinib treatment induced FoxO3a binding to ALPP promoter sequences, which led to concomitant increases in ALPP mRNA levels in both H1650 and HCC827 LUAD cell lines (FIG. 8L and FIG. 17B). Taken together, these data demonstrated that FoxO3a is a transcriptional regulator of ALPP.

### EGFR TKI Sustains Upregulation of ALPP

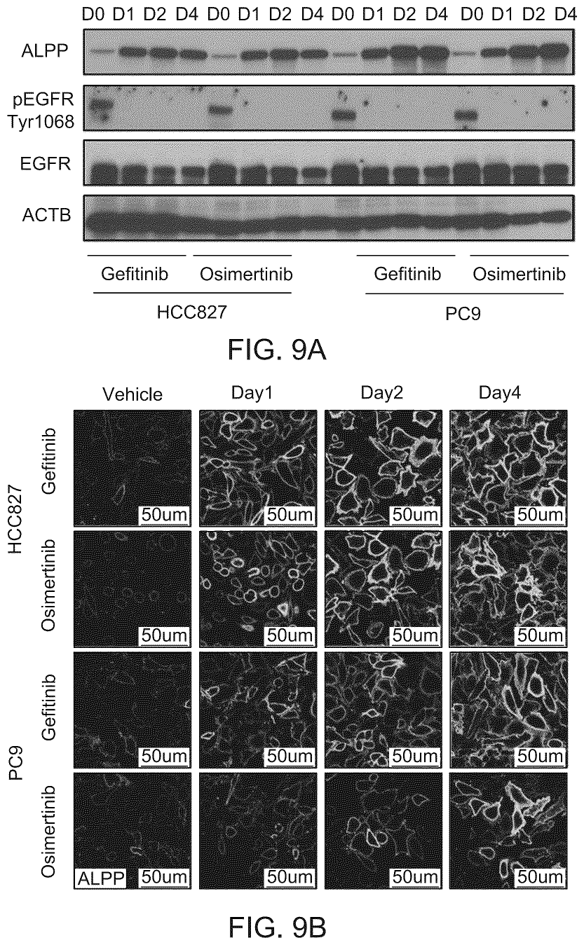

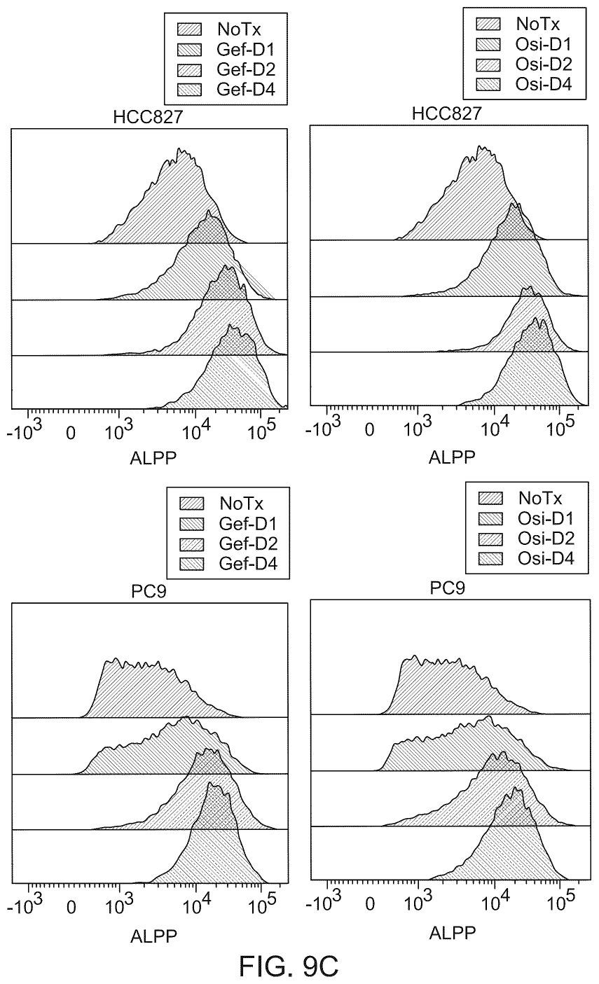

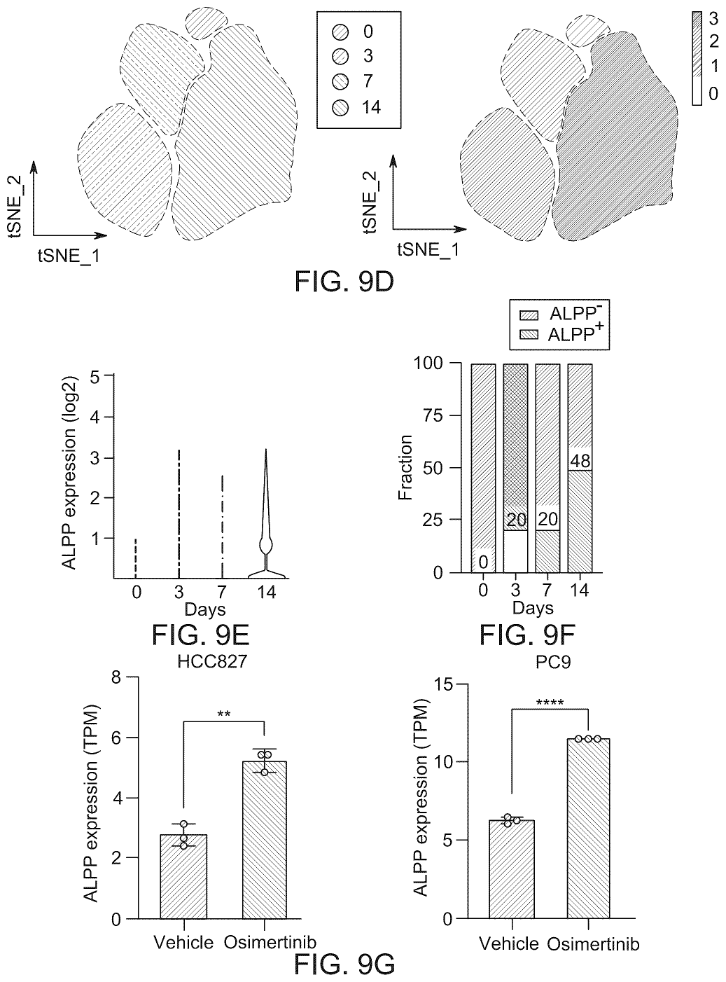

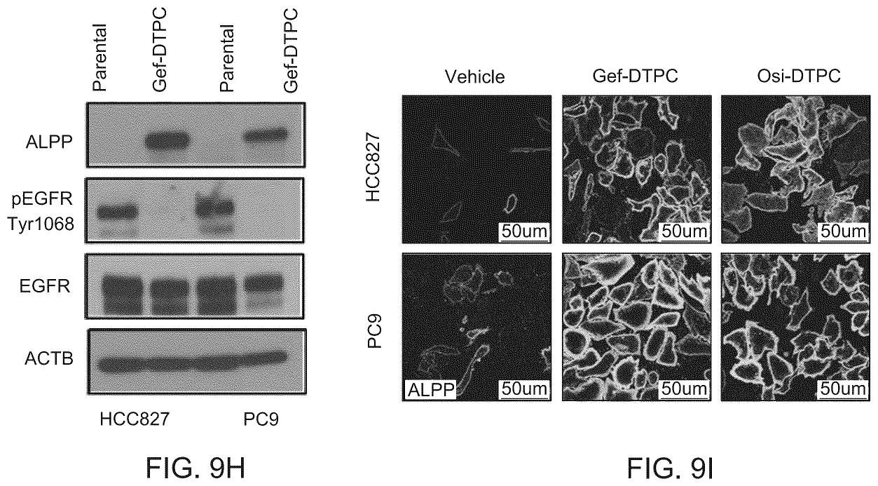

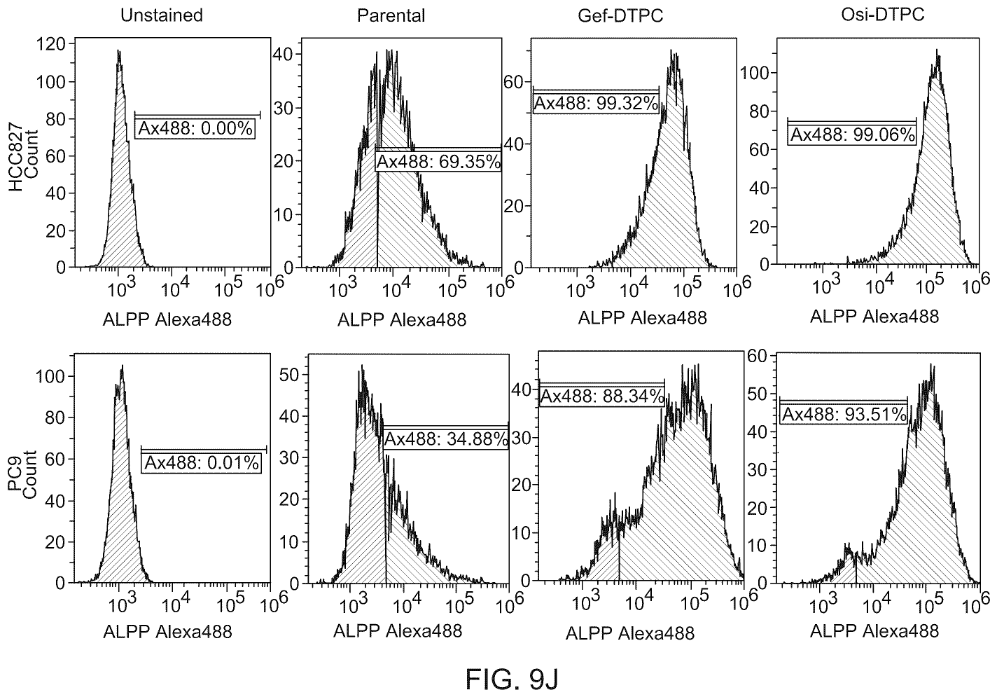

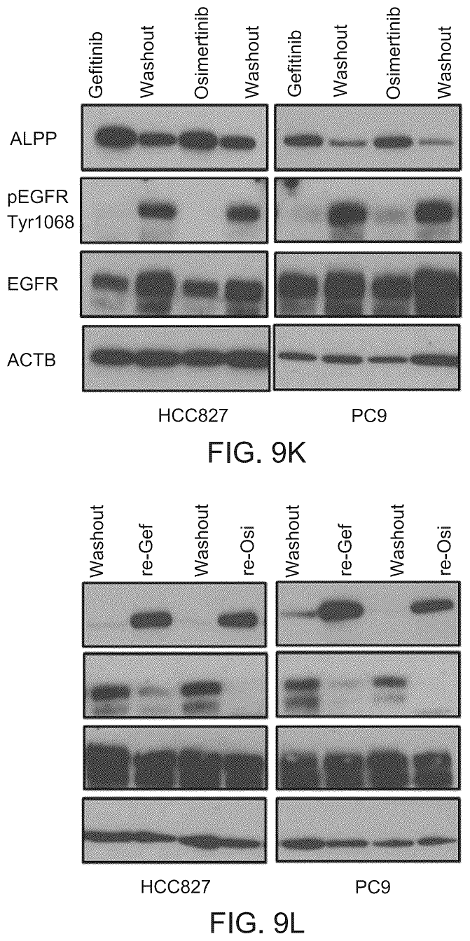

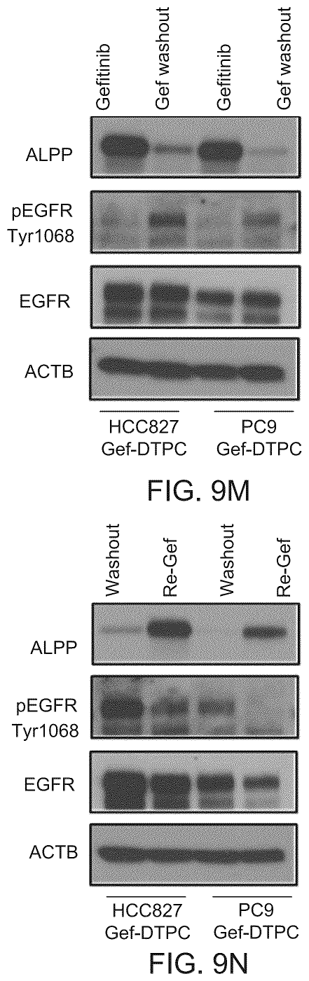

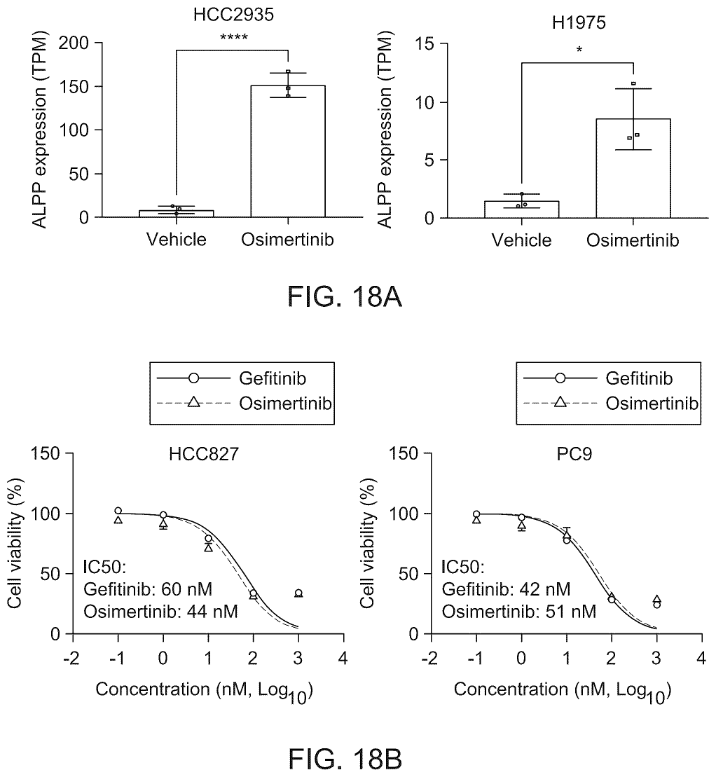

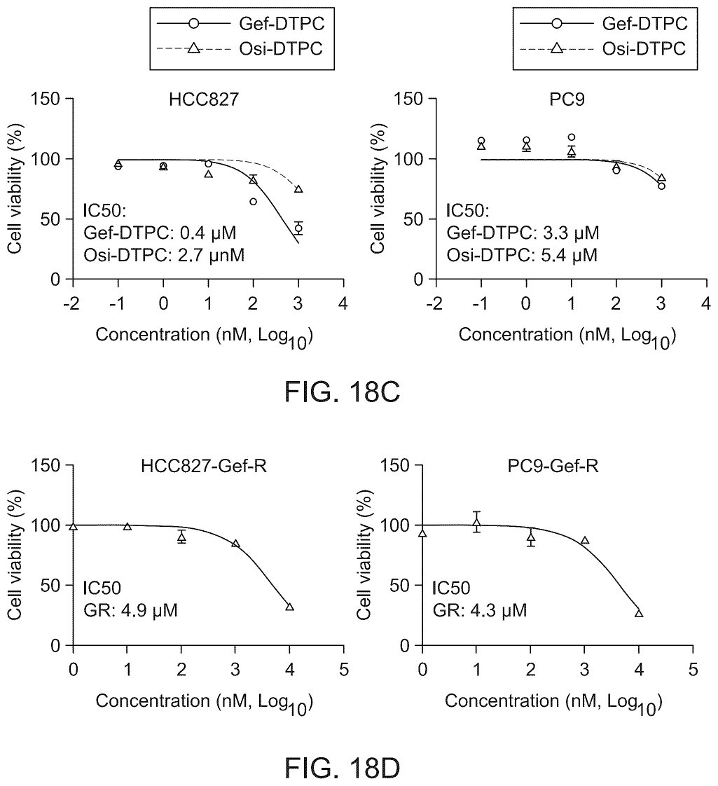

Treatment of HCC827 and PC9 cells with gefitinib or osimertinib for one to four days led to gradual and sustained increase of ALPP and percentage of ALPP⁺ cells (FIG. 9A-FIG. 9C). A prior single cell transcriptomic study involved PC9 cells treated with osimertinib. This dataset was interrogated for expression of ALPP and found a progressive increase in ALPP gene expression following osimertinib treatment (FIG. 9D-FIG. 9E). Interestingly, the ratio of ALPP positive cells was drastically increased following osimertinib treatment, with less than 1% of LUAD cells expressing basal ALPP on day 0 to nearly 50% of LUAD cells expressing ALPP by day 14 (FIG. 9F). Drug tolerant persister cells (DTPCs) are the cell populations that survive and persist drug treatment and contribute to subsequent therapy resistance and disease relapse. It was determined that ALPP expression is maintained in DTPCs and observed sustained expression in osimertinib DTPCs (FIG. 9G and FIG. 18A). DTPCs were further generated from HCC9827 and PC9 LUAD cell lines (FIG. 18B-FIG. 18C) and assessed ALPP expression in Gef-DTPCs and Osi-DTPCs, both of which displayed sustained ALPP expression (FIG. 9H-FIG. 9I), suggesting maintained ALPP expression by EGFR TKIs in drug tolerant cells. The ALPP+Gef-DTPCs and Osi-DTPCs also remarkably increased (FIG. 9J).

To determine if continuous treatment with EGFR inhibitors is required for sustained ALPP expression, HCC827 and PC9 cells were treated with gefitinib or osimertinib for 48 hours and followed for another 48 hours in the absence of inhibitor. ALPP expression was markedly reduced in the absence of EGFR inhibitors and was restored with the addition of inhibitors (FIG. 9K-FIG. 9L). Similar results were observed in Gef-DTPCs (FIG. 9M-FIG. 9N).

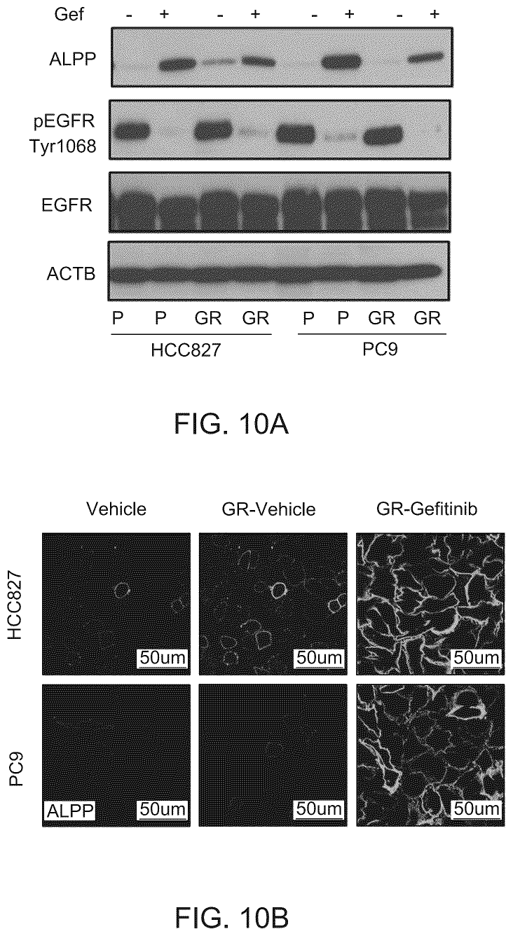

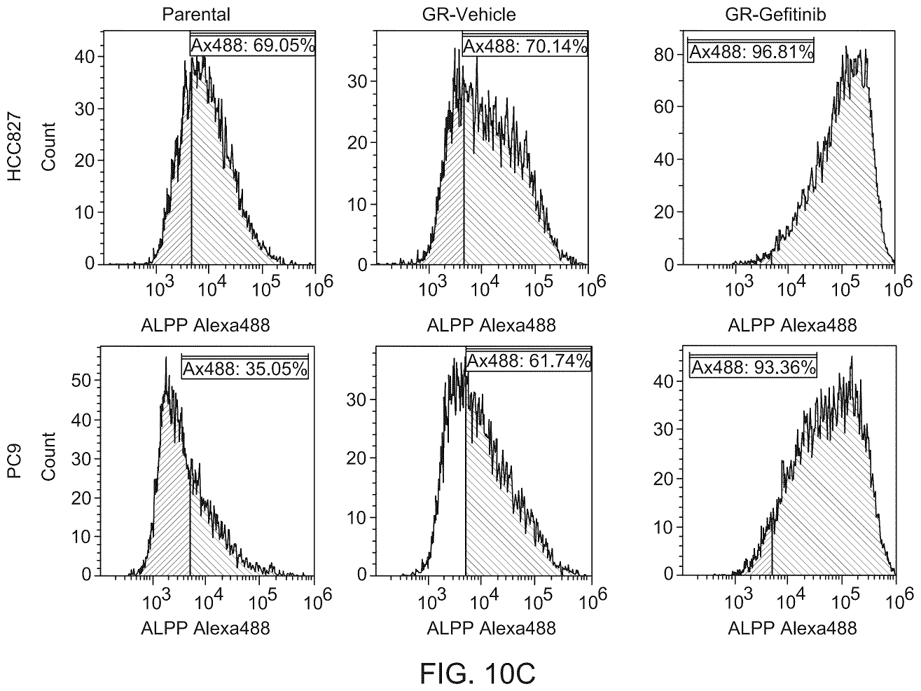

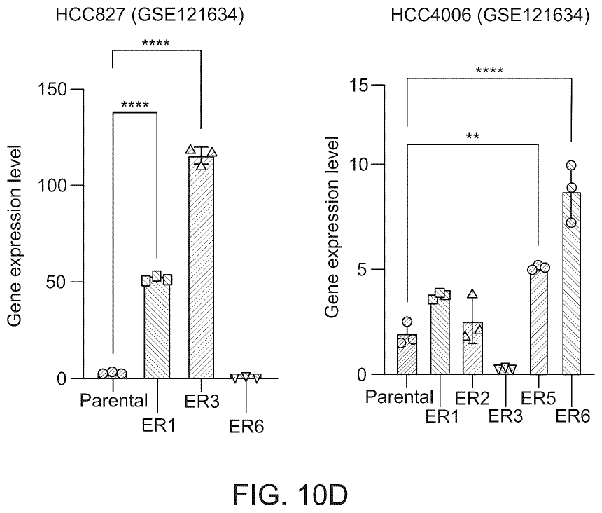

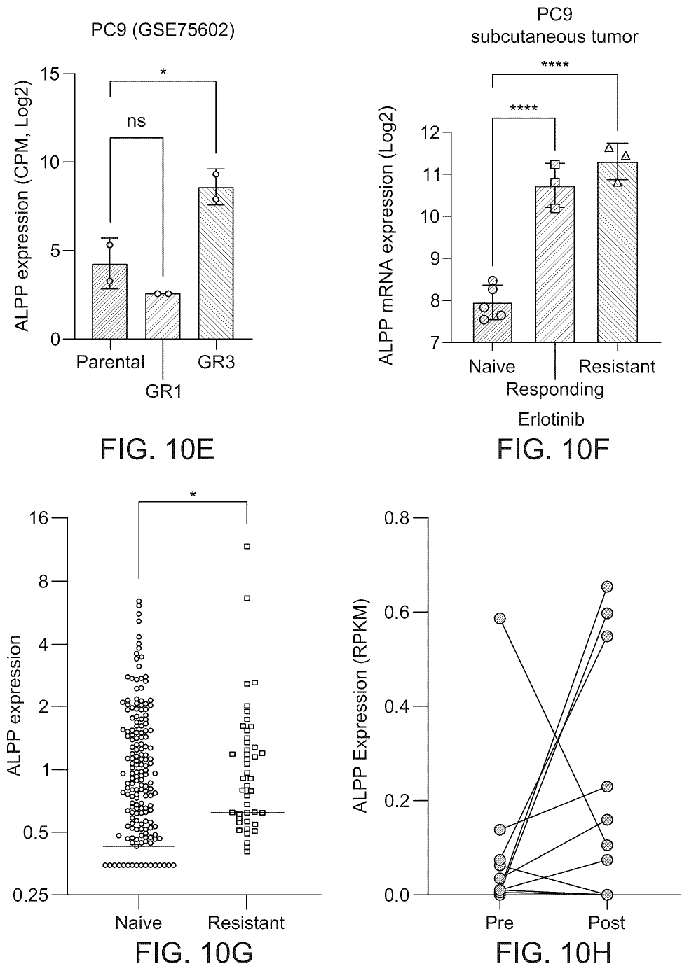

To further explore whether ALPP expression is upregulated in EGFR inhibitor resistant cells, gefitinib-resistant (GR) HCC827 and PC9 cells were generated (FIG. 18D). ALPP expression was higher in GR cells compared with parental cells, and gefitinib treatment further enhanced ALPP upregulation (FIG. 10A-FIG. 10B). Flow cytometry analysis of ALPP demonstrated the majority of gefitinib-resistant cells expressed high level of ALPP upon gefitinib challenge (FIG. 10C). These results are concordant with ALPP upregulation in erlotinib-resistant HCC827 and HCC4006 and gefitinib-resistant PC9 LUAD cells (FIG. 10D-FIG. 10E). Transcriptomic analysis of PC9 xenograft tumors receiving erlotinib treatment also revealed increased ALPP mRNA expression in erlotinib-responding tumors compared with treatment naïve tumors, and elevated ALPP gene expression was sustained in erlotinib-resistant tumors (FIG. 10F). Using publicly available transcriptomic data, the transcription of ALPP in treatment-naïve and EGFR TKI-resistant tumors was also examined. Increased levels of ALPP occurred in NSCLC resistant to first- and second-generation EGFR TKI compared to treatment naïve tumors (FIG. 10G). Likewise, a paired comparison of ALPP expression in tumors before and after development of resistance to osimertinib revealed general upregulation of ALPP (FIG. 10H).

Combination of EGFR TKI and ALPP-ADC treatment enhances cancer cell-killing

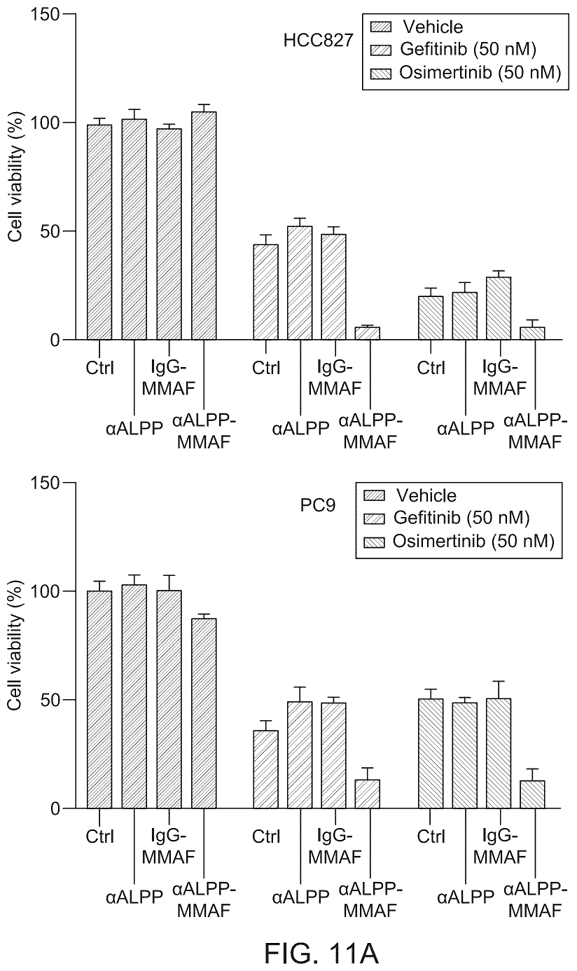

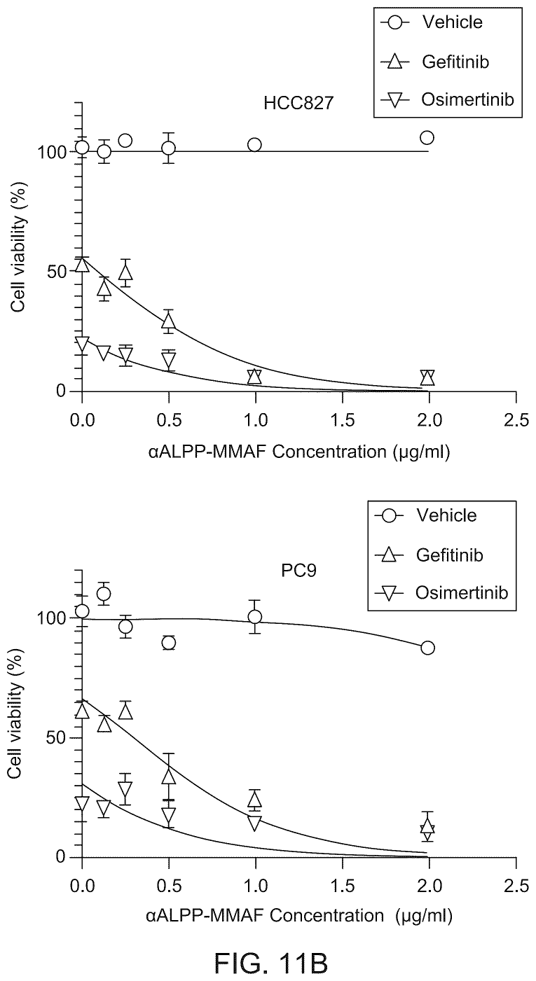

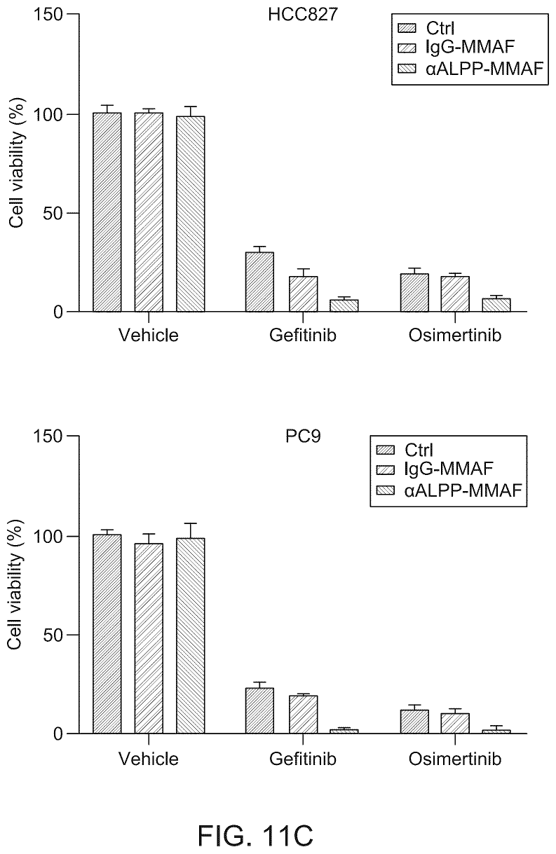

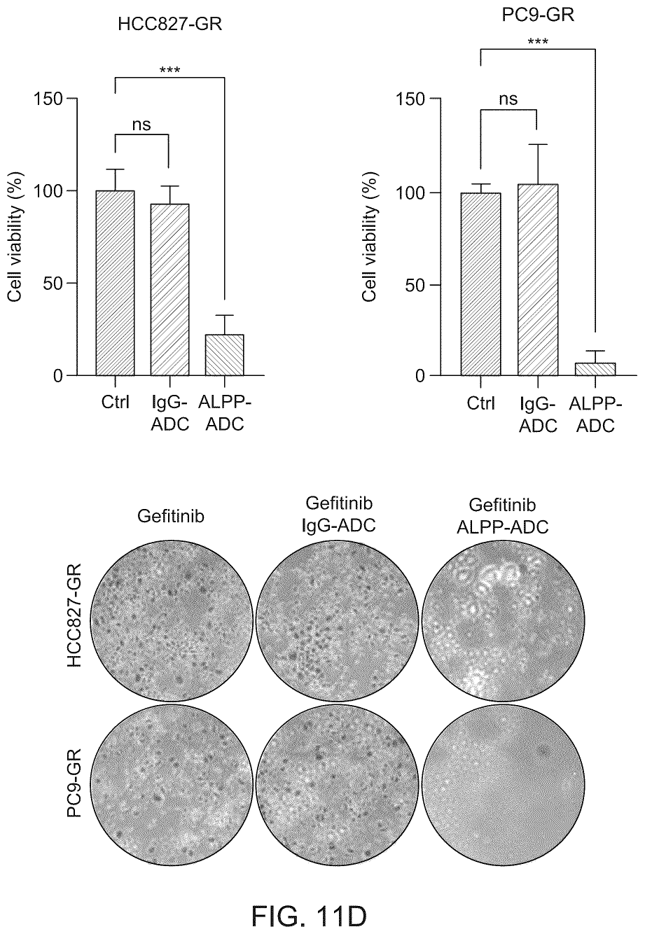

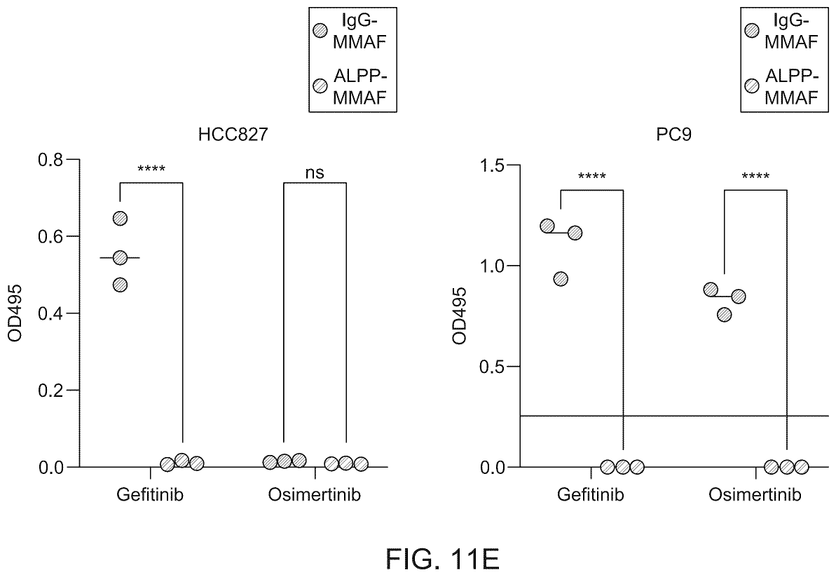

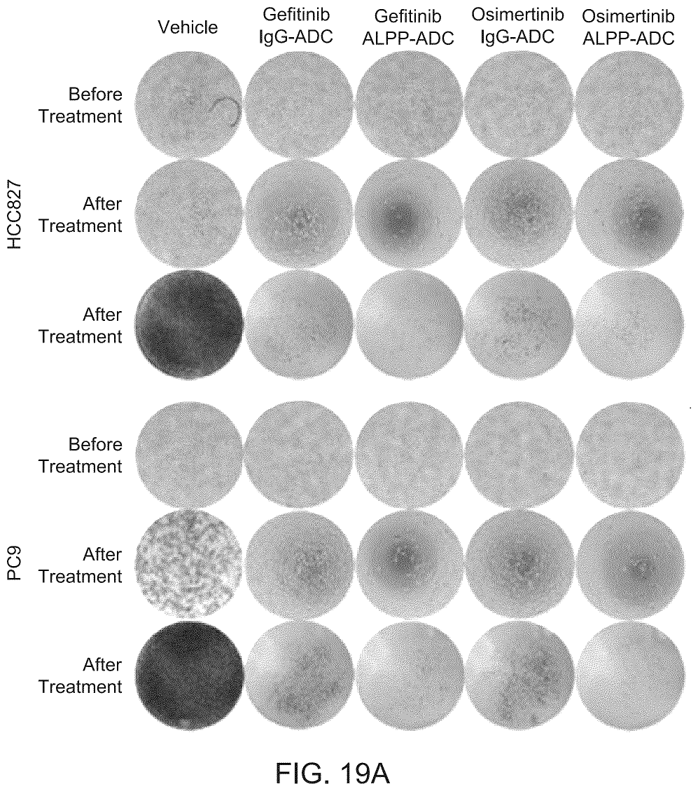

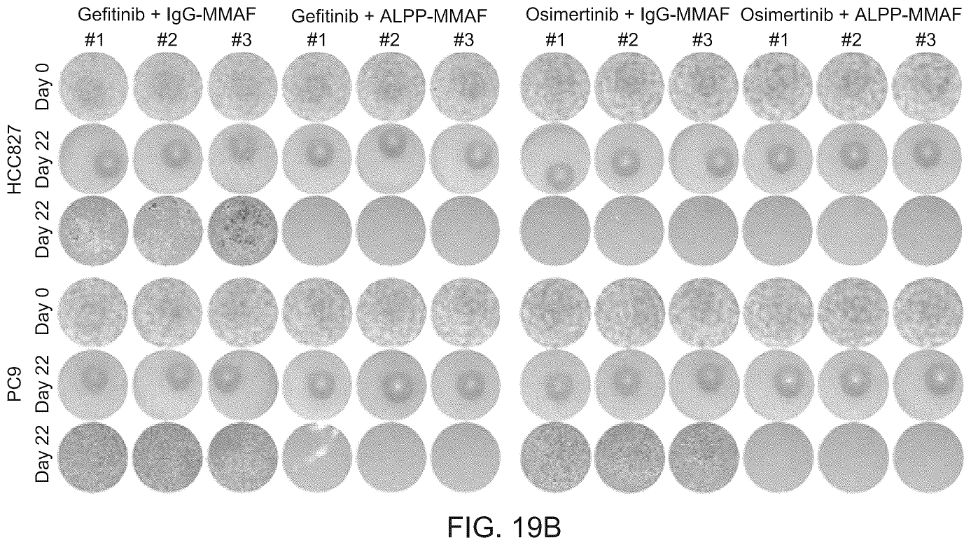

The in vitro efficacy of the combination regimen composing of EGFR TKI and anti-ALPP antibody conjugated with monomethyl auristatin F (MMAF), a microtubule-disrupting agent inducing apoptosis, was evaluated. The combination resulted in increased cell death compared with either treatment alone (FIG. 11A and FIG. 19A). Titration of the ADC revealed dose-dependent cytotoxicity of ALPP-MMAF (FIG. 11B). Treatment with gefitinib or osimertinib followed by ALPP-MMAF treatment also resulted in robust cytotoxicity (FIG. 11C). Likewise, ALPP-MMAF showed effectiveness in gefitinib-resistant HCC827 and PC9 cells (FIG. 11D). Continuous treatment of HCC827 and PC9 cells with gefitinib or osimertinib led to the emergence of drug-resistant clones, while the addition of ALPP-MMAF in gefitinib or osimertinib-treated HCC827 and PC9 cells effectively repressed the formation of EGFR TKI resistant clones (FIG. 11E and FIG. 19B).

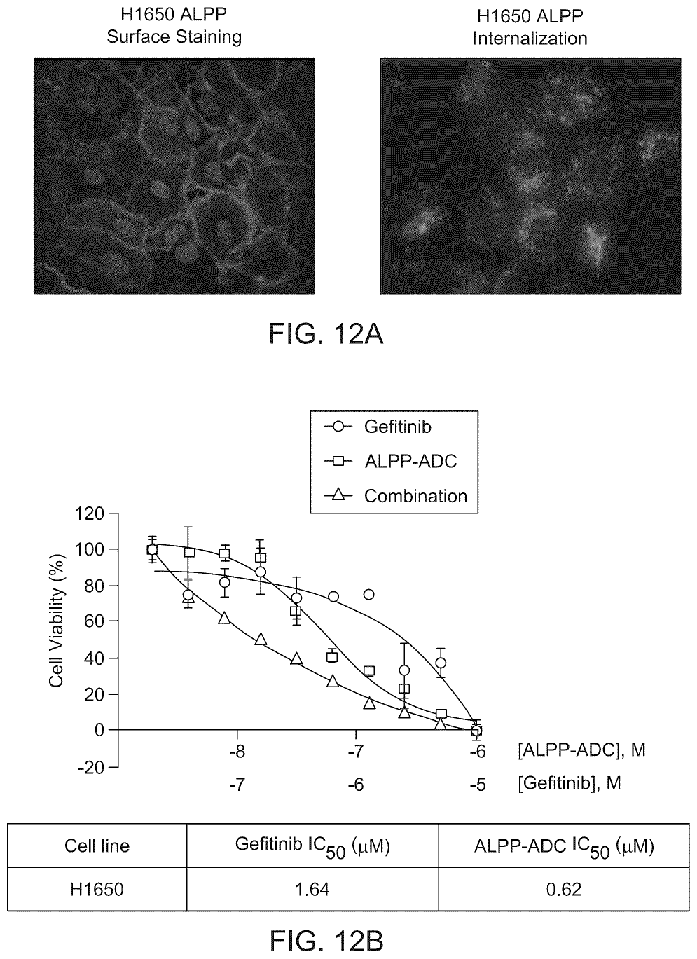

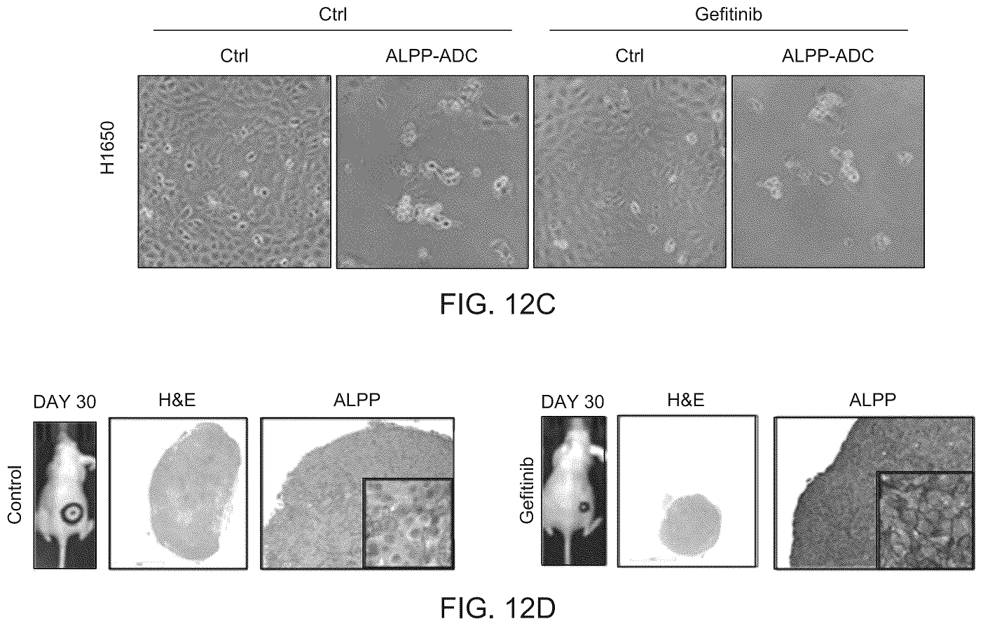

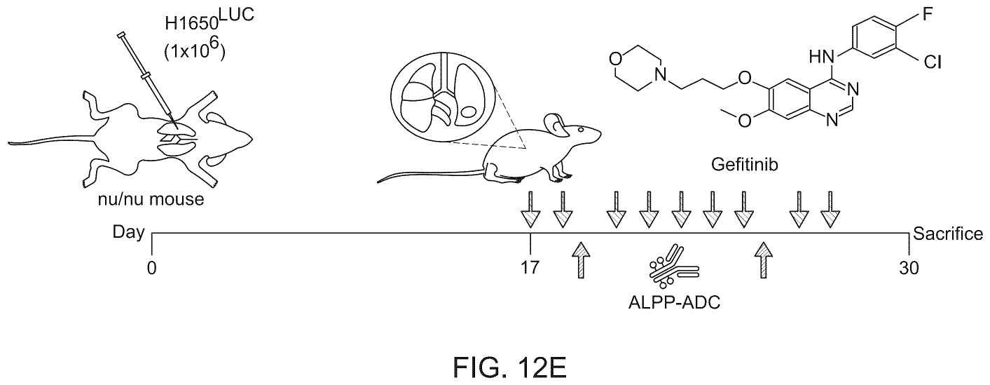

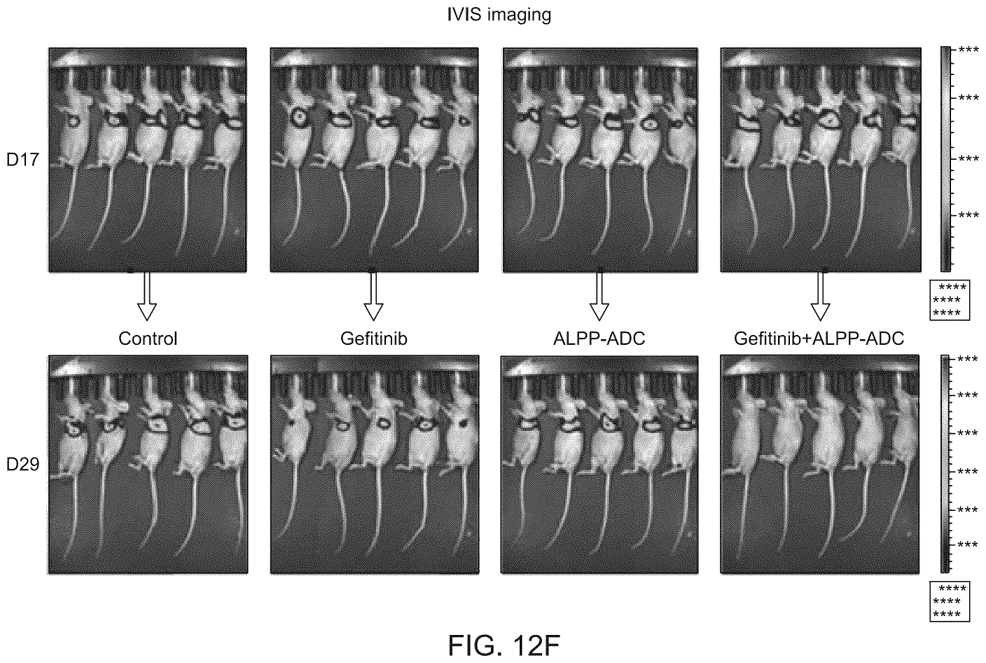

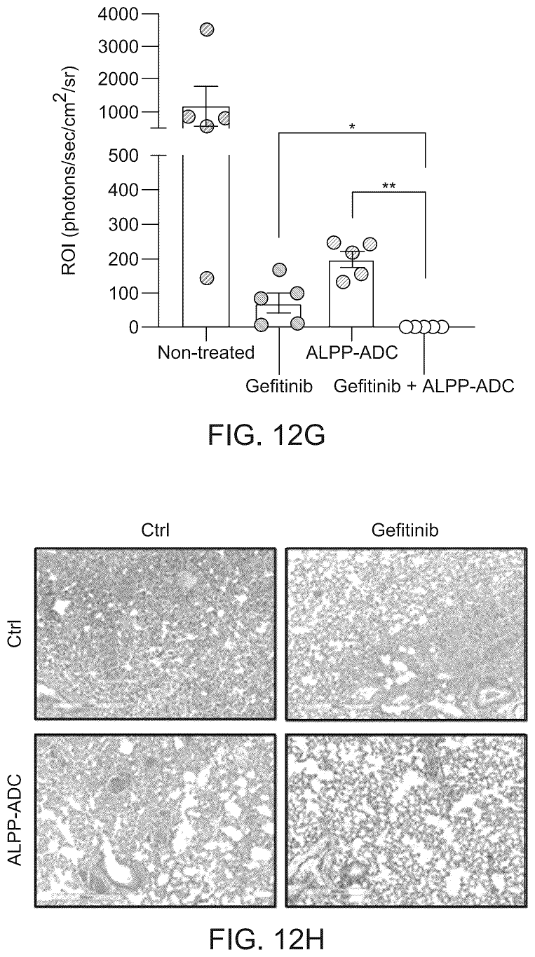

The dual regimen in gefitinib resistant cell line H1650 was then tested. Surface ALPP was targeted using Phrodo-Red conjugated ALPP antibody in H1650 LUAD cells and demonstrated internalization (FIG. 12A). The combination treatment showed synergistic effects with potent cancer-killing effects (FIG. 12B-FIG. 12C).

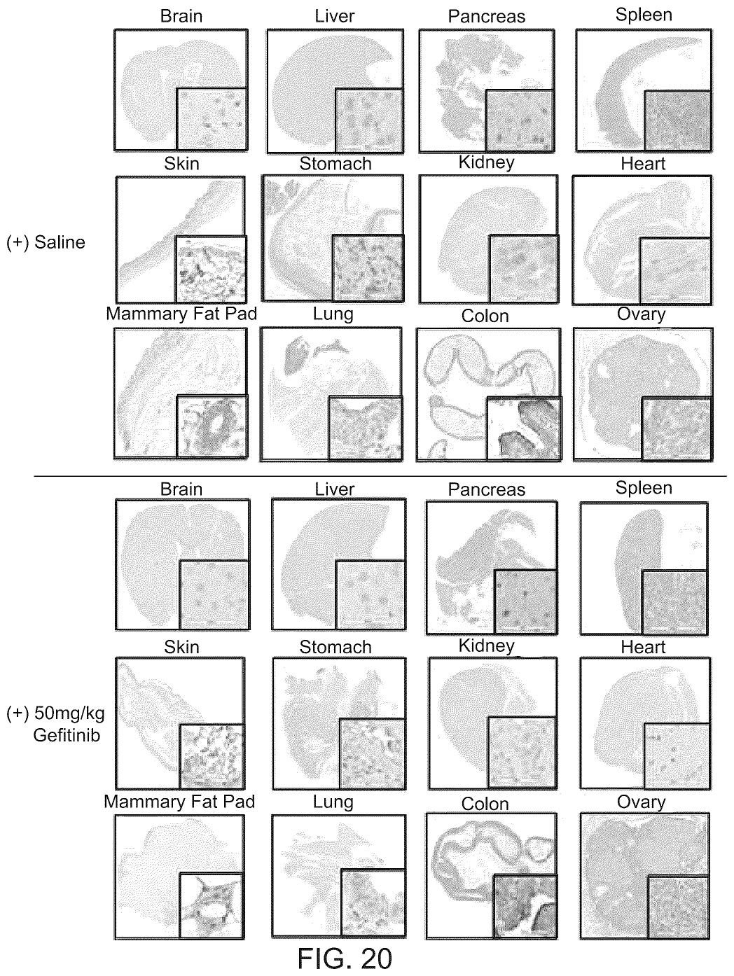

Furthermore, firefly luciferase-expressing H1650 (H1650^{Luc}) LUAD cells were orthotopically implanted into BALB/c nude mice and treated tumor-bearing mice daily with 50 mg/kg gefitinib or saline control for 10 days. Tumor and other organ tissues were subsequently harvested, and ALPP expression assessed via IHC. Consistent with the in vitro observations, tumor-bearing mice treated with gefitinib exhibited pronounced increases in tumoral ALPP surface expression (FIG. 12D). ALPP protein staining in other tissues were negative or showed no appreciable difference compared to the vehicle control (FIG. 20). Using an independent cohort of H1650^{Luc} tumor-bearing mice, the anti-cancer efficacy of 50 mg/kg gefitinib, 5 mg/kg ALPP-MMAF were assessed alone, and in combination (FIG. 12E). Treatment with gefitinib or ALPP-MMAF alone resulted in a statistically significant reduction in relative fluorescence units (RFU) of H1650^{Luc} tumors compared to the control. Remarkably, tumor-bearing mice treated with gefitinib plus ALPP-MMAF exhibited complete responses and were tumor-free based on IVIS imaging (FIG. 12F-FIG. 12G).

### Discussion

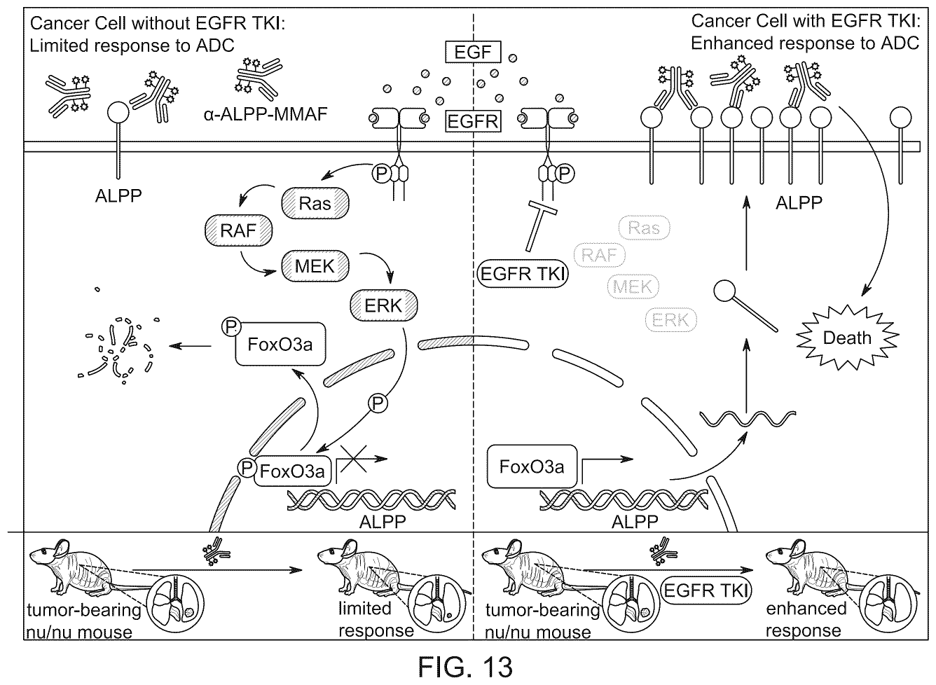

EGFR TKI monotherapy is the first-line treatment for lung cancer patients harboring EGFR activating mutations. Despite the high initial response to these medications, resistance to EGFR TKIs inevitably emerges, resulting in cancer progression within 1-2 years post the onset of therapy. A combination therapy can maximize anti-tumor efficacy and substantially impede the development of drug resistance. Here it was demonstrated that EGFR inhibitor-driven ALPP upregulation provides a feasible ‘two-hit’ combination regimen comprising EGFR TKI and ALPP-MMAF with improved efficacy against TKI-sensitive and -refractory cancer cells (FIG. 13).

ALPP is a GPI-anchored protein that expresses on the cell surface in some cancers, whereas normal tissue expression is largely limited to the placenta and is currently being explored as a target for cancer therapy. However, the expression levels of ALPP in most cancer types are relatively low. In lung cancers, ALPP is predominantly expressed in adenocarcinoma. Consistent with prior studies, assessment of ALPP mRNA expression and surface protein expression in human lung tumors and cancer cell lines demonstrated variable expression with outliers being frequently observed in LUAD. In the TMA of lung tumors, ALPP surface staining was found in 11.3% of all lung tumors analyzed, with the highest frequency of ALPP surface staining being observed in LUAD (17.6%). Notably, ALPP staining positivity was associated with smoking status. Prior studies have reported that serum levels of ALPP are increased up to 10-fold in individuals that smoke cigarettes compared to non-smokers. Moreover, epigenome-wide association study (EWAs) comparing current, former and never smokers from 1,793 participants revealed that methylation of CpG islands in the ALPP loci for smokers was lower than that of non-smokers. More recently, a study identified ALPP as one of the risk biomarkers for smoking-related lung cancer. Since the data suggested that basal expression of ALPP is required for EGFR TKI-mediated upregulation, repression of EGFR may boost ALPP expression more robustly in patients with smoking history.

Prior studies have documented an inverse relationship between ALPP expression and cell proliferative status. Consistent with these observations, marked ALPP was found to be upregulation in cancer cells that were subjected to EGFR inhibitors. Of importance, enhancement of ALPP expression in cancer cells was transient and reversible, suggesting that ALPP expression may be associated with cell quiescence. In support of this, FoxO3a, a key regulator of cell quiescence, was identified as an upstream transcriptional regulator of ALPP. FoxO3a is one of four related FoxO transcription factors that protect cells against a wide range of physiologic stresses, playing central roles in DNA repair, growth arrest, and apoptosis in response to DNA damage and oxidative stress. Inactivation of FoxO3a, which is frequently reported in various malignancies, has been attributed to overactivation of the PI3K-AKT or MEK-ERK signaling pathways, which negatively regulate the transcriptional activity of FoxO3a via its phosphorylation at Thr32, Ser253 and Ser294 that prompts and sustains nuclear exclusion and proteasomal degradation. Previous studies have well documented that EGFR signaling represses FoxO3a activation. Inhibition of EGFR by erlotinib results in the nuclear accumulation and transcriptional activation of FoxO3a. EGF induced FoxO3a phosphorylation and inactivation is interrupted by gefitinib treatment in NSCLC cell lines. In the present study, it was found that EGFR TKI treatment suppresses FoxO3a phosphorylation in LUAD cells, resulting in FoxO3a nuclear translocation that was met with concomitant increases in ALPP mRNA and protein levels. Inhibition of FoxO3a using the small molecule inhibitor AS1842856 reduced ALPP protein expression and prevented EGFR TKI-mediated ALPP upregulation, and the ChIP-qPCR assay demonstrated the direct binding of FoxO3a to the promoter of ALPP. Collectively, the data identified FoxO3a as a transcriptional regulator of ALPP underlying EGFR TKI-driven upregulation of ALPP.

The findings prompted us to evaluate whether EGFR TKI-mediated enhancement surface ALPP expression in cancer cells may increase the efficacy of ALPP-targeting therapies. To this end, it was demonstrated that combination treatment composing of gefitinib/osimertinib and ALPP-MMAF resulted in markedly improved cancer killing effects compared to either treatment modality alone. Treatment of EGFR-mutant LUAD-tumor bearing mice with gefitinib resulted in increased ALPP surface expression specifically in tumors but not in normal tissues. Combinatorial treatment of EGFR-mutant LUAD-tumor bearing mice with gefitinib plus ALPP-MMAF resulted in remarkable anti-cancer effects, surpassing the efficacy of gefitinib or ALPP-MMAF treatment alone. This synergistic combination approach demonstrates the potential of targeting both EGFR signaling and ALPP expression for enhanced therapeutic outcomes in EGFR-mutant lung cancer.

Acquired drug resistance to EGFR TKIs, which typically arise within 2 years after the initiation of EGFR inhibitor treatment, remains a significant clinic challenge. EGFR-dependent resistance often occurs due to the acquisition of additional EGFR mutations (e.g., EGFR T790M, exon 19 deletion, L858R, or C797S), which happens in approximately 50% of cases for early-generation EGFR TKIs or around 10-20% in patients treated with osimertinib, could be potentially addressed by using different generation of EGFR inhibitors or combinational regimens. EGFR-independent resistance, including the expression of other receptor tyrosine kinases, EMT, and small cell lung cancer transformation. There remains a paucity of therapies for the vast majority of EGFR-independent resistance. The present discovery demonstrated that ALPP is upregulated in cancer cells tightly related to EGFR signaling. Importantly, EGFR TKIs sustain ALPP expression even in the drug-refractory cancer cells. In vitro evaluation of the efficacy of ALPP-MMAF in DTPCs and gefitinib-resistant cells revealed effective killing of gefitinib or osimertinib refractory cancer cells. Moreover, ALPP-MMAF, in combination with gefitinib or osimertinib, also prevented the formation of drug resistant clones. A recent study aimed to identify surface therapeutic targets in EMT-related EGFR TKI-resistant NSCLC also listed ALPP as one of the highly upregulated proteins. These findings support for the feasible application of an ALPP-targeting regimen in addressing EGFR-independent resistance.

In conclusion, the present study provides mechanistic insights into ALPP upregulation in cancer cells and identifies FoxO3a as the transcriptional regulator of ALPP. Importantly, the present studies present a novel combinational therapeutic strategy that leverages enhanced ALPP expression in cancer cells, potentially increasing the efficacy of targeted therapy in LUAD with EGFR activating mutations.

TABLE 4
Human cancer cell lines used in this study

| | | ALPP | | ALPPL2 | |
|---|---|---|---|---|---|
| | | TCE | Surface | TCE | Surface |
| CellLine | Cancer Type | TotalPep | TotalPep | TotalPep | TotalPep |
| H1437 | LungAdeno | 0 | 0 | 0 | 0 |
| H1650 | LungAdeno | 35 | 200 | 0 | 0 |
| H3255 | LungAdeno | 0 | 0 | 0 | 0 |
| HCC4019 | LungAdeno | 0 | 0 | 0 | 0 |
| DFC1032 | LungAdeno | 0 | 0 | 0 | 0 |
| HCC827 | LungAdeno | 0 | 0 | 0 | 0 |
| H1573 | LungAdeno | 0 | 0 | 0 | 0 |
| H1395 | LungAdeno | 0 | 0 | 0 | 0 |
| H969 | LungAdeno | 25 | 407 | 0 | 299 |
| H820 | LungAdeno | 0 | 0 | 0 | 0 |
| H2291 | LungAdeno | 10 | 170 | 0 | 0 |
| HCC4017 | LungAdeno | 0 | 0 | 0 | 0 |
| HCC2935 | LungAdeno | 3 | 8 | 0 | 0 |
| H3255 | LungAdeno | 0 | 0 | 0 | 0 |
| H1299 | LungAdeno | 0 | 0 | 0 | 0 |
| H23 | LungAdeno | 0 | 0 | 0 | 0 |
| H838 | LungAdeno | 0 | 0 | 0 | 0 |
| H2030 | LungAdeno | 0 | 0 | 0 | 0 |
| DFC1024 | LungAdeno | 0 | 0 | 0 | 0 |
| H1355 | LungAdeno | 0 | 0 | 0 | 0 |
| H2405 | LungAdeno | 0 | 0 | 0 | 0 |
| H522 | LungAdeno | 0 | 0 | 0 | 0 |
| H1703 | LungAdeno | 0 | 0 | 0 | 0 |
| PC9 | LungAdeno | 29 | 117 | 0 | 0 |
| H1975 | LungAdeno | 0 | 0 | 0 | 0 |
| H2009 | LungAdeno | 0 | 0 | 0 | 0 |
| H1568 | LungAdeno | 40 | 72 | 0 | 0 |
| H650 | LungAdeno | 0 | 0 | 0 | 0 |
| H1944 | LungAdeno | 0 | 0 | 0 | 0 |
| H1373 | LungAdeno | 0 | 0 | 0 | 0 |
| H1693 | LungAdeno | 0 | 0 | 0 | 0 |
| H1993 | LungAdeno | 7 | 5 | 0 | 0 |
| HCC4011 | LungAdeno | 0 | 0 | 0 | 0 |
| HCC2279 | LungAdeno | 0 | 0 | 0 | 0 |
| H2228 | LungAdeno | 0 | 0 | 0 | 0 |
| H1792 | LungAdeno | 0 | 0 | 0 | 0 |
| H2122 | LungAdeno | 0 | 13 | 0 | 0 |
| HCC4006 | LungAdeno | 0 | 0 | 0 | 0 |
| H647 | LungAdeno | 0 | 0 | 0 | 0 |
| H1385 | LungAdeno | 0 | 0 | 0 | 0 |
| H920 | LungAdeno | 0 | 0 | 0 | 0 |
| H1793 | LungAdeno | 0 | 0 | 0 | 0 |
| H1651 | LungAdeno | 774 | 7557 | 770 | 9063 |
| H1435 | LungAdeno | 0 | 1 | 0 | 0 |
| H2342 | LungAdeno | 3 | 97 | 0 | 0 |
| H1563 | LungAdeno | 170 | 1792 | 55 | 1270 |
| H1838 | LungAdeno | 7 | 0 | 0 | 0 |
| HCC1833 | LungAdeno | 0 | 5 | 0 | 0 |
| H1755 | LungAdeno | 0 | 0 | 0 | 0 |
| H69AR | SCLC | 0 | 0 | 0 | 0 |
| H69 | SCLC | 0 | 0 | 0 | 0 |
| H69CPR | SCLC | 0 | 0 | 0 | 0 |
| H209 | SCLC | 6 | 0 | 0 | 0 |
| H345 | SCLC | 0 | 0 | 0 | 0 |
| H69(P) | SCLC | 0 | 0 | 0 | 0 |
| H69AD | SCLC | 0 | 0 | 0 | 0 |
| HCC4001 | SCLC | 0 | 0 | 0 | 0 |
| HCC4003 | SCLC | 0 | 0 | 0 | 0 |
| HCC4005 | SCLC | 0 | 0 | 0 | 0 |
| H82 | SCLC | 0 | 0 | 0 | 0 |
| H1607 | SCLC | 0 | 71 | 0 | 39 |
| H211 | SCLC | 0 | 0 | 0 | 0 |
| H2195 | SCLC | 0 | 0 | 0 | 0 |
| H82 | SCLC | 0 | 0 | 0 | 0 |
| DMS79 | SCLC | 0 | 0 | 0 | 0 |
| HCC4002 | SCLC | 0 | 0 | 0 | 0 |
| H2679 | SCLC | 0 | 0 | 0 | 0 |
| H524 | SCLC | 0 | 0 | 0 | 0 |
| H526 | SCLC | 0 | 0 | 0 | 0 |
| HCC4004 | SCLC | 0 | 0 | 0 | 0 |
| SCLC | SCLC | 0 | 0 | 0 | 0 |

TABLE 5
Reagents used in this study

| | | |
|---|---|---|
| Reagent or resource | Identifier | Source |

Antibody

| | | |
|---|---|---|
| ALPP | #NB110-3638 | Novus Biologicals |
| ALPP | #ab133602 | Abcam |
| β-actin | #3700 | Cell Signaling |
| | | Technology |
| Phospho-AKT | #9271 | Cell Signaling |
| (Ser473) | | Technology |
| Phospho-ERK | #9101 | Cell Signaling |
| (Thr202/Tyr204) | | Technology |
| Phospho-STAT1 | #9167 | Cell Signaling |
| (Tyr701) | | Technology |
| Phospho-STAT1 | #8826 | Cell Signaling |
| (Ser727) | | Technology |
| AKT | #4691 | Cell Signaling |
| | | Technology |
| ERK | #4695 | Cell Signaling |
| | | Technology |
| STAT1 | #14994 | Cell Signaling |
| | | Technology |
| GAPDH | #5174 | Cell Signaling |
| | | Technology |
| FoxO3a | #720128 | Invitrogen |
| FoxO3a | #12829 | Cell Signaling |
| | | Technology |
| Phospho-FoxO3a | #5538 | Cell Signaling |
| (Ser294) | | Technology |
| Phospho-FoxO3a | #64616 | Cell Signaling |
| (Ser425) | | Technology |
| αMFc-NC-MMAF | #AM-101AF | Moradec LLC |

Biological Samples

| | | |
|---|---|---|
| Human Multi-tissue | #NBP2-78113 | Novus Biologicals |
| Tissue Microarray | | |
| (Normal) | | |
| TMA | This manuscript | The University of |
| | | Texas MD Anderson |
| | | Cancer Center |

Chemicals

| | | |
|---|---|---|
| FoxO inhibitor | AS1842856 | MedChemExpress |
| CellTiter 96 | G3581 | Promega |
| Aqueous One | | |
| Solution Cell | | |
| Proliferation | | |
| Assay | | |
| D-Luciferin | #115144-35-9 | Regis Technologies |
| potassium salt | | Inc |
| Gefitinib | #13166 | Cayman Chemical |
| | | Company |
| Lapatinib | #11493-5 | Cayman Chemical |
| | | Company |
| Afatibib | #S1011 | Selleck Chemicals |
| Osimertinib | #S7297 | Selleck Chemicals |
| EGFR inhibitor | #15363 | Cayman Chemical |
| | | Company |
| EGFR/ERBB2 | #17568 | Cayman Chemical |
| inhibitor | | Company |
| SsoAdvanced | #1725271 | BIO-RAD |
| Universal | | |
| SYBR Green | | |
| Supermix | | |
| 16% Formaldehyde, | #28906 | Thermo Fisher |
| Methanol-free | | Scientific |
| DMSO | #D2650 | Sigma-Aldrich |
| TaqMan Universal | #4440040 | Applied Biosystems |
| Master Mix II, | | |
| no UNG | | |
| NVP-BKM120 | #11587 | Cayman Chemical |
| | | Company |
| AZD8330 | #HY-12058 | MedChemExpress |
| SCH772984 | #HY-50846 | MedChemExpress |

Critical commerical assays

| | | |
|---|---|---|
| H&E staining kit | #Ab245880 | Abcam |
| Cell Fractionation | #9038 | Cell Signaling |
| kit | | Technology |
| Pierce Magnetic | #26157 | Thermo Fisher |
| ChIP kit | | Scientific |

Experimental models: Cell lines

| | | |
|---|---|---|
| cancer cell lines | ATCC | Table 4 |
| H1650-Lucferase | This manuscript | N/A |
| HCC827-Lucferase | This manuscript | N/A |
| H1650-RFP | This manuscript | N/A |
| H1650-FoxO3a | This manuscript | N/A |
| HCC827-RFP | This manuscript | N/A |
| HCC827-FoxO3a | This manuscript | N/A |

Experimental models: Organisms/strains

| | | |
|---|---|---|
| pLOC-RFP lentivirus | OHS5832 | MD Anderson |
| | | Functional |
| | | Genomics Core |
| pLOC-hFoxO3a | PLOHS_ccsbBEn_{—} | MD Anderson |
| lentivirus | 00577 | Functional |
| | | Genomics Core |
| Firefly Luciferase | #LPP-FLUC- | GeneCopoeia |
| Lentifect Purified | Lv105-100 | |
| Lentiviral Particles | | |
| Mouse: BLAB/c nude | #194 | Charles River |
| mice | | |

Oligonucleotides

| | | |
|---|---|---|
| ALPP taqman probe | Hs03046558_s1 | Thermo Fisher |
| | | Scientific |
| RT-qPCR primers for | This manuscript | Thermo Fisher |
| ALPP promoter | | Scientific |

Software and algorithms

GraphPad
IVIS

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating a cancer co-expressing alkaline phosphatase (ALPP) and/or ALPP2 proteins and harboring an EGFR activating mutation in a patient in need thereof, comprising administering to the patient an antibody-drug conjugate targeting ALPP and/or ALPP2 in a cell in combination with an EGFR inhibitor.

2. A method for treating a cancer co-expressing alkaline phosphatase (ALPP) and/or ALPP2 proteins and harboring an EGFR activating mutation in a patient in need thereof comprising:
identifying the EGFR activating mutation in a cell from a biological sample obtained from the patient;
detecting and/or quantifying ALPP and/or ALPP2 cell surface expression in the cell; and
administering an antibody-drug conjugate therapy targeting ALPP and/or ALPP2 in the cancer cell in combination with an EGFR inhibitor.

3. (canceled)

4. A method of preventing emergence of resistance of a cancer cell co-expressing alkaline phosphatase (ALPP) and/or ALPP2 proteins to an EGFR inhibitor comprising administering an antibody-drug conjugate therapy targeting ALPP and/or ALPP2 in the cancer cell in combination with the EGFR inhibitor.

5. The method of claim 1, wherein the cancer cells comprise increased cell surface expression of ALPP and/or ALPP2 relative to healthy cells.

6. The method of claim 1, wherein the EGFR mutation comprises an exon 19 deletion, a T790M point mutation, and/or an L858R point mutation.

7. The method of claim 1, wherein the cancer having an EGFR mutation is resistant to an inhibitor targeting EGFR.

8. The method of claim 1, wherein the cancer cell is a drug-tolerant persister cell (DTPC) or a drug-resistant cell (DRC).

9. The method of claim 1, wherein the administration of the antibody-drug conjugate therapy targeting ALPP and/or ALPP2 in the cancer cell, and the EGFR inhibitor prevents the drug-tolerant persister cell (DTPC) from developing into a drug-resistant cell (DRC).

10. The method of claim 1, wherein the method treats drug-tolerant persister cells (DTPCs) and drug-resistant cells (DRCs) to prevent the emergence of resistance to EGFR inhibitors.

11. The method of claim 1, wherein the cancer co-expressing alkaline phosphatase (ALPP) and/or ALPP2 proteins is ovarian cancer, breast cancer, cervical cancer, endometrial cancer, pancreatic cancer, gastric cancer, colorectal cancer, lung cancer, urothelial cancer, brain cancer, testicular cancer, seminoma, and mesothelioma.

12. The method of claim 1, wherein the cancer is testicular germ cell tumors, uterine corpus endometrial carcinoma, pancreatic adenocarcinoma, pancreatic ductal adenocarcinoma, bladder urothelial carcinoma, triple-negative breast cancer, stomach adenocarcinoma, esophageal carcinoma, uterine carcinosarcoma, rectum adenocarcinoma, head and neck squamous cell carcinoma, lung adenocarcinoma, lung squamous cell carcinoma, small cell lung cancer, non-small cell lung cancer, clone adenocarcinoma, mesothelioma, and acute myeloid leukemia.

13. The method of claim 11, wherein the lung cancer is non-small cell lung cancer.

14. The method of claim 1, wherein detecting and/or quantifying placental alkaline phosphatase (ALPP) and/or ALPP2 cell surface expression in the cancer cell comprises histological analysis, immunohistochemical (IHC) staining for ALPP protein, a blood-based test, a tissue-based test, or imaging techniques.

15. The method of claim 14, wherein the tissue-based test comprises a tissue biopsy, flow cytometry, immunohistochemistry (IHC), western blot (WB), polymerase chain reaction (PCR), or immunofluorescence (IF).

16. (canceled)

17. The method of claim 14, wherein the blood-based test comprises Galleri®, circulating tumor cell (CTC) test, a complete blood count (CBC), or a test or assay for measuring circulating proteins, autoantibodies, cell-free circulating DNA, or extracellular vesicle-derived proteins.

18. The method of claim 15, wherein the tissue biopsy is analyzed by hematoxylin and eosin (H&E) staining and/or microscopy.

19. (canceled)

20. The method of claim 1, wherein the EGFR inhibitor comprises a tyrosine kinase inhibitor (TKI).

21. (canceled)

22. The method of claim 1, wherein the EGFR inhibitor is selected from gefitinib, osimertinib, mobocertinib, amivantamab, CLN081, and/or DZD9008.

23. The method of claim 19, wherein the antibody-drug conjugate comprises SGN-ALPV, Adcetris®, Kadcyla®, Besponsa®, Mylotarg®, Polivy®, Padcev®, Enhertu®, Trodelvy®, Blenrep®, Zynlonta™, Akalux®, Aidixi®, and Tivdak®.

24-25. (canceled)

26. A method for treating cancer harboring an EGFR activating mutation in a patient in need thereof comprising: A method for treating cancer harboring an EGFR activating mutation in a patient in need thereof comprising:
administering an antibody-drug conjugate targeting ALPP and/or ALPP2 on the surface of the cancer cells;
wherein the cancer cells exhibit increased expression of ALPP and/or ALPP2 relative to a healthy cell;
wherein the antibody-drug conjugate targeting ALPP and/or ALPP2 comprises an antibody targeting ALPP and/or ALPP2 conjugated to MMAF;
wherein the cancer cells comprise an activating mutation in the EGFR gene resulting in resistance to EGFR tyrosine kinase inhibitors.
